SEQUENCE LISTING (420 sequences)

SEQ ID NO: 1 - DNA, 3220 bp, Homo sapiens
  source 1..3220: mol_type = genomic DNA
acaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 2 - DNA, 3199 bp, Homo sapiens
  source 1..3199: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 3 - DNA, 3513 bp, Homo sapiens
  source 1..3513: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcgcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 4 - DNA, 3236 bp, Homo sapiens
  source 1..3236: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 5 - DNA, 3532 bp, Homo sapiens
  source 1..3532: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 6 - DNA, 3340 bp, Homo sapiens
  source 1..3340: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 7 - DNA, 3495 bp, Homo sapiens
  source 1..3495: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 8 - DNA, 3492 bp, Homo sapiens
  source 1..3492: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 9 - DNA, 3510 bp, Homo sapiens
  source 1..3510: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcgcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 10 - DNA, 3303 bp, Homo sapiens
  source 1..3303: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 11 - DNA, 3300 bp, Homo sapiens
  source 1..3300: mol_type = genomic DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 12 - DNA, 1321 bp, Macaca mulatta
  source 1..1321: mol_type = genomic DNA
caggacaatgccatcttctgtctcatggggcgtcctcctgctggcaggcctgtgctgcctgctccccggctctctggctgaggatccccagggagatgctgcccagaagacggatacatcccaccatgatcaggaccacccaaccctcaacaagatcacccccagcctggctgagttcggcttcagcctataccgccagctggcacaccagtccaacagcaccaatatcttcttctccccagtgagcatcgctacagcctttgcaatgctctccctggggaccaaggctgacactcacagtgaaatcctggagggcctgaatttcaacgtcacggagattccggaggctcaggtccatgaaggcttccaggaactcctccataccctcaacaagccagacagccagctccagctgaccaccggcaacggcctgttcctcaacaagagcctgaaggtagtggataagtttttggaggatgtcaaaaaactgtaccactcagaagccttctctgtcaactttgaggacaccgaagaggccaagaaacagatcaacaattacgtggagaaggaaactcaagggaaaattgtggatttggtcaaggagcttgacagagacacagtttttgctctggtgaattacatcttctttaaaggcaaatgggagagaccctttgacgttgaggccaccaaggaagaggacttccacgtggaccaggcgaccaccgtgaaggtgcccatgatgaggcgtttaggcatgtttaacatctaccactgtgagaagctgtccagctgggtgctgctgatgaaatacctgggcaatgccaccgccatcttcttcctgcctgatgaggggaaactgcagcacctggaaaatgaactcacccatgatatcatcaccaagttcctggaaaatgaaaacagcaggtctgccaacttacatttacccagactggccattactggaacctatgatctgaagacagtcctgggccacctgggtatcactaaggtcttcagcaatggggctgacctctcggggatcacggaggaggcacccctgaagctctccaaggccgtgcataaggctgtgctgaccatcgatgagaaagggactgaagctgctggggccatgtttttagaggccatacccatgtctattccccccgaggtcaagttcaacaaaccctttgtcttcttaatgattgaacaaaataccaagtctcccctcttcatgggaaaagtggtgaatcccacccagaaataactgcctgtcactcctcagcccctcccctccatccctggccccctccctgaatgacat

SEQ ID NO: 13 - DNA, 2064 bp, Macaca mulatta
  source 1..2064: mol_type = genomic DNA
gcccagtcttgtgtctgcctggcaatgggcaaggccccttcctgcccaagctccccgcccctccccaacctattgcctccgccacccgccacccgaggccaacttcctgggtgggcaggaactgggccctgtgcccagggcgtgcactgcctccacgcagcaaccctcagagtactgagctgagcaaaggaggaggaggggatcagcactctgaggaaggcctagccactgctgctgccaggaattccagggcggcatcagtcttcagcatcaggcatttcggggtgaattagtaaatggtagatcttgctaccagtggaacagccgctaaggattctgcagtgagagcagagggccagcaaagtggtactctcccagcgactggctgactcacgccaccccctccaccttggacgcaggacactgtggtttctgagccaggtacaatgactccttttggtacgtgcagtggaggctgtatgctgctcaggcagagcgtccggacagcgtgggcgggcgactcagcgcccagcctgtgaacttagtccctgtttgctcctccggtaactggggtgatcttggttaatattcaccagcagcctcccccgttgcccctctgcacccactgcttaaatacggacaaggacagggctctgtctcctcagcctcaggcaccaccactgacctgggacggtgaatcgacaatgccatcttctgtctcatggggcgtcctcctgctggcaggcctgtgctgcctgctccccggctctctggctgaggatccccagggagatgctgcccagaagacggatacatcccaccatgatcaggaccacccaaccctcaacaagatcacccccagcctggctgagttcggcttcagcctataccgccagctggcacaccagtccaacagcaccaatatcttcttctccccagtgagcatcgctacagcctttgcaatgctctccctggggaccaaggctgacactcacagtgaaatcctggagggcctgaatttcaacgtcacggagattccggaggctcaggtccatgaaggcttccaggaactcctccataccctcaacaagccagacagccagctccagctgaccaccggcaacggcctgttcctcaacaagagcctgaaggtagtggataagtttttggaggatgtcaaaaaactgtaccactcagaagccttctctgtcaactttgaggacaccgaagaggccaagaaacagatcaacaattacgtggagaaggaaactcaagggaaaattgtggatttggtcaaggagcttgacagagacacagtttttgctctggtgaattacatcttctttaaaggcaaatgggagagaccctttgacgttgaggccaccaaggaagaggacttccacgtggaccaggcgaccaccgtgaaggtgcccatgatgaggcgtttaggcatgtttaacatctaccactgtgagaagctgtccagctgggtgctgctgatgaaatacctgggcaatgccaccgccatcttcttcctgcctgatgaggggaaactgcagcacctggaaaatgaactcacccatgatatcatcaccaagttcctggaaaatgaaaacagcaggtctgccaacttacatttacccagactggccattactggaacctatgatctgaagacagtcctgggccacctgggtatcactaaggtcttcagcaatggggctgacctctcggggatcacggaggaggcacccctgaagctctccaaggccgtgcataaggctgtgctgaccatcgatgagaaagggactgaagctgctggggccatgtttttagaggccatacccatgtctattccccccgaggtcaagttcaacaaaccctttgtcttcttaatgattgaacaaaataccaagtctcccctcttcatgggaaaagtggtgaatcccacccagaaataactgcctgtcactcctcagcccctcccctccatccctggccccctccctgaatgacattaaagaagggttgagctggtccctgcctgcgtgtgtgactgcaaac

SEQ ID NO: 14 - DNA, 1608 bp, Macaca mulatta
  source 1..1608: mol_type = genomic DNA
tcttgtgtctgcctggcaatgggcaaggccccttcctgcccaagctccccgcccctccccaacctattgcctccgccacccgccacccgaggccaacttcctgggtgggcaggaactgggccctgtgcccagggcgtgcactgcctccacgcagcaaccctcagagtactgagctgagcaaaggaggaggaggggatcagcactctgaggaaggcctagccactgctgctgccaggaattccaggacaatgccatcttctgtctcatggggcgtcctcctgctggcaggcctgtgctgcctgctccccggctctctggctgaggatccccagggagatgctgcccagaagacggatacatcccaccatgatcaggaccacccaaccctcaacaagatcacccccagcctggctgagttcggcttcagcctataccgccagctggcacaccagtccaacagcaccaatatcttcttctccccagtgagcatcgctacagcctttgcaatgctctccctggggaccaaggctgacactcacagtgaaatcctggagggcctgaatttcaacgtcacggagattccggaggctcaggtccatgaaggcttccaggaactcctccataccctcaacaagccagacagccagctccagctgaccaccggcaacggcctgttcctcaacaagagcctgaaggtagtggataagtttttggaggatgtcaaaaaactgtaccactcagaagccttctctgtcaactttgaggacaccgaagaggccaagaaacagatcaacaattacgtggagaaggaaactcaagggaaaattgtggatttggtcaaggagcttgacagagacacagtttttgctctggtgaattacatcttctttaaaggcaaatgggagagaccctttgacgttgaggccaccaaggaagaggacttccacgtggaccaggcgaccaccgtgaaggtgcccatgatgaggcgtttaggcatgtttaacatctaccactgtgagaagctgtccagctgggtgctgctgatgaaatacctgggcaatgccaccgccatcttcttcctgcctgatgaggggaaactgcagcacctggaaaatgaactcacccatgatatcatcaccaagttcctggaaaatgaaaacagcaggtctgccaacttacatttacccagactggccattactggaacctatgatctgaagacagtcctgggccacctgggtatcactaaggtcttcagcaatggggctgacctctcggggatcacggaggaggcacccctgaagctctccaaggccgtgcataaggctgtgctgaccatcgatgagaaagggactgaagctgctggggccatgtttttagaggccatacccatgtctattccccccgaggtcaagttcaacaaaccctttgtcttcttaatgattgaacaaaataccaagtctcccctcttcatgggaaaagtggtgaatcccacccagaaataactgcctgtcactcctcagcccctcccctccatccctggccccctccctgaatgacattaaagaagggttgagctggtccctgcctgcgtgtgtgactgcaaac

SEQ ID NO: 15 - DNA, 3220 bp, Homo sapiens
  source 1..3220: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgt

SEQ ID NO: 16 - DNA, 3199 bp, Homo sapiens
  source 1..3199: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 17 - DNA, 3513 bp, Homo sapiens
  source 1..3513: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggctgcgaaaggagtcattgtacctggctcagaaaccacagcgtcctgtgtccaaggtggagggggtggcgtgagtcagacagtctctgggagagtaccacttagctggccctctgctctcactgcagaatccttagtggctgttccactggtagcaagatctaccatttactgagtcaccccaaaatgcctgatgctgaagacttactgccgccctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 18 - DNA, 3236 bp, Homo sapiens
  source 1..3236: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcctgtggaactgagtgagcagcagcagcaatgtcccacctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 19 - DNA, 3532 bp, Homo sapiens
  source 1..3532: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggctgctggctcagaaaccacagcgtcctgtgtccaaggtggagggggtggcgtgagtcagacagtctctgggagagtaccacttagctggccctctgctctcactgcagaatccttagtggctgttccactggtagcaagatctaccatttactgagtcaccccaaaatgcctgatgctgaagacttactgccgccctgtggaactgagtgagcagcagcagcaatgtcccacctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 20 - DNA, 3340 bp, Homo sapiens
  source 1..3340: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggctgctgtggaactgagtgagcagcagcagcaatgtcccacctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 21 - DNA, 3495 bp, Homo sapiens
  source 1..3495: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggctgctggctcagaaaccacagcgtcctgtgtccaaggtggagggggtggcgtgagtcagacagtctctgggagagtaccacttagctggccctctgctctcactgcagaatccttagtggctgttccactggtagcaagatctaccatttactgagtcaccccaaaatgcctgatgctgaagacttactgccgccctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 22 - DNA, 3492 bp, Homo sapiens
  source 1..3492: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggctggctcagaaaccacagcgtcctgtgtccaaggtggagggggtggcgtgagtcagacagtctctgggagagtaccacttagctggccctctgctctcactgcagaatccttagtggctgttccactggtagcaagatctaccatttactgagtcaccccaaaatgcctgatgctgaagacttactgccgccctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 23 - DNA, 3510 bp, Homo sapiens
  source 1..3510: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggcgaaaggagtcattgtacctggctcagaaaccacagcgtcctgtgtccaaggtggagggggtggcgtgagtcagacagtctctgggagagtaccacttagctggccctctgctctcactgcagaatccttagtggctgttccactggtagcaagatctaccatttactgagtcaccccaaaatgcctgatgctgaagacttactgccgccctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 24 - DNA, 3303 bp, Homo sapiens
  source 1..3303: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggctgctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 25 - DNA, 3300 bp, Homo sapiens
  source 1..3300: mol_type = genomic DNA
tgcaagaaatgtagttctatttattctctgttctaatgggtataaacattttgttatctaacttgaacatcataccaactcaaaggcaggtcttactagcaatgactggggctcagagaggtttggcgacttcacgaaggtcacacagctgtcagggggaaaagtcagaacttggatccaggtcttcagactctcaggtctggtgtcatcctagggggcttggtgatggccatatctttaatgtatttgtggagagtgaaaggctgtcagtgagtaagcttaagagaacaggagacttgtgtgggaaacagtcggtatccattgattagactgaatcatgtagaattgctaatttcaccattttgaactatcgaaatcactatttggtatgactcaacctcatcctttaagtacacattcatgacagtgagtggttagacagtgattcctagattagtttgggatggggcagtgccttccataggacaaggccattcctggtagagacggagggagcaggctgtccttcagctaggggcccaggggacttcctggctgctgtgtcctttagcagggccttggaggatggaaggactctcctggcccttaccacaggggctattcaggaacagcctcctgccgtggcactggagctgcggtggccctctgaagactgcagggacagcaacaggcacaaagaagtcaggctgcatgtggccccagtcgggactcagaggaggaaagggaggggttgcgggggtcaatgggtgatgtgcttcctctcccatagctgaggagtccttgcaatggccttcctgagccatcagcaggcctatggccatgtgactagggaggagaagggatatagggtaatggtcttctggggcctgctgggcctggtcaagtctgaggaggagatagagaaaacaaagcagagaccctcctcttcatctggggagaagggacctgattctaaacggagatatgtgaggctttctggggcagcgatggaaggacaaggacagaataggtgtccttgttgccccatggagaatgggcttcaggaagaatctgcctcagtcattttccagaagtgcctgagaggtgcagggcccgagtctggttaggtgacagcgggtcaagaggaggacattgtcctctctcttgtgttgctgcagatgccacaaaactgggccacggcacgatcaggcagttctggggcccccaggagggcagccttggggtgggcacagccagccctggcaggatgagcaactctgggtgggggggagtgggggatgagcagggggacatgaagatgcttggtggagcctggggtcatggctggtatctggttcctcccctgtgattccttcttggggactccaagacaggacaaggaagactggagccctccagaaacagatgggcccaggtccgtaagctgaggattcagtcccccctggattcaagcccagcatgtgcctacccagccagatgctccatgaacacagttcagggggcccgaagacagcactgttacctggagcccacatacagcctcagcaggcaaagggagactcagagaaaacatgggagggatttacagtcacatgcaggcagggaccagctcaacccttctttaatgtcatccagggagggggccagggatggaggggaggggttgaggagcgagaggcagttatttttgggtgggattcaccacttttcccatgaagaggggagacttggtattttgttcaatcattaagaagacaaagggtttgttgaacttgacctcgggggggatagacatgggtatggcctctaaaaacatggccccagcagcttcagtccctttctcgtcgatggtcagcacagccttatgcacggccttggagagcttcaggggtgcctcctctgtgaccccggagaggtcagccccattgctgaagaccttagtgatgcccagttgacccaggacgctcttcagatcataggttccagtaatggacagtttgggtaaatgtaagctggcagaccttctgtcttcattttccaggaacttggtgatgatatcgtgggtgagttcattttccaggtgctgtagtttcccctcatcaggcaggaagaagatggcggtggcattgcccaggtatttcatcagcagcacccagctggacagcttcttacagtgctggatgttaaacatgcctaaacgcttcatcataggcaccttcacggtggtcacctggtccacgtggaagtcctcttcctcggtgtccttgacttcaaagggtctctcccatttgcctttaaagaagatgtaattcaccagagcaaaaactgtgtctctgtcaagctccttgaccaaatccacaattttcccttgagtacccttctccacgtaatcgttgatctgtttcttggcctcttcggtgtccccgaagttgacagtgaaggcttctgagtggtacaactttttaacatcctccaaaaacttatccactagcttcaggccctcgctgaggaacaggccattgccggtggtcagctggagctggctgtctggctggttgagggtacggaggagttcctggaagccttcatggatctgagcctccggaatctccgtgaggttgaaattcaggccctccaggatttcatcgtgagtgtcagccttggtccccagggagagcattgcaaaggctgtagcgatgctcactggggagaagaagatattggtgctgttggactggtgtgccagctggcggtataggctgaaggcgaactcagccaggttgggggtgatcttgttgaaggttgggtgatcctgatcatggtgggatgtatctgtcttctgggcagcatctccctggggatcctcagccagggagacagggaccaggcagcacaggcctgccagcaggaggatgccccacgagacagaagacggcattgtcgattcactgtcccaggtcagtggtggtgcctgaagctgaggagacagggccctgtcctcgtccgtatttaagcagtggatccagaggggcaacgggggaggctttcctgctctcctcaagctctcctcaagctctgtctcttctggcaggcacaggagagtggcctgaaggctggcaggaggttgccgcccctccaacctggaattcctggcagcagcagcggctaggccttcctcggaggcccgaccccctcctccttcttggttcagctcaggactctgagggttgctgcgtggaggcagtgcatgccctgggcacagtgcccagttcctgccca

SEQ ID NO: 26 - DNA, 1321 bp, Macaca mulatta
  source 1..1321: mol_type = genomic DNA
atgtcattcagggagggggccagggatggaggggaggggctgaggagtgacaggcagttatttctgggtgggattcaccacttttcccatgaagaggggagacttggtattttgttcaatcattaagaagacaaagggtttgttgaacttgacctcggggggaatagacatgggtatggcctctaaaaacatggccccagcagcttcagtccctttctcatcgatggtcagcacagccttatgcacggccttggagagcttcaggggtgcctcctccgtgatccccgagaggtcagccccattgctgaagaccttagtgatacccaggtggcccaggactgtcttcagatcataggttccagtaatggccagtctgggtaaatgtaagttggcagacctgctgttttcattttccaggaacttggtgatgatatcatgggtgagttcattttccaggtgctgcagtttcccctcatcaggcaggaagaagatggcggtggcattgcccaggtatttcatcagcagcacccagctggacagcttctcacagtggtagatgttaaacatgcctaaacgcctcatcatgggcaccttcacggtggtcgcctggtccacgtggaagtcctcttccttggtggcctcaacgtcaaagggtctctcccatttgcctttaaagaagatgtaattcaccagagcaaaaactgtgtctctgtcaagctccttgaccaaatccacaattttcccttgagtttccttctccacgtaattgttgatctgtttcttggcctcttcggtgtcctcaaagttgacagagaaggcttctgagtggtacagttttttgacatcctccaaaaacttatccactaccttcaggctcttgttgaggaacaggccgttgccggtggtcagctggagctggctgtctggcttgttgagggtatggaggagttcctggaagccttcatggacctgagcctccggaatctccgtgacgttgaaattcaggccctccaggatttcactgtgagtgtcagccttggtccccagggagagcattgcaaaggctgtagcgatgctcactggggagaagaagatattggtgctgttggactggtgtgccagctggcggtataggctgaagccgaactcagccaggctgggggtgatcttgttgagggttgggtggtcctgatcatggtgggatgtatccgtcttctgggcagcatctccctggggatcctcagccagagagccggggagcaggcagcacaggcctgccagcaggaggacgccccatgagacagaagatggcattgtcctg

SEQ ID NO: 27 - DNA, 2064 bp, Macaca mulatta
  source 1..2064: mol_type = genomic DNA
gtttgcagtcacacacgcaggcagggaccagctcaacccttctttaatgtcattcagggagggggccagggatggaggggaggggctgaggagtgacaggcagttatttctgggtgggattcaccacttttcccatgaagaggggagacttggtattttgttcaatcattaagaagacaaagggtttgttgaacttgacctcggggggaatagacatgggtatggcctctaaaaacatggccccagcagcttcagtccctttctcatcgatggtcagcacagccttatgcacggccttggagagcttcaggggtgcctcctccgtgatccccgagaggtcagccccattgctgaagaccttagtgatacccaggtggcccaggactgtcttcagatcataggttccagtaatggccagtctgggtaaatgtaagttggcagacctgctgttttcattttccaggaacttggtgatgatatcatgggtgagttcattttccaggtgctgcagtttcccctcatcaggcaggaagaagatggcggtggcattgcccaggtatttcatcagcagcacccagctggacagcttctcacagtggtagatgttaaacatgcctaaacgcctcatcatgggcaccttcacggtggtcgcctggtccacgtggaagtcctcttccttggtggcctcaacgtcaaagggtctctcccatttgcctttaaagaagatgtaattcaccagagcaaaaactgtgtctctgtcaagctccttgaccaaatccacaattttcccttgagtttccttctccacgtaattgttgatctgtttcttggcctcttcggtgtcctcaaagttgacagagaaggcttctgagtggtacagttttttgacatcctccaaaaacttatccactaccttcaggctcttgttgaggaacaggccgttgccggtggtcagctggagctggctgtctggcttgttgagggtatggaggagttcctggaagccttcatggacctgagcctccggaatctccgtgacgttgaaattcaggccctccaggatttcactgtgagtgtcagccttggtccccagggagagcattgcaaaggctgtagcgatgctcactggggagaagaagatattggtgctgttggactggtgtgccagctggcggtataggctgaagccgaactcagccaggctgggggtgatcttgttgagggttgggtggtcctgatcatggtgggatgtatccgtcttctgggcagcatctccctggggatcctcagccagagagccggggagcaggcagcacaggcctgccagcaggaggacgccccatgagacagaagatggcattgtcgattcaccgtcccaggtcagtggtggtgcctgaggctgaggagacagagccctgtccttgtccgtatttaagcagtgggtgcagaggggcaacgggggaggctgctggtgaatattaaccaagatcaccccagttaccggaggagcaaacagggactaagttcacaggctgggcgctgagtcgcccgcccacgctgtccggacgctctgcctgagcagcatacagcctccactgcacgtaccaaaaggagtcattgtacctggctcagaaaccacagtgtcctgcgtccaaggtggagggggtggcgtgagtcagccagtcgctgggagagtaccactttgctggccctctgctctcactgcagaatccttagcggctgttccactggtagcaagatctaccatttactaattcaccccgaaatgcctgatgctgaagactgatgccgccctggaattcctggcagcagcagtggctaggccttcctcagagtgctgatcccctcctcctcctttgctcagctcagtactctgagggttgctgcgtggaggcagtgcacgccctgggcacagggcccagttcctgcccacccaggaagttggcctcgggtggcgggtggcggaggcaataggttggggaggggcggggagcttgggcaggaaggggccttgcccattgccaggcagacacaagactgggc

SEQ ID NO: 28 - DNA, 1608 bp, Macaca mulatta
  source 1..1608: mol_type = genomic DNA
gtttgcagtcacacacgcaggcagggaccagctcaacccttctttaatgtcattcagggagggggccagggatggaggggaggggctgaggagtgacaggcagttatttctgggtgggattcaccacttttcccatgaagaggggagacttggtattttgttcaatcattaagaagacaaagggtttgttgaacttgacctcggggggaatagacatgggtatggcctctaaaaacatggccccagcagcttcagtccctttctcatcgatggtcagcacagccttatgcacggccttggagagcttcaggggtgcctcctccgtgatccccgagaggtcagccccattgctgaagaccttagtgatacccaggtggcccaggactgtcttcagatcataggttccagtaatggccagtctgggtaaatgtaagttggcagacctgctgttttcattttccaggaacttggtgatgatatcatgggtgagttcattttccaggtgctgcagtttcccctcatcaggcaggaagaagatggcggtggcattgcccaggtatttcatcagcagcacccagctggacagcttctcacagtggtagatgttaaacatgcctaaacgcctcatcatgggcaccttcacggtggtcgcctggtccacgtggaagtcctcttccttggtggcctcaacgtcaaagggtctctcccatttgcctttaaagaagatgtaattcaccagagcaaaaactgtgtctctgtcaagctccttgaccaaatccacaattttcccttgagtttccttctccacgtaattgttgatctgtttcttggcctcttcggtgtcctcaaagttgacagagaaggcttctgagtggtacagttttttgacatcctccaaaaacttatccactaccttcaggctcttgttgaggaacaggccgttgccggtggtcagctggagctggctgtctggcttgttgagggtatggaggagttcctggaagccttcatggacctgagcctccggaatctccgtgacgttgaaattcaggccctccaggatttcactgtgagtgtcagccttggtccccagggagagcattgcaaaggctgtagcgatgctcactggggagaagaagatattggtgctgttggactggtgtgccagctggcggtataggctgaagccgaactcagccaggctgggggtgatcttgttgagggttgggtggtcctgatcatggtgggatgtatccgtcttctgggcagcatctccctggggatcctcagccagagagccggggagcaggcagcacaggcctgccagcaggaggacgccccatgagacagaagatggcattgtcctggaattcctggcagcagcagtggctaggccttcctcagagtgctgatcccctcctcctcctttgctcagctcagtactctgagggttgctgcgtggaggcagtgcacgccctgggcacagggcccagttcctgcccacccaggaagttggcctcgggtggcgggtggcggaggcaataggttggggaggggcggggagcttgggcaggaaggggccttgcccattgccaggcagacacaaga

SEQ ID NO: 29 - protein, 16 aa, unidentified
  REGION 1..16: note = Description of Unknown: RFGF peptide
AAVALLPAVLLALLAP

SEQ ID NO: 30 - protein, 11 aa, unidentified
  REGION 1..11: note = Description of Unknown: RFGF analogue peptide
AALLPVLLAAP

SEQ ID NO: 31 - protein, 13 aa, Human immunodeficiency virus
GRKKRRQRRRPPQ

SEQ ID NO: 32 - protein, 16 aa, Drosophila sp.
RQIKIWFQNRRMKWKK

SEQ ID NO: 33 - RNA, 21 nt, synthetic construct
  misc_feature 1..21: note = Description of Artificial Sequence: Synthetic oligonucleotide
  modified_base 1: mod_base = OTHER; note = 2'-O-methyl modification and phosphorothioate linkage
  modified_base 2: mod_base = OTHER; note = 2'-O-methyl modification and phosphorothioate linkage
  modified_base 3: mod_base = OTHER; note = 2'-O-methyl modification
  modified_base 4: mod_base = OTHER; note = 2'-O-methyl modification
  modified_base 5: mod_base = OTHER; note = 2'-O-methyl modification
  modified_base 6: mod_base = OTHER; note = 2'-O-methyl modification
  modified_base 7: mod_base = OTHER; note = 2'-fluoro modification
  modified_base 8: mod_base = OTHER; note = 2'-O-methyl modification
  modified_base 9: mod_base = OTHER; note = 2'-fluoro modification
  modified_base 10: mod_base = OTHER; note = 2'-fluoro modification
  modified_base 11: mod_base = OTHER; note = 2'-fluoro modification
  modified_base 12: mod_base = OTHER; note = 2'-O-methyl modification
  modified_base 13: mod_base = OTHER; note = 2'-O-methyl modification
  modified_base 14: mod_base = OTHER; note = 2'-O-methyl modification
  modified_base 15: mod_base = OTHER; note = 2'-O-methyl modification
  modified_base 16: mod_base = OTHER; note = 2'-O-methyl modification
  modified_base 17: mod_base = OTHER; note = 2'-O-methyl modification
  modified_base 18: mod_base = OTHER; note = 2'-O-methyl modification
  modified_base 19: mod_base = OTHER; note = 2'-O-methyl modification
  modified_base 20: mod_base = OTHER; note = 2'-O-methyl modification
  modified_base 21: mod_base = OTHER; note = 2'-O-methyl modification
  source 1..21: mol_type = other RNA
cttcttaatgattgaacaaaa

SEQ ID NO: 34 - DNA, 23 bp, synthetic construct
  misc_feature 1..23: note = Synthetic oligonucleotide
  modified_base 1: mod_base = OTHER; note = 2'-O-methyl uracil and phosphorothioate linkage
  modified_base 2: mod_base = OTHER; note = 2'-fluoro uracil and phosphorothioate linkage
  modified_base 3: mod_base = OTHER; note = 2'-O-methyl uracil
  modified_base 4: mod_base = OTHER; note = 2'-O-methyl uracil
  modified_base 5: mod_base = OTHER; note = 2'-O-methyl modification
  modified_base 6: mod_base = OTHER; note = 2'-O-methyl uracil
  modified_base 7: mod_base = OTHER; note = thymidine-glycol nucleic acid (GNA) S-Isomer
  modified_base 8: mod_base = OTHER; note = 2'-O-methyl modification
  modified_base 9: mod_base = OTHER; note = 2'-O-methyl modification
  modified_base 10: mod_base = OTHER; note = 2'-O-methyl modification
  modified_base 11: mod_base = OTHER; note = 2'-O-methyl uracil
  modified_base 12: mod_base = OTHER; note = 2'-O-methyl modification
  modified_base 13: mod_base = OTHER; note = 2'-O-methyl modification
  modified_base 14: mod_base = OTHER; note = 2'-fluoro uracil
  modified_base 15: mod_base = OTHER; note = 2'-O-methyl uracil
  modified_base 16: mod_base = OTHER; note = 2'-fluoro modification
  modified_base 17: mod_base = OTHER; note = 2'-O-methyl modification
  modified_base 18: mod_base = OTHER; note = 2'-O-methyl modification
  modified_base 19: mod_base = OTHER; note = 2'-O-methyl modification
  modified_base 20: mod_base = OTHER; note = 2'-O-methyl modification
  modified_base 21: mod_base = OTHER; note = 2'-O-methyl modification and phosphorothioate linkage
  modified_base 22: mod_base = OTHER; note = 2'-O-methyl modification and phosphorothioate linkage
  modified_base 23: mod_base = OTHER; note = 2'-O-methyl modification
  source 1..23: mol_type = other DNA
ttttgttcaatcattaagaagac

SEQ ID NO: 35 - RNA, 21 nt, synthetic construct
  misc_feature 1..21: note = Description of Artificial Sequence: Synthetic oligonucleotide
  modified_base 1: mod_base = OTHER; note = 2'-O-methyl modification and phosphorothioate linkage
  modified_base 2: mod_base = OTHER; note = 2'-O-methyl modification and phosphorothioate linkage
  modified_base 3: mod_base = OTHER; note = 2'-O-methyl modification
  modified_base 4: mod_base = OTHER; note = 2'-O-methyl modification
  modified_base 5: mod_base = OTHER; note = 2'-O-methyl modification
  modified_base 6: mod_base = OTHER; note = 2'-O-methyl modification
  modified_base 7: mod_base = OTHER; note = 2'-fluoro modification
  modified_base 8: mod_base = OTHER; note = 2'-O-methyl modification
  modified_base 9: mod_base = OTHER; note = 2'-fluoro modification
  modified_base 10: mod_base = OTHER; note = 2'-fluoro modification
  modified_base 11: mod_base = OTHER; note = 2'-fluoro modification
  modified_base 12: mod_base = OTHER; note = 2'-O-methyl modification
  modified_base 13: mod_base = OTHER; note = 2'-O-methyl modification
  modified_base 14: mod_base = OTHER; note = 2'-O-methyl modification
  modified_base 15: mod_base = OTHER; note = 2'-O-methyl modification
  modified_base 16: mod_base = OTHER; note = 2'-O-methyl modification
  modified_base 17: mod_base = OTHER; note = 2'-O-methyl modification
  modified_base 18: mod_base = OTHER; note = 2'-O-methyl modification
  modified_base 19: mod_base = OTHER; note = 2'-O-methyl modification
  modified_base 20: mod_base = OTHER; note = 2'-O-methyl modification
  modified_base 21: mod_base = OTHER; note = N-[tris(GalNAc-alkyl)-amidodecanoyl)]-4-hydroxyprol inol
  source 1..21: mol_type = other RNA
cttcttaatgattgaacaaaa

SEQ ID NO: 36 - RNA, 21 nt, synthetic construct
  misc_feature 1..21: note = Description of Artificial Sequence: Synthetic oligonucleotide
  source 1..21: mol_type = other RNA
cttcttaatgattgaacaaaa

SEQ ID NO: 37 - RNA, 23 nt, synthetic construct
  misc_feature 1..23: note = Description of Artificial Sequence: Synthetic oligonucleotide
  source 1..23: mol_type = other RNA
ttttgttcaatcattaagaagac

SEQ ID NO: 38 - DNA, 23 bp, synthetic construct
  misc_feature 1..23: note = Synthetic oligonucleotide
  modified_base 1..4: mod_base = OTHER; note = uracil
  modified_base 7: mod_base = OTHER; note = uracil
  modified_base 11: mod_base = OTHER; note = uracil
  modified_base 14..15: mod_base = OTHER; note = uracil
  source 1..23: mol_type = other DNA
ttttgttcaatcattaagaagac

SEQ ID NO: 39 - DNA, 23 bp, synthetic construct
  misc_feature 1..23: note = Synthetic oligonucleotide
  modified_base 1..4: mod_base = OTHER; note = uracil
  modified_base 6: mod_base = OTHER; note = uracil
  modified_base 11: mod_base = OTHER; note = uracil
  modified_base 14..15: mod_base = OTHER; note = uracil
  source 1..23: mol_type = other DNA
ttttgttcaatcattaagaagac

SEQ ID NO: 40-416 - skipped (377 entries)

SEQ ID NO: 417 - RNA, 21 nt, synthetic construct
  misc_feature 1..21: note = Description of Artificial Sequence: Synthetic oligonucleotide
  source 1..21: mol_type = other RNA
cttcttaatgattgaacaaaa

SEQ ID NO: 418 - RNA, 21 nt, synthetic construct
  misc_feature 1..21: note = Description of Artificial Sequence: Synthetic oligonucleotide
  modified_base 1: mod_base = OTHER; note = 2'-O-methyl modification and phosphorothioate linkage
  modified_base 2: mod_base = OTHER; note = 2'-O-methyl modification and phosphorothioate linkage
  modified_base 3: mod_base = OTHER; note = 2'-O-methyl modification
  modified_base 4: mod_base = OTHER; note = 2'-O-methyl modification
  modified_base 5: mod_base = OTHER; note = 2'-O-methyl modification
  modified_base 6: mod_base = OTHER; note = 2'-O-methyl modification
  modified_base 7: mod_base = OTHER; note = 2'-O-methyl modification
  modified_base 8: mod_base = OTHER; note = 2'-O-methyl modification
  modified_base 9: mod_base = OTHER; note = 2'-O-methyl modification
  modified_base 10: mod_base = OTHER; note = 2'-fluoro modification
  modified_base 11: mod_base = OTHER; note = 2'-fluoro modification
  modified_base 12: mod_base = OTHER; note = 2'-O-methyl modification
  modified_base 13: mod_base = OTHER; note = 2'-O-methyl modification
  modified_base 14: mod_base = OTHER; note = 2'-O-methyl modification
  modified_base 15: mod_base = OTHER; note = 2'-O-methyl modification
  modified_base 16: mod_base = OTHER; note = 2'-O-methyl modification
  modified_base 17: mod_base = OTHER; note = 2'-O-methyl modification
  modified_base 18: mod_base = OTHER; note = 2'-O-methyl modification
  modified_base 19: mod_base = OTHER; note = 2'-O-methyl modification
  modified_base 20: mod_base = OTHER; note = 2'-O-methyl modification
  modified_base 21: mod_base = OTHER; note = N-[tris(GalNAc-alkyl)-amidodecanoyl)]-4-hydroxyprol inol
  source 1..21: mol_type = other RNA
cttcttaatgattgaacaaaa

SEQ ID NO: 419 - RNA, 23 nt, synthetic construct
  misc_feature 1..23: note = Description of Artificial Sequence: Synthetic oligonucleotide
  source 1..23: mol_type = other RNA
ttttgttcaatcattaagaagac

SEQ ID NO: 420 - RNA, 23 nt, synthetic construct
  misc_feature 1..23: note = Description of Artificial Sequence: Synthetic oligonucleotide
  modified_base 1: mod_base = OTHER; note = 2'-O-methyl modification and phosphorothioate linkage
  modified_base 2: mod_base = OTHER; note = 2'-fluoro modification and phosphorothioate linkage
  modified_base 3: mod_base = OTHER; note = 2'-O-methyl modification
  modified_base 4: mod_base = OTHER; note = 2'-fluoro modification
  modified_base 5: mod_base = OTHER; note = 2'-O-methyl modification
  modified_base 6: mod_base = OTHER; note = 2'-fluoro modification
  modified_base 7: mod_base = OTHER; note = 2'-O-methyl modification
  modified_base 8: mod_base = OTHER; note = 2'-fluoro modification
  modified_base 9: mod_base = OTHER; note = 2'-O-methyl modification
  modified_base 10: mod_base = OTHER; note = 2'-fluoro modification
  modified_base 11: mod_base = OTHER; note = 2'-O-methyl modification
  modified_base 12: mod_base = OTHER; note = 2'-O-methyl modification
  modified_base 13: mod_base = OTHER; note = 2'-O-methyl modification
  modified_base 14: mod_base = OTHER; note = 2'-fluoro modification
  modified_base 15: mod_base = OTHER; note = 2'-O-methyl modification
  modified_base 16: mod_base = OTHER; note = 2'-fluoro modification
  modified_base 17: mod_base = OTHER; note = 2'-O-methyl modification
  modified_base 18: mod_base = OTHER; note = 2'-fluoro modification
  modified_base 19: mod_base = OTHER; note = 2'-O-methyl modification
  modified_base 20: mod_base = OTHER; note = 2'-fluoro modification
  modified_base 21: mod_base = OTHER; note = 2'-O-methyl modification and phosphorothioate linkage
  modified_base 22: mod_base = OTHER; note = 2'-O-methyl modification and phosphorothioate linkage
  modified_base 23: mod_base = OTHER; note = 2'-O-methyl modification
  source 1..23: mol_type = other RNA
ttttgttcaatcattaagaagac

(19) United States
(12) Patent Application Publication
(10) Pub. No.: US 2025/0043279 A1
(43) Pub. Date: Feb. 6, 2025

(54) SERPINA1 iRNA COMPOSITIONS AND METHODS OF USE THEREOF

(71) Applicant: Alnylam Pharmaceuticals, Inc., Cambridge, MA (US)

(72) Inventors: Mark K. Schlegel, Boston, MA (US); Maja Janas, Cambridge, MA (US); Vasant R. Jadhav, Sharon, MA (US); Donald Foster, Attleboro, MA (US); Muthiah Manoharan, Weston, MA (US); Kallanthottathil G. Rajeev, Wayland, MA (US); Alexander V. Kel'in, Swampscott, MA (US); Klaus Charisse, Acton, MA (US); Jayaprakash K. Nair, Cambridge, MA (US); Martin A. Maier, Belmont, MA (US); Shigeo Matsuda, Cambridge, MA (US); Muthusamy Jayaraman, Cambridge, MA (US); Alfica Sehgal, Medford, MA (US); Christopher Brown, Cambridge, MA (US); Kevin Fitzgerald, Brookline, MA (US); Stuart Milstein, Arlington, MA (US)

(21) Appl. No.: 18/644,183

(22) Filed: Apr. 24, 2024

Related U.S. Application Data
(63) Continuation of application No. 18/213,917, filed on Jun. 26, 2023, now abandoned.
Application No. 18/213,917 is a continuation of application No. 17/194,431, filed on Mar. 8, 2021, now Pat. No. 11,725,207.
Application No. 17/194,431 is a division of application No. 16/420,263, filed on May 23, 2019, now Pat. No. 10,995,336.
Application No. 16/420,263 is a continuation of application No. PCT/US2017/062701, filed on Nov. 21, 2017.
(60) Provisional application No. 62/561,514, filed on Sep. 21, 2017.
(60) Provisional application No. 62/549,099, filed on Aug. 23, 2017.
(60) Provisional application No. 62/548,589, filed on Aug. 22, 2017.
(60) Provisional application No. 62/425,907, filed on Nov. 23, 2016.

(51) Int. Cl. C12N 15/113

(52) U.S. Cl. CPC C12N 15/113; C12N 2310/14; C12N 2310/315; C12N 2310/351

(57) ABSTRACT

The invention relates to RNAi agents, e.g., double stranded RNAi agents, targeting the Serpina1 gene, and methods of using such RNAi agents to inhibit expression of Serpina1 and methods of treating subjects having a Serpina1 associated disease, such as a liver disorder.

### RELATED APPLICATIONS

This application is a continuation of U.S. patent application Ser. No. 18/213,917, filed on Jun. 26, 2023, which is a continuation of U.S. patent application Ser. No. 17/194,431, filed on Mar. 8, 2021, now, U.S. Pat. No. 11,725,207, issued on Aug. 15, 2023, which is a divisional of Ser. No. 16/420,263, filed on May 23, 2019, now U.S. Pat. No. 10,995,336, issued on May 4, 2021, which is a 35 § U.S.C. 111(a) continuation application which claims the benefit of priority to PCT/US2017/062701, filed on Nov. 21, 2017, which claims the benefit of priority to U.S. Provisional Application No. 62/425,907, filed on Nov. 23, 2016; U.S. Provisional Application No. 62/548,589 filed on Aug. 22, 2017; U.S. Provisional Application No. 62/549,099 filed on Aug. 23, 2017; and U.S. Provisional Application No. 62/561,514 filed on Sep. 21, 2017. The entire contents of each of the foregoing applications are hereby incorporated herein by reference.

This application is related to U.S. Provisional Application No. 61/826,125, filed on May 22, 2013, U.S. Provisional Application No. 61/898,695, filed on Nov. 1, 2013; U.S. Provisional Application No. 61/979,727, filed on Apr. 15, 2014; U.S. Provisional Application No. 61/989,028, filed on May 6, 2014; U.S. patent application Ser. No. 14/284,745, now U.S. Pat. No. 9,574,192, issued on Feb. 21, 2017; U.S. patent application Ser. No. 15/399,820, filed on Jan. 6, 2017, now U.S. Pat. No. 10,030,244, issued on Jul. 24, 2018; U.S. patent application Ser. No. 16/008,461, filed on Jun. 14, 2018, now U.S. Pat. No. 10,920,223, issued on Feb. 16, 2021; U.S. patent application Ser. No. 16/679,691, now U.S. Pat. No. 10,683,504, issued on Jun. 16, 2020; U.S. patent application Ser. No. 17/141,275, filed on Jan. 5, 2021; and International Patent Application No. PCT/US2014/039109, filed on May 22, 2014. The entire contents of each of the foregoing applications are hereby incorporated herein by reference.

### SEQUENCE LISTING

The application contains a Sequence Listing which has been submitted electronically in .XML format and is hereby incorporated by reference in its entirety. Said .XML copy, created on Oct. 7, 2024, is named “121301_07805_SL.xml” and is 268,003 bytes in size. The sequence listing contained in this .XML file is part of the specification and is hereby incorporated by reference herein in its entirety.

### BACKGROUND OF THE INVENTION

Serpina1 encodes alpha-1-antitrypsin which predominantly complexes with and inhibits the activity of neutrophil elastase produced by hepatocytes, mononuclear monocytes, alveolar macrophages, enterocytes, and myeloid cells. Subjects having variations in one or both copies of the Serpina1 gene may suffer from alpha-1-antitrypsin deficiency and are at risk of developing pulmonary emphysema and/or chronic liver disease due to greater than normal elastase activity in the lungs and liver.

In affected subjects, the deficiency in alpha-1-antitrypsin is a deficiency of wild-type, functional alpha-1-antitrypsin. In some cases, a subject having a variation in one or both copies of the Serpina1 gene is carrying a null allele. In other cases, a subject having a variation in one or both copies of the Serpina1 gene is carrying a deficient allele.

For example, a subject having a deficient allele of Serpina1, such as the PIZ allele, may be producing misfolded proteins which cannot be properly transported from the site of synthesis to the site of action within the body. Such subjects are typically at risk of developing lung and/or liver disease. Subjects having a Serpina1 null allele, such as the PINULL (Granite Falls), are typically only at risk of developing lung disease.

Liver disease resulting from alpha-1 antitrypsin deficiency is the result of variant forms of alpha-1-antitypsin produced in liver cells which misfold and are, thus, not readily transported out of the cells. This leads to a buildup of misfolded protein in the liver cells and can cause one or more diseases or disorders of the liver including, but not limited to, chronic liver disease, liver inflammation, cirrhosis, liver fibrosis, and/or hepatocellular carcinoma.

There are currently very limited options for the treatment of patients with liver disease arising from alpha-1-antitrypsin deficiency, including hepatitis vaccination, supportive care, and avoidance of injurious agents (e.g., alcohol and NSAIDs). Although replacement alpha-1-antitrypsin therapy is available, such treatment has no impact liver disease in these subjects and, although liver transplantation may be effective, it is a difficult, expensive and risky procedure and liver organs are not readily available.

Accordingly, there is a need in the art for effective treatments for Serpina1-associated diseases, such as a chronic liver disease, liver inflammation, cirrhosis, liver fibrosis, and/or hepatocellular carcinoma.

### SUMMARY OF THE INVENTION

As described in more detail below, disclosed herein are compositions comprising agents, e.g., RNAi agents, e.g., double stranded iRNA agents, targeting Serpina1. Also disclosed are methods using the compositions of the invention for inhibiting Serpina1 expression and for treating Serpina1 associated diseases, e.g., chronic liver disease, liver inflammation, cirrhosis, liver fibrosis, and/or hepatocellular carcinoma.

The present invention is based, at least in part on the discovery of effective nucleotide or chemical motifs for dsRNA agents targeting Serpina1 which are advantageous for inhibition of target gene expression, while having reduced off-target gene silencing effects, as well as compositions comprising such agents suitable for therapeutic use. More specifically, it has been discovered inter alia that dsRNA agents targeting Serpina1 where the antisense strand comprises at least one thermally destabilizing modification of the duplex within the seed region (i.e., at position 2-9 of the 5′-end of the antisense strand, counting from the 5′-end) and/or the dsRNA agent has a melting temperature in the range of from about 40° C. to about 80° C. can be more effective in mediating RNA interference than a parent dsRNA agent lacking the destabilizing modification.

Accordingly, in one aspect, the present invention provides a double stranded RNA (dsRNA) agent that inhibits expression of a serine peptidase inhibitor, clade A, member 1 (Serpina1) target gene sequence, comprising a sense strand and an antisense strand, wherein the antisense strand has sufficient complementarity to the target sequence to mediate RNA interference, wherein said antisense strand comprises at least one thermally destabilizing modification within the first 9 nucleotide positions of the 5′ region or a precursor thereof, wherein said sense strand comprises an asialoglycoprotein receptor (ASGPR) ligand, and wherein each of the sense strand and the antisense strand are independently 14 to 40 nucleotides in length.

In another aspect, the present invention provides a double stranded RNA (dsRNA) agent that inhibits expression of a serine peptidase inhibitor, clade A, member 1 (Serpina1) gene, comprising a sense strand and an antisense strand forming a double stranded region, wherein the sense strand comprises at least 15 contiguous nucleotides differing by no more than 3 nucleotides from the nucleotide sequence of SEQ ID NO:1, and the antisense strand comprises at least 15 contiguous nucleotides differing by no more than 3 nucleotides from the nucleotide sequence of SEQ ID NO:15, wherein said antisense strand comprises at least one thermally destabilizing modification of the double stranded region within the first 9 nucleotide positions of the 5′ region or a precursor thereof, wherein said sense strand comprises an asialoglycoprotein receptor (ASGPR) ligand, and wherein each of the sense strand and the antisense strand are independently 14 to 40 nucleotides in length.

In yet another aspect, the present invention provides a double stranded RNA (dsRNA) agent that inhibits expression of a serine peptidase inhibitor, clade A, member 1 (Serpina1) gene, comprising a sense strand and an antisense strand forming a double stranded region, said antisense strand comprising a region of complementarity to an mRNA encoding Serpina1, wherein the region of complementarity comprises at least 15 contiguous nucleotides differing by no more than 3 nucleotides from the nucleotide sequence of SEQ ID NO:15, wherein said antisense strand comprises at least one thermally destabilizing modification within the first 9 nucleotide positions of the 5′ region or a precursor thereof,
wherein said sense strand comprises an asialoglycoprotein receptor (ASGPR) ligand, wherein each of the sense strand and the antisense strand are independently 14 to 40 nucleotides in length.

In one embodiment, the dsRNA agent comprises at least four nucleotides comprising a 2′-fluoro modification.

In one embodiment, the dsRNA agent has the following characteristics: a) the thermally destabilizing modification is located in position 4-8 of the 5′ region of the antisense strand; b) and each of the sense and antisense strands independently comprise at least two nucleotides comprising a 2′-fluoro modification; and c) an ASGPR ligand attached to either end of the sense strand.

In another embodiment, the antisense strand has at least two of the following characteristics: a) the thermally destabilizing modification is located in position 4 to 8 of the antisense strand; b) at least two nucleotides comprise a 2′-fluoro modification; c) a phosphorothioate internucleotide linkages between nucleotide positions 1 and 2 (counting from the 5′ end); d) a length of 18 to 35 nucleotides.

In one embodiment, the sense strand has at least one of the following characteristics: a) the ASGPR ligand attached to either end of the sense strand; b) at least two nucleotides comprise a 2′-fluoro modification; c) the sense strand and the antisense strand form a double stranded region spanning at least 19 nucleotide positions and wherein the thermally destabilizing modification is located within said double stranded region.

In one embodiment, the thermally destabilizing modification is selected from the group consisting of

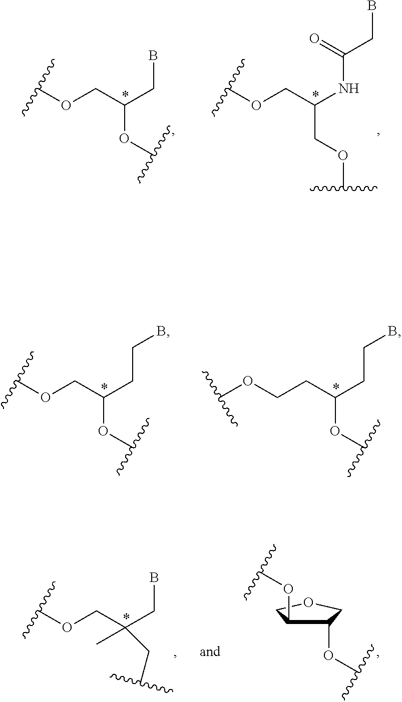

wherein B is nucleobase.

In one embodiment, the destabilizing modification is located in position 7 of the antisense strand.

In one embodiment, the ASGPR ligand is one or more GalNAc derivatives attached through a bivalent or trivalent branched linker.

In one embodiment, the ASGPR ligand is:

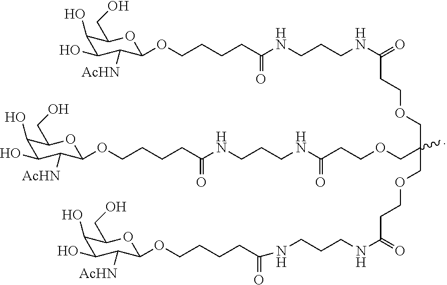 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the dsRNA agent is conjugated to the ligand as shown in the following schematic

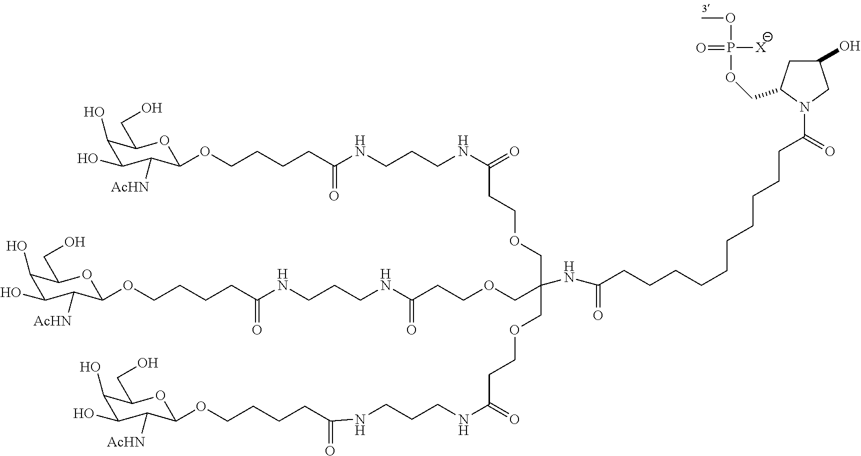

wherein X is O or S.

In one aspect, the present invention provides a double stranded RNA (dsRNA) agent that inhibits expression of a serine peptidase inhibitor, clade A, member 1 (Serpina1) target gene sequence, comprising a sense strand and an antisense strand, wherein the antisense strand has sufficient complementarity to a target sequence to mediate RNA interference, wherein the antisense strand comprises at least one thermally destabilizing modification within the first 9 nucleotide positions of the 5′ region, wherein each of the sense strand and the antisense strand are independently 14 to 40 nucleotides in length, and wherein the dsRNA has a melting temperature of from about 40° C. to about 80° C.

In another aspect, the present invention provides a double stranded RNA (dsRNA) agent that inhibits expression of a serine peptidase inhibitor, clade A, member 1 (Serpina1) target gene sequence, comprising a sense strand and an antisense strand, wherein the sense strand comprises at least 15 contiguous nucleotides differing by no more than 3 nucleotides from the nucleotide sequence of SEQ ID NO:1, and the antisense strand comprises at least 15 contiguous nucleotides differing by no more than 3 nucleotides from the nucleotide sequence of SEQ ID NO:15, wherein the antisense strand comprises at least one thermally destabilizing modification within the first 9 nucleotide positions of the 5′ region, wherein each of the sense strand and the antisense strand are independently 14 to 40 nucleotides in length, and wherein the dsRNA has a melting temperature of from about 40° C. to about 80° C.

In yet another aspect, the present invention provides a double stranded RNA (dsRNA) agent that inhibits expression of a serine peptidase inhibitor, clade A, member 1 (Serpina1) target gene sequence, comprising a sense strand and an antisense strand, said antisense strand comprising a region of complementarity to an mRNA encoding Serpina1, wherein the region of complementarity comprises at least 15 contiguous nucleotides differing by no more than 3 nucleotides from the nucleotide sequence of SEQ ID NO:15, wherein the antisense strand comprises at least one thermally destabilizing modification within the first 9 nucleotide positions of the 5′ region, wherein each of the sense strand and the antisense strand are independently 14 to 40 nucleotides in length, and wherein the dsRNA has a melting temperature of from about 40° C. to about 80° C.

In one embodiment, the dsRNA agent has a melting temperature of from about 55° C. to about 67° C. In another embodiment, the dsRNA agent has a melting temperature of from about 60° C. to about 67° C.

In one embodiment, the dsRNA agent further comprises an asialoglycoprotein receptor (ASGPR) ligand.

In one embodiment, the ASGPR ligand is one or more GalNAc derivatives attached through a bivalent or trivalent branched linker.

In one embodiment, the ASGPR ligand is:

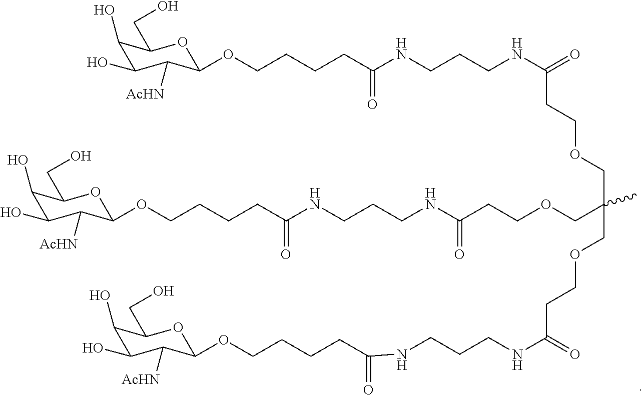 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the dsRNA agent is conjugated to the ligand as shown in the following schematic

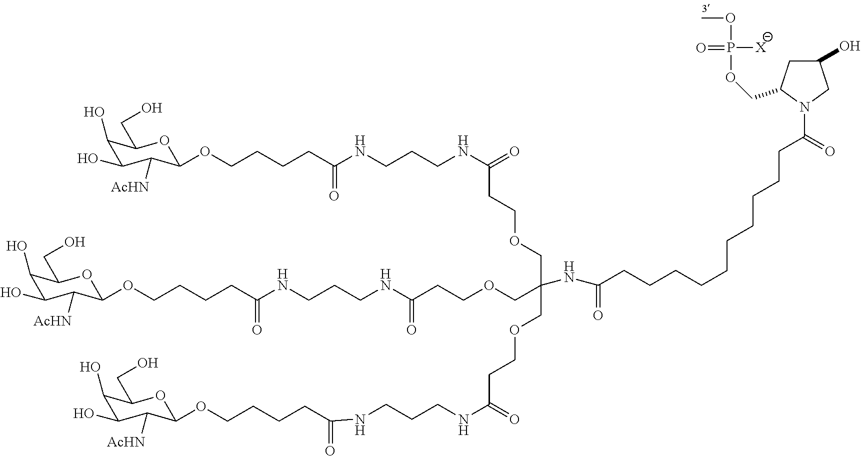

wherein X is O or S.

In one embodiment, the dsRNA agent further has at least one of the following characteristics: (i) the antisense strand comprises 2, 3, 4, 5 or 6 nucleotides comprising a 2′-fluoro modifications (ii) the antisense strand comprises 1, 2, 3 or 4 phosphorothioate internucleotide linkages; (iii) the sense strand is conjugated with a ligand; (iv) the sense strand comprises 2, 3, 4 or 5 nucleotides comprising a 2′-fluoro modification; (v) the sense strand comprises 1, 2, 3 or 4 phosphorothioate internucleotide linkages; (vi) the dsRNA comprises at least four nucleotides comprising a 2′-fluoro modification; (vii) the dsRNA comprises a double stranded region region of 12-40 nucleotide pairs in length; and (viii) a blunt end at 5′ end of the antisense strand.

In some embodiment, each strand of the dsRNA agent may have 15-30 nucleotides, 19-30 nucleotides; or the sense strand may have 21 nucleotides, and the antisense strand may have 23 nucleotides.

In one embodiment, the antisense strand comprises a region of complementarity comprising at least 15 contiguous nucleotides differing by no more than 3 nucleotides from nucleotides 1440-1480 of SEQ ID NO:1; or nucleotides 1441-1479 of SEQ ID NO:1; or nucleotides 1442-1478 of SEQ ID NO:1; or nucleotides 1443-1477 of SEQ ID NO:1; or nucleotides 1444-1476 of SEQ ID NO:1; or nucleotides 1445-1475 of SEQ ID NO:1; or nucleotides 1446-1474 of SEQ ID NO:1; or nucleotides 1447-1473 of SEQ ID NO:1; or nucleotides 1448-1473 of SEQ ID NO:1; or nucleotides 1448-1472 of SEQ ID NO:1; or nucleotides 1448-1471 of SEQ ID NO:1; or nucleotides 1448-1470 of SEQ ID NO:1; or nucleotides 1447-1469 of SEQ ID NO:1; or nucleotides 1446-1478 of SEQ ID NO:1; or nucleotides 1449-1471 of SEQ ID NO:1; or nucleotides 1450-1472 of SEQ ID NO:1; or nucleotides 1440-1475 of SEQ ID NO:1; or nucleotides 1445-1480 of SEQ ID NO:1; or nucleotides 1445-1475 of SEQ ID NO:1.

In one embodiment, the region of complemntarity comprises at least 15 contiguous nucleotides differing by no more than 3 nucleotides from the nucleotide sequence 5′-UUUUGUUCAAUCAUUAAGAAGAC-3′ (SEQ ID NO: 419).

In one embodiment, the sense strand comprises the nucleotide sequence 5′-CUUCUUAAUGAUUGAACAAAA-3′ (SEQ ID NO: 417) and the antisense strand comprises the nucleotide sequence 5′-UUUUGUUCAAUCAUUAAGAAGAC-3′ (SEQ ID NO: 419).

In one embodiment, the sense strand comprises the nucleotide sequence 5′-csusucuuAfaUfGfAfuugaacaaaa-3′ (SEQ ID NO: 33) and the antisense strand comprises the nucleotide sequence 5′-usUfsuugu(Tgn)caaucaUfuAfagaagsasc-3′ (SEQ ID NO: 34), wherein a, g, c, and u are 2′-O-methyl (2′-OMe) A, G, C, and U, respectively; Af, Gf, Cf and Uf are 2′-fluoro A, G, C and U, respectively; s is a phosphorothioate linkage; and (Tgn) is a thymidine-glycol nucleic acid (GNA)S-Isomer.

In one aspect, the present invention provides a double stranded RNA (dsRNA) molecule that inhibits expression of a serine peptidase inhibitor, clade A, member 1 (Serpina1) gene, comprising a sense strand and an antisense strand forming a double stranded region, wherein the sense strand comprises the nucleotide sequence 5′-csusucuuAfaUfGfAfuugaacaaaaL96-3′ (SEQ ID NO: 35) and the antisense strand comprises the nucleotide sequence 5′-usUfsuugu(Tgn)caaucaUfuAfagaagsasc-3′ (SEQ ID NO: 34), wherein a, g, c, and u are 2′-O-methyl (2′-OMe) A, G, C, and U, respectively; Af, Gf, Cf and Uf are 2′-fluoro A, G, C and U, respectively; s is a phosphorothioate linkage; (Tgn) is a thymidine-glycol nucleic acid (GNA)S-Isomer; and L96 is N-[tris(GalNAc-alkyl)-amidodecanoyl)]-4-hydroxyprolinol.

In one aspect the invention provides a dsRNA agent that inhibits expression of a serine peptidase inhibitor, clade a, member 1 (Serpina1) gene, comprising a sense strand and an antisense strand, each strand having 14 to 40 nucleotides, wherein the antisense strand has sufficient complementarity to a target Serpina1 sequence, e.g., nucleotides 1440-1480 of SEQ ID NO:1, to mediate RNA interference and wherein the antisense strand comprises at least one thermally destabilizing modification of the duplex within the seed region (i.e., at position 2-9 of the 5′-end of the antisense strand, counting from the 5′-end), and the dsRNA further has at least one (e.g., one, two, three, four, five, six seven, eight or all nine) of the following characteristics: (i) a melting temperature (Tₘ) of from about 40° C. to about 80° C.; (ii) the antisense comprises 2, 3, 4, 5 or 6 2′-fluoro modifications; (iii) the antisense comprises 1, 2, 3 or 4 phosphorothioate internucleotide linkages; (iv) the sense strand is conjugated with a ligand; (v) the sense strand comprises 2, 3, 4 or 5 2′-fluoro modifications; (vi) the sense strand comprises 1, 2, 3 or 4 phosphorothioate internucleotide linkages; (vii) the dsRNA comprises at least four 2′-fluoro modifications; (viii) the dsRNA comprises a duplex region of 12-40 nucleotide pairs in length; and (ix) a blunt end at 5′ end of the antisense strand.

In some embodiments, the invention provides a dsRNA agent that inhibits expression of a serine peptidase inhibitor, clade a, member 1 (Serpina1) gene, comprising a sense strand and an antisense strand, each strand having 14 to 40 nucleotides, wherein the antisense strand has sufficient complementarity to a target Serpina1 sequence, e.g., nucleotides 1440-1480 of SEQ ID NO:1, to mediate RNA interference and wherein the antisense strand comprises at least one thermally destabilizing modification of the duplex within the seed region (i.e., at position 2-9 of the 5′-end of the antisense strand, counting from the 5′-end), and the dsRNA further has at least one (e.g., one, two, three, four, five, six seven, eight or all nine) of the following characteristics: (i) a melting temperature (Tₘ) of from about 40° C. to about 80° C.; (ii) the antisense comprises 6, 7, 8, 9, 10, 11 or 12 2′-OMe modifications; (iii) the antisense comprises 1, 2, 3 or 4 phosphorothioate internucleotide linkages; (iv) the sense strand is conjugated with a ligand; (v) the sense strand comprises 6, 7, 8, 9, 10, 11 or 12 2′-OMe modifications; (vi) the sense strand comprises 1, 2, 3 or 4 phosphorothioate internucleotide linkages; (vii) the dsRNA comprises at least 1, 2, 3, 4 or 5 2′-deoxy modification(s); (viii) the dsRNA comprises a duplex region of 12-40 nucleotide pairs in length; and (ix) a blunt end at 5′ end of the antisense strand.

In some embodiments, the dsRNA has a melting temperature with a lower end of the range from about 40° C., 45° C., 50° C., 55° C., 60° C. or 65° C., and upper end of the arranger from about 70° C., 75° C. or 80° C. In some embodiments, the dsRNA has a melting temperature in the range from about 55° C. to about 70° C. In some embodiments, the dsRNA has a melting temperature in the range from about 57° C. to about 67° C. In some particular embodiments, the dsRNA has a melting temperature in the range from about 60° C. to about 67° C. In some additional embodiments, the dsRNA has a melting temperature in the range from about 62° C. to about 66° C.

It has also ben discovered that dsRNA agents having a melting temperature of at least 60° C. are more effective in vivo and in vitro. Thus, in some embodiments, the dsRNA has a melting temperature of at least 60° C.

In some embodiments, the dsRNA agent comprises a sense strand and an antisense strand, each strand having 14 to 40 nucleotides, wherein the antisense strand has sufficient complementarity to a target Serpina1 sequence, e.g., nucleotides 1440-1480 of SEQ ID NO:1, to mediate RNA interference and wherein the antisense strand comprises at least one thermally destabilizing modification of the duplex within the seed region (i.e., at position 2-9 of the 5′-end of the antisense strand, counting from the 5′-end), the dsRNA has a melting temperature (Tₘ) of from about 40° C. to about 80° C., and the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; (vii) the dsRNA comprises a duplex region of 12-40 nucleotide pairs in length; and (viii) a blunt end at 5′ end of the antisense strand.

In some embodiments, the dsRNA agent has a duplex region of 12-40 nucleotide pairs in length, wherein the antisense strand comprises at least one thermally destabilizing modification of the duplex within the seed region (i.e., at position 2-9 of the 5′-end of the antisense strand, counting from the 5′-end), and the dsRNA has a Tₘ of from about 40° C. to about 80° C., and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; and vii) a blunt end at 5′ end of the antisense strand.

In some embodiments, the dsRNA agent has a duplex region of 19, 21, 22 or 23 nucleotide base pairs in length, wherein the antisense strand contains at least one thermally destabilizing modification of the duplex located in the seed region of the antisense strand (i.e., at position 2-9 of the 5′-end of the antisense strand), and wherein the dsRNA has a melting temperature of about 40° C. to about 80° C.

In some embodiments, the dsRNA agent has a duplex region of 19, 21, 22 or 23 nucleotide base pairs in length, wherein the antisense strand contains at least one thermally destabilizing modification of the duplex located in the seed region of the antisense strand (i.e., at position 2-9 of the 5′-end of the antisense strand), and wherein the dsRNA has a melting temperature of about 40° C. to about 80° C. (e.g., 40° C., 50° C., 60° C., 70° C. or 80° C.).

In some particular embodiments, the thermally destabilizing modification of the duplex is at position 5, 6, 7, or 8 of the antisense strand, counting from 5′-end of the antisense strand.

In some particular embodiments, the thermally destabilizing modification of the duplex is at position 6 of the antisense strand, counting from 5′-end of the antisense strand.

In some particular embodiments, the thermally destabilizing modification of the duplex is at position 7 of the antisense strand, counting from 5′-end of the antisense strand.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference and wherein the antisense strand comprises at least one thermally destabilizing modification of the duplex within the seed region (i.e., at position 2-9 of the 5′-end of the antisense strand, counting from the 5′-end), wherein the dsRNA has a melting temperature of about 40° C. to about 80° C., and the antisense strand further comprises one or both of the following characteristics:
(i) 2, 3, 4, 5 or 6 2′-fluoro modifications; and
(ii) 1, 2, 3 or 4 phosphorothioate internucleotide linkages; and the sense strand comprises one, two or three of the following characteristics:
(i) a ligand conjugated with the sense strand;
(ii) 2, 3, 4 or 5 2′-fluoro modifications; and
(iii) 1, 2, 3 or 4 phosphorothioate internucleotide linkages.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference and wherein the antisense strand comprises at least one thermally destabilizing modification of the duplex within the first 9 nucleotide positions counting from the 5′-end, and a ligand is conjugated with the sense strand, and wherein the dsRNA has a melting temperature of about 40° C. to about 80° C.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference and wherein the antisense strand comprises at least one thermally destabilizing modification of the duplex within the first 9 nucleotide positions counting from the 5′-end, a ligand is conjugated with the sense strand, and the dsRNA comprises at least four 2′-fluoro modifications.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, said sense strand comprises a ligand, and wherein the dsRNA has a melting temperature of about 40° C. to about 80° C. In some further embodiments of this, the ligand is an ASGPR ligand.

In some embodiments, the dsRNA agent comprises a sense strand and an antisense strand, each strand having 14 to 40 nucleotides, wherein the antisense strand has sufficient complementarity to a Serina1 target sequence, e.g., nucleotides 1440-1480 of SEQ ID NO:1, to mediate RNA interference, wherein said antisense strand comprises at least one thermally destabilizing modification of the duplex located in position 4-8, counting from the 5′-end, wherein said sense strand comprises a ligand, wherein each of the sense and antisense strands comprise at least two 2′-fluoro modifications, and wherein the dsRNA has a melting temperature of about 40° C. to about 80° C. In some further embodiments of this, the ligand is an ASGPR ligand.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, wherein the dsRNA has a melting temperature of about 40° C. to about 80° C., and wherein the antisense further comprises at least two of the following characteristics: (i) the thermally destabilizing modification of the duplex is located in position 4 to 8 of the antisense strand; (ii) at least two 2′-fluoro modifications; (iii) phosphorothioate internucleotide linkages between nucleotide positions 1 and 2 (counting from the 5′ end); and antisense strand has a length of 18 to 35 nucleotides. In some further embodiments the ligand is an ASGPR ligand.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, wherein the dsRNA has a melting temperature of about 40° C. to about 80° C., and the sense strand has at least one of the following characteristics: (i) the ligand is attached to either end of the sense strand; (ii) sense strand comprises at least two 2′-fluoro modifications; and (iii) the sense strand and the antisense strand show sufficient complementarity to form a double stranded region spanning at least 19 nucleotide positions and wherein the thermally destabilizing modification of the duplex is located within said double stranded region.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, wherein the dsRNA has a melting temperature of about 40° C. to about 80° C., and wherein the thermally destabilizing modification of the duplex is selected from the group consisting of:

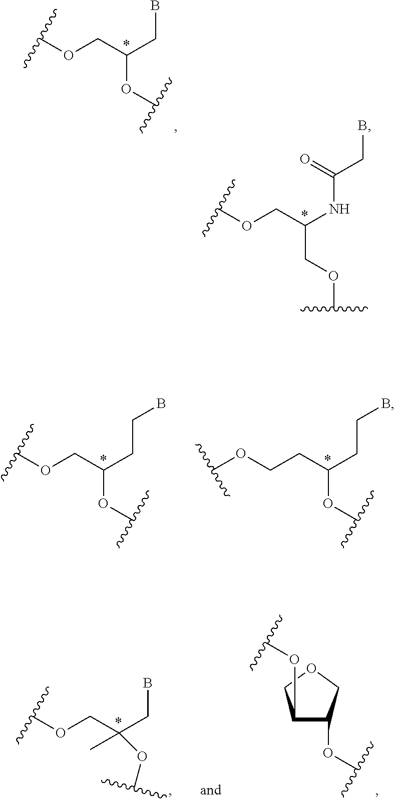

wherein B is a modified or unmodified nucleobase and the asteric on each stucture represents either R, S or racemic.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein said antisense strand comprises at least one thermally destabilizing modification of the duplex located in position 4-8, counting from the 5′-end, wherein said sense strand comprises a ligand, and wherein each of the sense and antisense strands comprise at least two 2′-fluoro modifications, wherein the dsRNA has a melting temperature of about 40° C. to about 80° C., and wherein the thermally destabilizing modification of the duplex is selected from the group consisting of:

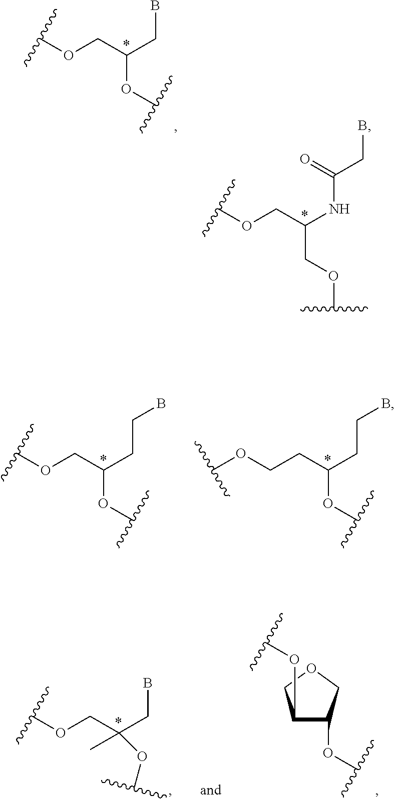

wherein B is a modified or unmodified nucleobase and the asteric on each stucture represents either R, S or racemic.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex located at position 7, counting from the 5′-end of the antisense strand, wherein said sense strand comprises a ligand, and wherein the dsRNA has a melting temperature of about 40° C. to about 80° C.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein said antisense strand comprises at least one thermally destabilizing modification of the duplex located at position 7, counting from the 5′-end, wherein said sense strand comprises a ligand, and wherein each of the sense and antisense strands comprise at least two 2′-fluoro modifications, wherein the dsRNA has a melting temperature of about 40° C. to about 80° C., and wherein the thermally destabilizing modification of the duplex is selected from the group consisting of:

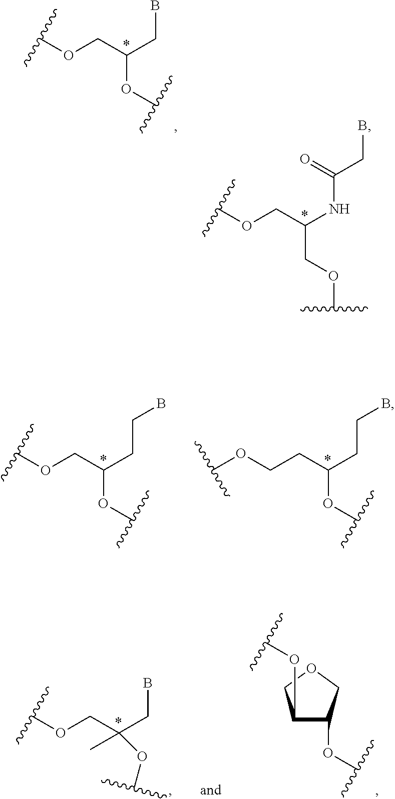

wherein B is a modified or unmodified nucleobase and the asteric on each stucture represents either R, S or racemic.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, wherein said sense strand comprises a ligand, wherein the dsRNA has a melting temperature of about 40° C. to about 80° C., and wherein the ligand comprises one or more GalNAc derivatives attached through a bivalent or trivalent branched linker.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, wherein the dsRNA has a melting temperature of about 40° C. to about 80° C., and wherein the ligand is an ASGPR ligand of structure:

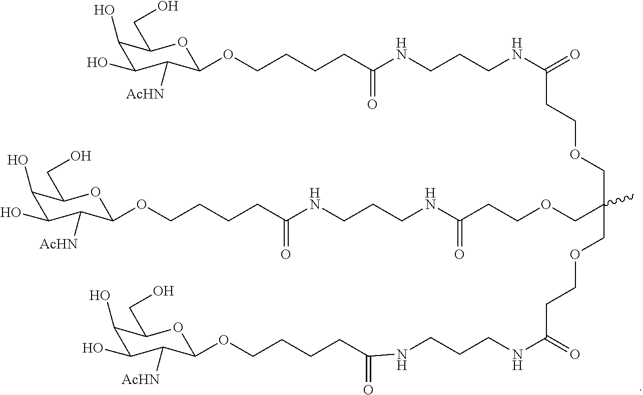 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 3 or 4 2′-fluoro modifications, and comprises 0, 1 or 2 phosphorothioate internucleotide linkages; wherein the antisense strand comprises 3, 4, 5 or 6 2′-fluoro modifications, comprises 2, 3 or 4 phosphorothioate internucleotide linkages; wherein the dsRNA has a melting temperature of about 40° C. to about 80° C.; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 2′-fluoro modifications at positions 7, 10 and 11 or at positions 7, 9, 10 and 11 (counting from 5′-end of the sense strand), and optionally comprises phosphorothioate internucleotide linkages between nucleotide positions 1 and 2, and between nucleotide positions 2 and 3; wherein the antisense strand comprises 3, 4, 5 or 6 2′-fluoro modifications, comprises 2, 3 or 4 phosphorothioate internucleotide linkages; wherein the dsRNA has a melting temperature of about 40° C. to about 80° C.; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 3 or 4 2′-fluoro modifications, and comprises 0 or 2 phosphorothioate internucleotide linkages; wherein the antisense strand comprises 2′-fluoro modifications at positions 2, 6, 8, 9, 14 or 16, or at positions 2, 6, 14 or 16, or at positions 2, 14 and 16; and the antisense comprises phosphorothioate internucleotide linkages between nucleotide positions 21 and 22, and between nucleotide positions 22 and 23; wherein the dsRNA has a melting temperature of about 40° C. to about 80° C.; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 3 or 4 2′-fluoro modifications, and comprises 0 or 2 phosphorothioate internucleotide linkages; wherein the antisense strand comprises 2′-fluoro modifications at positions 2, 6, 8, 9, 14 or 16, or at positions 2, 6, 14 or 16, or at positions 2, 14 and 16; and the antisense comprises phosphorothioate internucleotide linkages between nucleotide positions 21 and 22, between nucleotide positions 22 and 23, between nucleotide positions 1 and 2, between nucleotide positions 2 and 3; wherein the dsRNA has a melting temperature of about 40° C. to about 80° C.; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 2′-fluoro modifications at positions 7, 10 and 11 or at positions 7, 9, 10 and 11 (counting from 5′-end of the sense strand), and optionally comprises phosphorothioate internucleotide linkages between nucleotide positions 1 and 2, and between nucleotide positions 2 and 3; wherein the antisense strand comprises 2′-fluoro modifications at positions 2, 6, 8, 9, 14 or 16, or at positions 2, 6, 14 or 16, or at positions 2, 14 and 16; and the antisense comprises phosphorothioate internucleotide linkages between nucleotide positions 21 and 22, and between nucleotide positions 22 and 23; wherein the dsRNA has a melting temperature of about 40° C. to about 80° C.; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand. In some embodiments, the sense and antisense strands are independently 19, 20, 21, 22, 23, 24 or 25 nucleotides in length, wherein the antisense strand contains at least one thermally destabilizing nucleotide, and where the at least one thermally destabilizing nucleotide is in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; (vii) the dsRNA comprises a duplex region of 18, 19, 21, 22, 23, 24 or 24 nucleotide pairs in length; and (viii) the dsRNA comprises a blunt end at 5′-end of the sense strand. In some particular embodiments, sense strand is 19, 20 or 21 or 22 nucleotides in length and the antisense strand is 20, 21 or 22 nucleotides in length.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 2′-fluoro modifications at positions 7, 10 and 11 or at positions 7, 9, 10 and 11 (counting from 5′-end of the sense strand), and optionally comprises phosphorothioate internucleotide linkages between nucleotide positions 1 and 2, and between nucleotide positions 2 and 3; wherein the antisense strand comprises 2′-fluoro modifications at positions 2, 6, 8, 9, 14 or 16, or at positions 2, 6, 14 or 16, or at positions 2, 14 and 16; and the antisense comprises phosphorothioate internucleotide linkages between nucleotide positions 21 and 22, between nucleotide positions 22 and 23, between nucleotide positions 1 and 2, between nucleotide positions 2 and 3; wherein the dsRNA has a melting temperature of about 40° C. to about 80° C.; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, one end of the dsRNA is a blunt end and the other end has an overhang, wherein the antisense strand contains at least one thermally destabilizing nucleotide, and where the at least one thermally destabilizing nucleotide is in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; (vii) and the dsRNA comprises a duplex region of 12-40 nucleotide pairs in length. In some embodiments, the overhang is on the 3′-end of the antisense strand and the blunt end is at the 5′-end of the antisense strand. In some particular embodiments, the overhang is 2, 3 or 4-nucleotides in length.

In some embodiments, the dsRNA agent has a duplex region of 19, 21, 22 or 23 nucleotide base pairs in length, wherein one end of the dsRNA is a blunt end and the other end has an overhang, wherein the antisense strand contains at least one thermally destabilizing modification of the duplex located in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, five or all six) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications, and optionally the 2 nucleotide overhang is on the 3′-end of the antisense strand and the blunt end is at the 5′-end of the antisense strand. In some embodiments, the overhang is on the 3′-end of the antisense strand and the blunt end is at the 5′-end of the antisense strand.

In some embodiments, the dsRNA agent of the invention may also have two blunt ends, at both ends of the dsRNA duplex.

In some embodiments, the dsRNA has a blunt end at both ends of the duplex, wherein the antisense strand contains at least one thermally destabilizing nucleotide, and where the at least one thermally destabilizing nucleotide is in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; and (vii) the dsRNA comprises a duplex region of 12-40 nucleotide pairs in length.

In some embodiments, the dsRNA agent has a duplex region of 19, 21, 22 or 23 nucleotide base pairs in length and has a blunt end at both ends of the duplex, wherein one end of the dsRNA is a blunt end and the other end has an overhang, wherein the antisense strand contains at least one thermally destabilizing modification of the duplex located in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, five or all six) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications.

In some embodiments, the dsRNA agent of the invention comprises a 21 nucleotides (nt) sense strand and a 23 nucleotides (nt) antisense, wherein the antisense strand contains at least one thermally destabilizing nucleotide, where the at least one thermally destabilizing nucleotide occurs in the seed region of the antisense strand (i.e., at position 2-9 of the 5′-end of the antisense strand), wherein one end of the dsRNA is blunt, while the other end is comprises a 2 nt overhang, wherein the dsRNA has a melting temperature of about 40° C. to about 80° C., and wherein the dsRNA optinally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; and (vii) the dsRNA comprises a blunt end at 5′-end of the antisense strand. Preferably, the 2 nt overhang is at the 3′-end of the antisense.

In some embodiments, the dsRNA agent of the invention comprising a sense and antisense strands, wherein: the sense strand is 25-30 nucleotide residues in length, wherein starting from the 5′ terminal nucleotide (position 1), positions 1 to 23 of said sense strand comprise at least 8 ribonucleotides; antisense strand is 36-66 nucleotide residues in length and, starting from the 3′ terminal nucleotide, at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce Serpina1 target gene expression when said double stranded nucleic acid is introduced into a mammalian cell; and wherein the antisense strand contains at least one thermally destabilizing nucleotide, where at least one thermally destabilizing nucleotide is in the seed region of the antisense strand (i.e. at position 2-9 of the 5′-end of the antisense strand), and wherein the dsRNA has a melting temperature of about 40° C. to about 80° C. For example, the thermally destabilizing nucleotide occurs between positions opposite or complimentary to positions 14-17 of the 5′-end of the sense strand, and wherein the dsRNA optinally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications; and (vii) the dsRNA comprises a duplex region of 12-30 nucleotide pairs in length.

In some embodiments, the dsRNA agent of the invention comprises a sense and antisense strands, wherein said dsRNA agent comprises a sense strand having a length which is at least 25 and at most 29 nucleotides and an antisense strand having a length which is at most 30 nucleotides with the sense strand comprises a modified nucleotide that is susceptible to enzymatic degradation at position 11 from the 5′ end, wherein the 3′ end of said sense strand and the 5′ end of said antisense strand form a blunt end and said antisense strand is 1-4 nucleotides longer at its 3′ end than the sense strand, wherein the duplex region which is at least 25 nucleotides in length, and said antisense strand is sufficiently complementary to a Serina1 target mRNA along at least 19 nt of said antisense strand length to reduce target gene expression when said dsRNA agent is introduced into a mammalian cell, and wherein dicer cleavage of said dsRNA preferentially results in an siRNA comprising said 3′ end of said antisense strand, thereby reducing expression of the target gene in the mammal, wherein the antisense strand contains at least one thermally destabilizing nucleotide, where the at least one thermally destabilizing nucleotide is in the seed region of the antisense strand (i.e. at position 2-9 of the 5′-end of the antisense strand), wherein the dsRNA has a melting temperature of about 40° C. to about 80° C., and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications; and (vii) the dsRNA has a duplex region of 12-29 nucleotide pairs in length.

In some embodiments, the antisense strand comprises phosphorothioate internucleotide linkages between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 1, 2, 3 or 4 phosphorothioate internucleotide linkages; (v) the dsRNA comprises at least four 2′-fluoro modifications; (vi) the dsRNA comprises a duplex region of 12-40 nucleotide pairs in length; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, wherein the antisense strand contains at least one thermally destabilizing modification of the duplex located in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 3 or 4 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), wherein the dsRNA has a melting temperature of about 40° C. to about 80° C., and wherein the dsRNA optionally further has at least one (e.g., one, two, three, four, five, six or all seven) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 3 or 4 phosphorothioate internucleotide linkages; (v) the dsRNA comprises at least four 2′-fluoro modifications; (vi) the dsRNA comprises a duplex region of 12-40 nucleotide pairs in length; and (vii) the dsRNA has a blunt end at 5′-end of the antisense strand.

In one aspect the invention provides a dsRNA agent capable of inhibiting the expression of a Serpina1 target sequence, e.g., nucleotides 1440-1480 of SEQ ID NO:1, comprising a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference and wherein the antisense strand comprises at least one thermally destabilizing modification of the duplex within the seed region (i.e., at position 2-9 of the 5′-end of the antisense strand, counting from the 5′-end), and the dsRNA further has at least one (e.g., one, two, three, four, five, six seven or all eight) of the following characteristics:
(i) the antisense comprises 2, 3, 4, 5 or 6 2′-fluoro modifications;
(ii) the antisense comprises 1, 2, 3 or 4 phosphorothioate internucleotide linkages;
(iii) the sense strand is conjugated with a ligand;
(iv) the sense strand comprises 2, 3, 4 or 5 2′-fluoro modifications;
(v) the sense strand comprises 1, 2, 3 or 4 phosphorothioate internucleotide linkages;
(vi) the dsRNA comprises at least four 2′-fluoro modifications;
(vii) the dsRNA comprises a duplex region of 12-40 nucleotide pairs in length; and
(viii) a blunt end at 5′ end of the antisense strand.

In some particular embodiments, the thermally destabilizing modification of the duplex is at position 7 of the antisense strand, counting from 5′-end of the antisense strand.

In some embodiments, the dsRNA agent comprises a sense strand and an antisense strand, each strand having 14 to 40 nucleotides, wherein the antisense strand has sufficient complementarity to the target sequence to mediate RNA interference and wherein the antisense strand comprises at least one thermally destabilizing modification of the duplex within the seed region (i.e., at position 2-9 of the 5′-end of the antisense strand, counting from the 5′-end), and the antisense strand further comprises one or both of the following characteristics:
(iii) 2, 3, 4, 5 or 6 2′-fluoro modifications; and
(iv) 1, 2, 3 or 4 phosphorothioate internucleotide linkages; and the sense strand comprises one, two or three of the following characteristics:
(iv) a ligand conjugated with the sense strand;
(v) 2, 3, 4 or 5 2′-fluoro modifications; and
(vi) 1, 2, 3 or 4 phosphorothioate internucleotide linkages.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference and wherein the antisense strand comprises at least one thermally destabilizing modification of the duplex within the first 9 nucleotide positions counting from the 5′-end, and a ligand is conjugated with the sense strand.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference and wherein the antisense strand comprises at least one thermally destabilizing modification of the duplex within the first 9 nucleotide positions counting from the 5′-end, a ligand is conjugated with the sense strand, and the dsRNA comprises at least four 2′-fluoro modifications.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand. In some further embodiments of this, the ligand is an ASGPR ligand.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference, wherein said antisense strand comprises at least one thermally destabilizing modification of the duplex located in position 4-8, counting from the 5′-end, wherein said sense strand comprises a ligand, and wherein each of the sense and antisense strands comprise at least two 2′-fluoro modifications. In some further embodiments of this, the ligand is an ASGPR ligand.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, and wherein the antisense further comprises at least two of the following characteristics: (i) the thermally destabilizing modification of the duplex is located in position 4 to 8 of the antisense strand; (ii) at least two 2′-fluoro modifications; (iii) phosphorothioate internucleotide linkages between nucleotide positions 1 and 2 (counting from the 5′ end); and antisense strand has a length of 18 to 35 nucleotides. In some further embodiments the ligand is an ASGPR ligand.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, and the sense strand has at least one of the following characteristics: (i) the ligand is attached to either end of the sense strand; (ii) sense strand comprises at least two 2′-fluoro modifications; and (iii) the sense strand and the antisense strand show sufficient complementarity to form a double stranded region spanning at least 19 nucleotide positions and wherein the thermally destabilizing modification of the duplex is located within said double stranded region.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, and wherein the thermally destabilizing modification of the duplex is selected from the group consisting of:

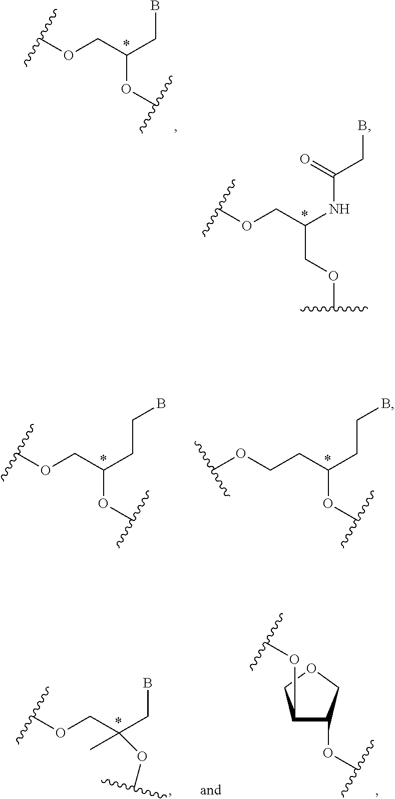

wherein B is a modified or unmodified nucleobase and the asteric on each stucture represents either R, S or racemic.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference, wherein said antisense strand comprises at least one thermally destabilizing modification of the duplex located in position 4-8, counting from the 5′-end, wherein said sense strand comprises a ligand, and wherein each of the sense and antisense strands comprise at least two 2′-fluoro modifications, and wherein the thermally destabilizing modification of the duplex is selected from the group consisting of:

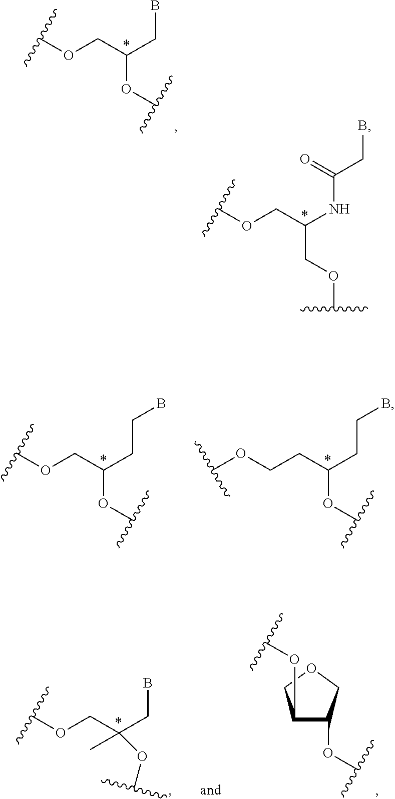

wherein B is a modified or unmodified nucleobase and the asteric on each stucture represents either R, S or racemic.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex located at position 7, counting from the 5′-end of the antisense strand, and wherein said sense strand comprises a ligand.

In some embodiments, the dsRNA agent comprises a sense strand and an antisense strand, each strand having 14 to 40 nucleotides, wherein the antisense strand has sufficient complementarity to the Serpina1 target sequence, e.g., nucleotides 1440-1480 of SEQ ID NO:1, to mediate RNA interference, wherein said antisense strand comprises at least one thermally destabilizing modification of the duplex located at position 7, counting from the 5′-end, wherein said sense strand comprises a ligand, and wherein each of the sense and antisense strands comprise at least two 2′-fluoro modifications, and wherein the thermally destabilizing modification of the duplex is selected from the group consisting of:

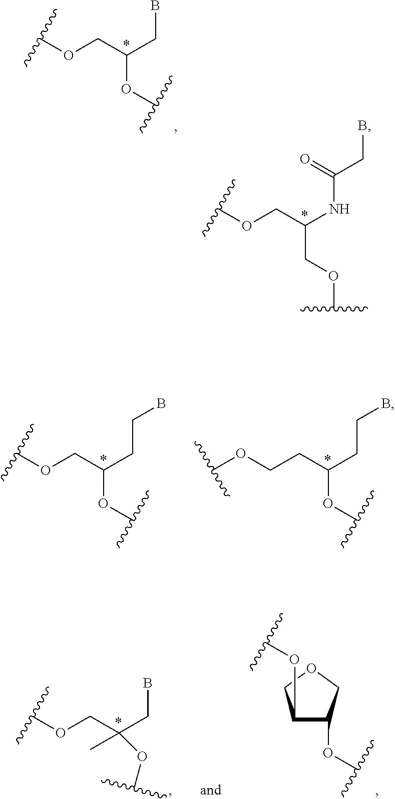

wherein B is a modified or unmodified nucleobase and the asteric on each stucture represents either R, S or racemic.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, wherein the ligand comprsies one or more GalNAc derivatives attached through a bivalent or trivalent branched linker.

In some embodiments, the dsRNA agent comprises a sense strand and an antisense strand, each strand having 14 to 40 nucleotides, wherein the antisense strand has sufficient complementarity to a Serpina1 target sequence, e.g., nucleotides 1440-1480 of SEQ ID NO:1, to mediate RNA interference, wherein the dsRNA comprises at least four 2′-fluoro, wherein said antisense strand comprises at least one thermally destabilizing modification of the duplex within the first 9 nucleotide positions, counting from the 5′-end, and wherein said sense strand comprises a ligand, wherein the ligand is an ASGPR ligand of structure:

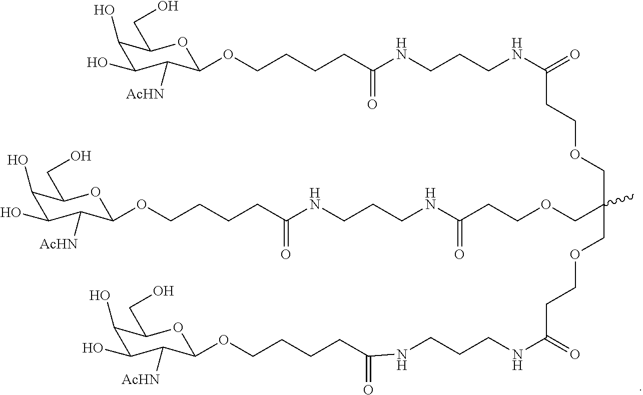 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 3 or 4 2′-fluoro modifications, and comprises 0, 1 or 2 phosphorothioate internucleotide linkages; wherein the antisense strand comprises 3, 4, 5 or 6 2′-fluoro modifications, comprises 2, 3 or 4 phosphorothioate internucleotide linkages; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 2′-fluoro modifications at positions 7, 10 and 11 or at positions 7, 9, 10 and 11 (counting from 5′-end of the sense strand), and optionally comprises phosphorothioate internucleotide linkages between nucleotide positions 1 and 2, and between nucleotide positions 2 and 3; wherein the antisense strand comprises 3, 4, 5 or 6 2′-fluoro modifications, comprises 2, 3 or 4 phosphorothioate internucleotide linkages; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 3 or 4 2′-fluoro modifications, and comprises 0 or 2 phosphorothioate internucleotide linkages; wherein the antisense strand comprises 2′-fluoro modifications at positions 2, 6, 8, 9, 14 or 16, or at positions 2, 6, 14 or 16, or at positions 2, 14 and 16; and the antisense comprises phosphorothioate internucleotide linkages between nucleotide positions 21 and 22, and between nucleotide positions 22 and 23; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 3 or 4 2′-fluoro modifications, and comprises 0 or 2 phosphorothioate internucleotide linkages; wherein the antisense strand comprises 2′-fluoro modifications at positions 2, 6, 8, 9, 14 or 16, or at positions 2, 6, 14 or 16, or at positions 2, 14 and 16; and the antisense comprises phosphorothioate internucleotide linkages between nucleotide positions 21 and 22, between nucleotide positions 22 and 23, between nucleotide positions 1 and 2, between nucleotide positions 2 and 3; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 2′-fluoro modifications at positions 7, 10 and 11 or at positions 7, 9, 10 and 11 (counting from 5′-end of the sense strand), and optionally comprises phosphorothioate internucleotide linkages between nucleotide positions 1 and 2, and between nucleotide positions 2 and 3; wherein the antisense strand comprises 2′-fluoro modifications at positions 2, 6, 8, 9, 14 or 16, or at positions 2, 6, 14 or 16, or at positions 2, 14 and 16; and the antisense comprises phosphorothioate internucleotide linkages between nucleotide positions 21 and 22, and between nucleotide positions 22 and 23; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In some embodiments, the sense and antisense strands are independently 19, 20, 21, 22, 23, 24 or 25 nucleotides in length; the antisense strand contains at least one thermally destabilizing nucleotide, where the at least one thermally destabilizing nucleotide is in the seed region of the antisense strand (i.e., at position 2-9 of the 5′-end of the antisense strand); wherein the sense strand is conjugated with a ligand, comprises 2′-fluoro modifications at positions 7, 10 and 11 or at positions 7, 9, 10 and 11 (counting from 5′-end of the sense strand), and optionally comprises phosphorothioate internucleotide linkages between nucleotide positions 1 and 2, and between nucleotide positions 2 and 3; wherein the antisense strand comprises 2′-fluoro modifications at positions 2, 6, 8, 9, 14 or 16, or at positions 2, 6, 14 or 16, or at positions 2, 14 and 16; and the antisense comprises phosphorothioate internucleotide linkages between nucleotide positions 21 and 22, between nucleotide positions 22 and 23, between nucleotide positions 1 and 2, between nucleotide positions 2 and 3; and wherein the dsRNA optionally further has at least one (e.g., one, two or all three) of the following characteristics: (i) the dsRNA comprises a duplex region of 12-25 nucleotide pairs in length; (ii) the dsRNA comprises a blunt end at 5′-end of the antisense strand; and (iii) the dsRNA has at least a two nucleotide overhang at the 3′-end of the antisense strand.

In a particular embodiment, the dsRNA agents of the present invention comprise:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker; and
   (iii) 2′-F modifications at positions 7, 10, and 11 (counting from the 5′ end); and
(b) an antisense strand having:
   (i) a length of 23 nucleotides;
   (ii) 2′-F modifications at positions 2, 6 to 8, 9, 14, and 16 (counting from the 5′ end);
   (iii) phosphorothioate internucleotide linkages between nucleotide positions 21 and 22, and between nucleotide positions 22 and 23 (counting from the 5′ end); and
   (iv) a thermally destabilizing modification of the duplex at position 7 (counting from the 5′ end);
      wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 7, 9, 10, and 11 (counting from the 5′ end); and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-F modifications at positions 2, 6, 14, and 16 (counting from the 5′ end);
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end); and
   (iv) a thermally destabilizing modification of the duplex at position 7 (counting from the 5′ end);
      wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 7, 9, 10, and 11 (counting from the 5′ end); and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-F modifications at positions 2, 14, and 16 (counting from the 5′ end);
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end); and
   (iv) a thermally destabilizing modification of the duplex at position 6 or 7 (counting from the 5′ end);
      wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 7, 9, 10, and 11 (counting from the 5′ end); and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-F modifications at positions 2, 6, 8, 9, 14, and 16 (counting from the 5′ end);
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end); and
   (iv) a thermally destabilizing modification of the duplex at position 7 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 7, 9, 10, and 11 (counting from the 5′ end); and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-F modifications at positions 2, 14, and 16 (counting from the 5′ end);
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end); and
   (iv) a thermally destabilizing modification of the duplex at position 7 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprising an antisense strand having:
(i) 2′-F modifications at positions 2, 14, and 16 (counting from the 5′ end); and
(2) a thermally destabilizing modification of the duplex at position 6 or 7 (counting from the 5′ end).

In another particular embodiment, the dsRNA agents of the present invention comprise:
(a) a sense strand having:
   (i) an ASGPR ligand, wherein said ASGPR ligand;
   (ii) phosphorothioate internucleotide linkages between nucleotide positions 1 and 2, and between nucleotide positions 2 and 3 (counting from the 5′ end);
   and
(b) an antisense strand having:
   (i) 2′-F modifications at positions 2, 14, and 16 (counting from the 5′ end);
   (ii) a thermally destabilizing modification of the duplex at position 6 or 7 (counting from the 5′ end);

In another particular embodiment, the dsRNA agents of the present invention comprise:
(a) a sense strand having:
   (i) an ASGPR ligand attached to the 3′-end, wherein said ASGPR ligand comprises three GalNAc derivatives attached through a trivalent branched linker;
   (ii) phosphorothioate internucleotide linkages between nucleotide positions 1 and 2, and between nucleotide positions 2 and 3 (counting from the 5′ end);
and
(b) an antisense strand having:
   (ii) 2′-F modifications at positions 2, 14, and 16 (counting from the 5′ end); (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end); and
   (iv) a thermally destabilizing modification of the duplex at position 6 or 7 (counting from the 5′ end);
   wherein the dsRNA agents have a two nucleotide overhang at the 3′-end of the antisense strand, and a blunt end at the 5′-end of the antisense strand.

In some embodiments, the dsRNA agent further comprises at least one ASGPR ligand. For example, the ASGPR ligand is one or more GalNAc derivatives attached through a bivalent or trivalent branched linker, such as:

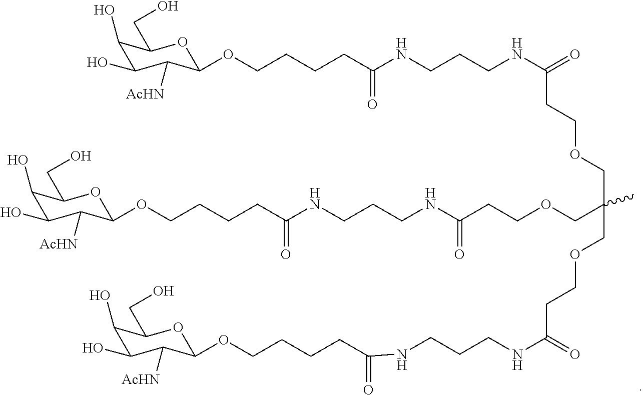 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one example, the ASGPR ligand is attached to the 3′ end of the sense strand.

The region of a Serpina1 mRNA targeted by any of the dsRNA agents described herein may be nucleotides 1440-1480 of SEQ ID NO:1; or nucleotides 1441-1479 of SEQ ID NO:1; or nucleotides 1442-1478 of SEQ ID NO:1; or nucleotides 1443-1477 of SEQ ID NO:1; or nucleotides 1444-1476 of SEQ ID NO:1; or nucleotides 1445-1475 of SEQ ID NO:1; or nucleotides 1446-1474 of SEQ ID NO:1; or nucleotides 1447-1473 of SEQ ID NO:1; or nucleotides 1448-1473 of SEQ ID NO:1; or nucleotides 1448-1472 of SEQ ID NO:1; or nucleotides 1448-1471 of SEQ ID NO:1; or nucleotides 1448-1470 of SEQ ID NO:1; or nucleotides 1447-1469 of SEQ ID NO:1; or nucleotides 1446-1478 of SEQ ID NO:1; or nucleotides 1449-1471 of SEQ ID NO:1; or nucleotides 1450-1472 of SEQ ID NO:1; or nucleotides 1440-1475 of SEQ ID NO:1; or nucleotides 1445-1480 of SEQ ID NO:1; or nucleotides 1445-1475 of SEQ ID NO:1.

The present invention also provides vetors, cells, and pharmaceutical compositions comprising the dsRNA agents of the inevtion.

The pharmaceutical compositions of the invention may be an unbuffered solution, e.g., saline or water; or a buffered solution, e.g., a buffered solution comprising acetate, citrate, prolamine, carbonate, or phosphate or any combination thereof, or phosphate buffered saline (PBS).

In one aspect, the present invention provides a method of inhibiting Serpina1 expression in a cell. The method includes contacting the cell with any of the foregoing dsRNA agents or pharmaceutical composition of the invention, thereby inhibiting expression of the Serpina1 gene in the cell.

In one embodiment, the cell is in a subject, such as a human subject.

In one embodiment, the Serpina1 expression is inhibited by at least about 30%, about 40%, about 50%, about 60%, about 70%, about 80%, about 90%, about 95%, about 98%, or about 100%.

In one aspect, the present invention provides a method of treating a subject having a Serpina1 associated disease. The method includes administering to the subject a therapeutically effective amount of any of the foregoing dsRNA agents or pharmaceutical composition of the invention, thereby treating said subject.

In one embodiment, the subject is a human.

In one embodiment, the Serpina1 associated disease is a liver disorder.

In one embodiment, the liver disorder is selected from the group consisting of chronic liver disease, liver inflammation, cirrhosis, liver fibrosis, and/or hepatocellular carcinoma.

In another aspect, the present invention provides a method of inhibiting development of hepatocellular carcinoma in a subject having a Serpina1 deficiency variant. The method includes administering to the subject a therapeutically effective amount of any of the foregoing dsRNA agents or pharmaceutical composition of the invention, thereby inhibiting development of hepatocellular carcinoma in the subject.

In yet another aspect, the present invention provides a method of reducing the accumulation of misfolded Serpina1 in the liver of a subject having a Serpina1 deficiency variant. The method includes administering to the subject a therapeutically effective amount of any of the foregoing dsRNA agents or pharmaceutical composition of the invention, thereby reducing the accumulation of misfolded Serpina1 in the liver of the subject.

The dsRNA agent may administered to the subject at a dose of about 0.01 mg/kg to about 10 mg/kg or about 0.5 mg/kg to about 50 mg/kg; or a dose of about 10 mg/kg to about 30 mg/kg.

The dsRNA agent may administered to the subject subcutaneously; or intravenously.

The dsRNA agent may administered to the subject in two or more doses.

The present invention is further illustrated by the following detailed description and drawings.

### BRIEF DESCRIPTION OF THE DRAWINGS

FIG. 1A schematically depicts the modified sense and antisense strand nucleotide sequences of AD-61444; AD-75994; and AD-75995. FIG. 1A discloses SEQ ID NOs: 36-37, 36, 38, 36 and 39, respectively, in order of appearance.

FIG. 1B is a graph depicting the in vivo effect of administration of the indicated dsRNA agents on the level of expression of AAT (Serpina). The level of expression of AAT shown is relative to the pre-bleed level of expression of AAT.

FIG. 1C schematically depicts the modified sense and antisenes strand nucleotide sequences of AD-61444; AD-77404; and AD-77412. FIG. 1C discloses SEQ ID NOs: 36-37, 36-37, 36 and 39, respectively, in order of appearance.

FIG. 1D is a graph depicting the in vivo effect of administration of the indicated dsRNA agents on the level of expression of AAT (Serpina).

FIG. 2A is a graph depicting the off-target effect of AD-61444 in Hep3B cells tranfected with 10 nM of the dsRNA agent 16 hours after treatment.

FIG. 2B is a graph depicting the off-target effect of AD-77412 in Hep3B cells tranfected with 10 nM of the dsRNA agent 16 hours after treatment.

FIG. 3A is a graph depicting the efficacy and durability of AAT silencing in nonhuman primates (NHP) administered a single 0.3 mg/kg dose of the indicated agents. The level of expression of AAT shown is relative to the pre-bleed level of expression of AAT determined at days −7 and −1 pre-dose.

FIG. 3B is a graph depicting the durability of AAT silencing in nonhuman primates administered a single 1 mg/kg dose of the indicated agents. The level of expression of AAT shown is relative to the pre-bleed level of expression of AAT determined at days −7 and −1 pre-dose.

FIG. 3C is a graph depicting the durability of AAT silencing in nonhuman primates administered a single 3 mg/kg dose of the indicated agents. The level of expression of AAT shown is relative to the pre-bleed level of expression of AAT determined at days −7 and −1 pre-dose.

FIG. 3D is a graph depicting the durability of AAT silencing in nonhuman primates administered a single 10 mg/kg dose of the indicated agents. The level of expression of AAT shown is relative to the pre-bleed level of expression of AAT determined at days −7 and −1 pre-dose.

### DETAILED DESCRIPTION OF THE INVENTION

The present invention provides compositions comprising agents, e.g., RNAi agents, e.g., double stranded iRNA agents, targeting Serpina1. Also disclosed are methods using the compositions of the invention for inhibiting Serpina1 expression and for treating Serpina1 associated diseases, such as liver disorders, e.g., chronic liver disease, liver inflammation, cirrhosis, liver fibrosis, and/or hepatocellular carcinoma.

The present invention is based, at least in part on the discovery of effective nucleotide or chemical motifs for dsRNA agents targeting Serpina1 which are advantageous for inhibition of target gene expression, while having reduced off-target gene silencing effects, as well as compositions comprising such agents suitable for therapeutic use. More specifically, it has been discovered inter alia that dsRNA agents targeting Serpina1 where the antisense strand comprises at least one thermally destabilizing modification of the duplex within the seed region (i.e., at position 2-9 of the 5′-end of the antisense strand, counting from the 5′-end) and/or the dsRNA agent has a melting temperature in the range of from about 40° C. to about 80° C. can be more effective in mediating RNA interference than a parent dsRNA agent lacking the destabilizing modification.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, “Serpina1” refers to the serpin peptidase inhibitor, clade A, member 1 gene or protein. Serpina1 is also known as alpha-1-antitrypsin, α-1-antitrypsin, AAT, protease inhibitor 1, PI, PI1, anti-elastase, and antitrypsin.

The term Serpina1 includes human Serpina1, the amino acid and nucleotide sequence of which may be found in, for example, GenBank Accession Nos. GI:189163524 (SEQ ID NO:1), GI:189163525 (SEQ ID NO:2), GI:189163526 (SEQ ID NO:3), GI:189163527 (SEQ ID NO:4), GI:189163529 (SEQ ID NO:5), GI:189163531 (SEQ ID NO:6), GI:189163533 (SEQ ID NO:7), GI:189163535 (SEQ ID NO:8), GI:189163537 (SEQ ID NO:9), GI:189163539 (SEQ ID NO:10), and/or GI:189163541 (SEQ ID NO:11); rhesus Serpina1, the amino acid and nucleotide sequence of which may be found in, for example, GenBank Accession Nos. GI:402766667 (SEQ ID NO:12), GI:297298519 (SEQ ID NO:13), and/or GI: 297298520 (SEQ ID NO:14); mouse Serpina1, the amino acid and nucleotide sequence of which may be found in, for example, GenBank Accession No. GI:357588423 and/or GI:357588426; and rat, the amino acid and nucleotide sequence of which may be found in, for example, GenBank Accession No. GI:77020249. Additional examples of Serpina1 mRNA sequences are readily available using, e.g., GenBank and OMIM.

Over 120 alleles of Serpina1 have been identified and the “M” alleles are considered the wild-type or “normal” allele (e.g., “PIM1-ALA213” (also known as PI, M1A), “PIM1-VAL213” (also known as PI, MIV), “PIM2”, “PIM3”, and PIM4”). Additional variants may be found in, for example, the A(1)ATVar database (see, e.g., Zaimidou, S., et al. (2009) Hum Mutat. 230(3):308-13 and www.goldenhelix.org/A1ATVar).

As used herein, the term “Serpina1 deficiency allele” refers to a variant allele that produces proteins which do not fold properly and may aggregate intracellularly and are, thus, not properly transported from the site of synthesis in the liver to the site of action within the body.

Exemplary Serpina1 deficiency alleles include, the “Z allele”, the “S allele”, the “PIM(Malton) allele”, and the “PIM(Procida) allele”.

As used herein, the terms “Z allele”, “PIZ” and “Z-AAT” refer to a variant allele of Serpina1 in which the amino acid at position 342 of the protein is changed from a glutamine to a lysine as a result of the relevant codon being changed from GAG to AAG. A subject homozygous for a Z allele can be referred to as “PIZZ.” Z-AAT mutations account for 95% of Serpina1 deficiency patients and are estimated to be present in 100,000 Americans and about 3 million individuals worldwide. The Z allele reaches polymorphic frequencies in Caucasians and is rare or absent in Asians and blacks. The homozygous ZZ phenotype is associated with a high risk of both emphysema and liver disease. Z-AAT protein does not fold correctly in the endoplasmic reticulum, leading to loop-sheet polymers which aggregate and reduce secretion, elicitation of the unfolded protein response, apoptosis, endoplasmic reticulum overload response, autophagy, mitochondrial stress, and altered hepatocyte function.

As used herein, the terms “PIM(Malton)” and “M(Malton)-AAT” refer to a variant allele of Serpina1 in which one of the adjacent phenylalanine residues at position 51 or 52 of the mature protein is deleted. Deletion of this one amino acid shortens one strand of the beta-sheet, B6, preventing normal processing and secretion in the liver which is associated with hepatocyte inclusions and impaired secretion of the protein from the liver.

As used herein, the term “PIS” refers to a variant allele of Serpina1 in which a glutamic acid at position 264 is substituted with valine. Although the majority of this variant protein is degraded intracellularly, there is a high frequency of the PIS allele in the Caucasian population and, thus, compound heterozygotes with a Z or null allele are frequent.

As used herein, “target sequence” refers to a contiguous portion of the nucleotide sequence of an mRNA molecule formed during the transcription of a Serpina1 gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

“G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine, 2′-deoxythymidine or thymidine. However, it will be understood that the term “ribonucleotide” or “nucleotide” or “deoxyribonucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

“Polynucleotides,” also referred to as “oligonucleotides,” are formed through the covalent linkage of adjacent nucleosides to one another, to form a linear polymeric oligonucleotide. Within the polynucleotide structure, the phosphate groups are commonly referred to as forming the internucleoside linkages of the polynucleotide. Polynucleotides may be either RNA or DNA and are, for example less than about 100, 200, 300, or about 400 nucleotides in length.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of Serpina1 in a cell, e.g., a cell within a subject, such as a mammalian subject.

As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an siRNA agent of 21 to 23 nucleotides.

In one embodiment, an RNAi agent of the invention includes a single stranded RNA that interacts with a target RNA sequence, e.g., a Serpina1 target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (sssiRNA) generated within a cell and which promotes the formation of a RISC complex to effect silencing of the target gene, i.e., a Serpina1 gene. Accordingly, the term “siRNA” is also used herein to refer to an RNAi as described above.

In another embodiment, the RNAi agent may be a single-stranded RNAi agent that is introduced into a cell or organism to inhibit a target mRNA. Single-stranded RNAi agents (ssRNAi) bind to the RISC endonuclease, Argonaute 2, which then cleaves the target mRNA. The single-stranded siRNAs are generally 15-30 nucleotides and are chemically modified. The design and testing of single-stranded RNAi agents are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150; 883-894.

In another embodiment, an “iRNA” for use in the compositions and methods of the invention is a double-stranded RNA and is referred to herein as a “double stranded RNAi agent,” “double-stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., an Serpina1 gene. In some embodiments of the invention, a double-stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA agent are ribonucleotides, but as described in detail herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. As used herein, the term “modified nucleotide” refers to a nucleotide having, independently, a modified sugar moiety, a modified internucleotide linkage, and/or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “RNAi agent” for the purposes of this specification and claims.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, at least 3, at least 4, at least 5, at least 6, at least 7, at least 8, at least 9, at least 10, at least 20, at least 23 or more unpaired nucleotides.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one embodiment, an RNAi agent of the invention is a dsRNA, each strand of which comprises 19-23 nucleotides, that interacts with a target RNA sequence, e.g., a Serpina1 target mRNA sequence, to direct the cleavage of the target RNA. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188).

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end and/or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end and/or the 5′-end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 10-30 nucleotides, 10-25 nucleotides, 10-20 nucleotides or 10-15 nucleotides in length. In certain embodiments, an extended overhang is on the sense strand of the duplex. In certain embodiments, an extended overhang is present on the 3′ end of the sense strand of the duplex. In certain embodiments, an extended overhang is present on the 5′ end of the sense strand of the duplex. In certain embodiments, an extended overhang is on the antisense strand of the duplex. In certain embodiments, an extended overhang is present on the 3′ end of the antisense strand of the duplex. In certain embodiments, an extended overhang is present on the 5′ end of the antisense strand of the duplex. In certain embodiments, one or more of the nucleotides in the extended overhang is replaced with a nucleoside thiophosphate.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

The term “antisense strand” or “guide strand” refers to the strand of an iRNA, e.g., a dsRNA, which includes a region that is substantially complementary to a target sequence, e.g., a Serpina1 mRNA.

As used herein, the term “region of complementarity” refers to the region on the antisense strand that is substantially complementary to a sequence, for example a target sequence, e.g., a Serpina1 nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the iRNA.

The term “sense strand” or “passenger strand” as used herein, refers to the strand of an iRNA that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. For example, a complementary sequence is sufficient to allow the relevant function of the nucleic acid to proceed, e.g., RNAi. The skilled person will be able to determine the set of conditions most appropriate for a test of complementarity of two sequences in accordance with the ultimate application of the hybridized nucleotides.

Sequences can be “fully complementary” with respect to each when there is base-pairing of the nucleotides of the first nucleotide sequence with the nucleotides of the second nucleotide sequence over the entire length of the first and second nucleotide sequences. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary,” “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding Serpina1) including a 5′ UTR, an open reading frame (ORF), or a 3′ UTR. For example, a polynucleotide is complementary to at least a part of a Serpina1 mRNA if the sequence is substantially complementary to a non-interrupted portion of an mRNA encoding Serpina1.

In some embodiments, a dsRNA agent of the invention is “sufficiently complementary” to a Serpina1 target RNA, e.g., a target mRNA, such that the dsRNA agent silences production of protein encoded by the target mRNA. In another embodiment, the dsRNA agent of the invention is “exactly complementary” or fully complementary to a target RNA, e.g., the target RNA and the dsRNA duplex agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the dsRNA agent of the invention specifically discriminates a single-nucleotide difference. In this case, the dsRNA agent only mediates RNAi if exact complementary is found in the region (e.g., within 7 nucleotides of) the single-nucleotide difference.

The term ‘BNA’ refers to bridged nucleic acid, and is often referred as constrained or inaccessible RNA. BNA can contain a 5-, 6-membered, or even a 7-membered bridged structure with a “fixed” C₃′-endo sugar puckering. The bridge is typically incorporated at the 2′-, 4′-position of the ribose to afford a 2′,4′-BNA nucleotide (e.g., LNA, or ENA). Examples of BNA nucleotides include the following nucleosides:

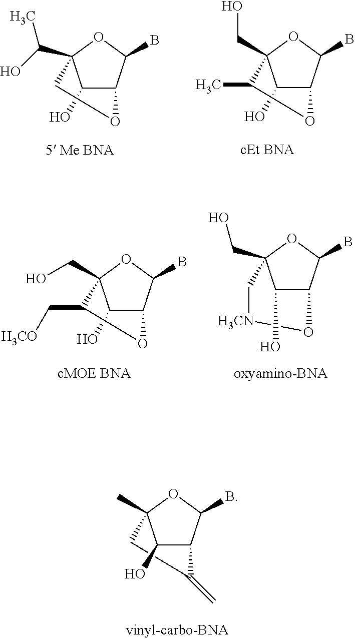

The term ‘LNA’ refers to locked nucleic acid, and is often referred as constrained or inaccessible RNA. LNA is a modified RNA nucleotide. The ribose moiety of an LNA nucleotide is modified with an extra bridge (e.g., a methylene bridge or an ethylene bridge) connecting the 2′ hydroxyl to the 4′ carbon of the same ribose sugar. For instance, the bridge can “lock” the ribose in the 3′-endo North) conformation:

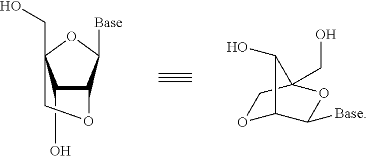

The term ‘ENA’ refers to ethylene-bridged nucleic acid, and is often referred as constrained or inaccessible RNA.

The term “inhibiting,” as used herein, is used interchangeably with “reducing,” “silencing,” “downregulating,” “suppressing” and other similar terms, and includes any level of inhibition.

The phrase “inhibiting expression of a Serpina1,” as used herein, includes inhibition of expression of any Serpina1 gene (such as, e.g., a mouse Serpina1 gene, a rat Serpina1 gene, a monkey Serpina1 gene, or a human Serpina1 gene) as well as variants, (e.g., naturally occurring variants), or mutants of a Serpina1 gene. Thus, the Serpina1 gene may be a wild-type Serpina1 gene, a variant Serpina1 gene, a mutant Serpina1 gene, or a transgenic Serpina1 gene in the context of a genetically manipulated cell, group of cells, or organism.

“Inhibiting expression of a Serpina1 gene” includes any level of inhibition of a Serpina1 gene, e.g., at least partial suppression of the expression of a Serpina1 gene, such as an inhibition of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

The expression of a Serpina1 gene may be assessed based on the level of any variable associated with Serpina1 gene expression, e.g., Serpina1 mRNA level, Serpina1 protein level, or serum AAT levels. Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

The phrase “contacting a cell with a double stranded RNAi agent,” as used herein, includes contacting a cell by any possible means. Contacting a cell with a double stranded RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, the bloodstream or the subcutaneous space, such that the agent will subsequently reach the tissue where the cell to be contacted is located. For example, the RNAi agent may contain and/or be coupled to a ligand, e.g., a GalNAc3 ligand, that directs the RNAi agent to a site of interest, e.g., the liver. Combinations of in vitro and in vivo methods of contacting are also possible. In connection with the methods of the invention, a cell might also be contacted in vitro with an RNAi agent and subsequently transplanted into a subject.

A “patient” or “subject,” as used herein, is intended to include either a human or non-human animal, preferably a mammal, e.g., a monkey. Most preferably, the subject or patient is a human.

A “Serpina1 associated disease,” as used herein, is intended to include any disease, disorder, or condition associated with the Serpina1 gene or protein. Such a disease may be caused, for example, by misfolding of a Serpina1 protein, intracellular accumulation of Serpina1 protein (e.g., misfolded Serpina1 protein), excess production of the Serpina1 protein, by Serpina1 gene variants, Serpina1 gene mutations, by abnormal cleavage of the Serpina1 protein, by abnormal interactions between Serpina1 and other proteins or other endogenous or exogenous substances. A Serpina1 associated disease may be a liver disease and/or a lung disease.

A “liver disease”, as used herein, includes a disease, disorder, or condition affecting the liver and/or its function. A liver disorder can be the result of accumulation of Serpina1 protein in the liver and/or liver cells. Examples of liver disorders include liver disorders resulting from, viral infections, parasitic infections, genetic predisposition, autoimmune diseases, exposure to radiation, exposure to hepatotoxic compounds, mechanical injuries, various environmental toxins, alcohol, acetaminophen, a combination of alcohol and acetaminophen, inhalation anesthetics, niacin, chemotherapeutics, antibiotics, analgesics, antiemetics and the herbal supplement kava, and combinations thereof.

For example, a liver disorder associated with Serpina1 deficiency may occur more often in subjects with one or more copies of certain alleles (e.g., the PIZ, PiM(Malton), and/or PIS alleles). Without wishing to be bound by theory, it is thought that alleles associated with a greater risk of developing an alpha-1 anti-trypsin liver disease encode forms of Serpina1 which are subject to misfolding and are not properly secreted from the hepatocytes. The cellular responses to these misfolded proteins can include the unfolded protein response (UPR), endoplasmic reticulum-associated degradation (ERAD), apoptosis, ER overload response, autophagy, mitochondrial stress and altered hepatocyte function. The injuries to the hepatocytes can lead to symptoms such as, but not limited to, inflammation, cholestasis, fibrosis, cirrhosis, prolonged obstructive jaundice, increased transaminases, portal hypertension and/or hepatocellular carcinoma. Without wishing to be bound by theory, the highly variable clinical course of this disease is suggestive of modifiers or “second hits” as contributors to developing symptoms or progressing in severity.

For example, subjects with a PIZ allele can be more sensitive to Hepatitis C infections or alcohol abuse and more likely to develop a liver disorder if exposed to such factors. Additionally cystic fibrosis (CF) subjects carrying the PIZ allele are at greater risk of developing severe liver disease with portal hypertension. A deficiency of Serpina1 can also cause or contribute to the development of early onset emphysema, necrotizing panniculitis, bronchiectasis, and/or prolonged neonatal jaundice. Some patients having or at risk of having a deficiency of alpha-1-antitrypsin are identified by screening when they have family members affected by an alpha-1-antitrypsin deficiency.

Exemplary liver disorders include, but are not limited to, liver inflammation, chronic liver disease, cirrhosis, liver fibrosis, hepatocellular carcinoma, liver necrosis, steatosis, cholestatis and/or reduction and/or loss of hepatocyte function.

“Cirrhosis” is a pathological condition associated with chronic liver damage that includes extensive fibrosis and regenerative nodules in the liver.

“Fibrosis” is the proliferation of fibroblasts and the formation of scar tissue in the liver.

The phrase “liver function” refers to one or more of the many physiological functions performed by the liver. Such functions include, but are not limited to, regulating blood sugar levels, endocrine regulation, enzyme systems, interconversion of metabolites (e.g., ketone bodies, sterols and steroids and amino acids); manufacturing blood proteins such as fibrinogen, serum albumin, and cholinesterase, erythropoietic function, detoxification, bile formation, and vitamin storage. Several tests to examine liver function are known in the art, including, for example, measuring alanine amino transferase (ALT), alkaline phosphatase, bilirubin, prothrombin, and albumin.

“Therapeutically effective amount,” as used herein, is intended to include the amount of an RNAi agent that, when administered to a patient for treating a Serpina1-associated disease, is sufficient to effect treatment of the disease (e.g., by diminishing, ameliorating or maintaining the existing disease or one or more symptoms of disease). The “therapeutically effective amount” may vary depending on the RNAi agent, how the agent is administered, the disease and its severity and the history, age, weight, family history, genetic makeup, stage of pathological processes mediated by Serpina1 expression, the types of preceding or concomitant treatments, if any, and other individual characteristics of the patient to be treated.

“Prophylactically effective amount,” as used herein, is intended to include the amount of an RNAi agent that, when administered to a subject who does not yet experience or display symptoms of an Serpina1-associated disease, but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylacticaly effective amount” also includes an amount of an RNAi agent that produces some desired local or systemic effect at a reasonable benefit/risk ratio applicable to any treatment. RNAi gents employed in the methods of the present invention may be administered in a sufficient amount to produce a reasonable benefit/risk ratio applicable to such treatment.

The term “sample,” as used herein, includes a collection of similar fluids, cells, or tissues isolated from a subject, as well as fluids, cells, or tissues present within a subject. Examples of biological fluids include blood, serum and serosal fluids, plasma, urine, lymph, cerebrospinal fluid, ocular fluids, saliva, and the like. Tissue samples may include samples from tissues, organs or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In preferred embodiments, a “sample derived from a subject” refers to blood or plasma drawn from the subject. In further embodiments, a “sample derived from a subject” refers to liver tissue (or subcomponents thereof) derived from the subject.

### II. iRNAs of the Invention

Described herein are improved double stranded RNAi agents which inhibit the expression of a Serpina1 gene in a cell, such as a cell within a subject, e.g., a mammal, such as a human having a Serpina1 associated disease, e.g., a liver disease, e.g., chronic liver disease, liver inflammation, cirrhosis, liver fibrosis, and/or hepatocellular carcinoma.

Accordingly, the invention provides double stranded RNAi agents with chemical modifications capable of inhibiting the expression of a target gene (i.e., a Serpina1 gene) in vivo. In certain aspects of the invention, substantially all of the nucleotides of an iRNA of the invention are modified. In other embodiments of the invention, all of the nucleotides of an iRNA of the invention are modified. iRNAs of the invention in which “substantially all of the nucleotides are modified” are largely but not wholly modified and can include not more than 5, 4, 3, 2, or 1 unmodified nucleotides.

The RNAi agent comprises a sense strand and an antisense strand. Each strand of the RNAi agent may range from 12-30 nucleotides in length. For example, each strand may be between 14-30 nucleotides in length, 17-30 nucleotides in length, 19-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as an “RNAi agent.” The duplex region of an RNAi agent may be 12-30 nucleotide pairs in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In one embodiment, the RNAi agent may contain one or more overhang regions and/or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3′-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

Any of the nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. Specific examples of iRNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified iRNA will have a phosphorus atom in its internucleoside backbone.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in iRNAs, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An iRNA of the invention can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

An iRNA of the invention can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

An iRNA of the invention can also be modified to include one or more bicyclic sugar moities. A “bicyclic sugar” is a furanosyl ring modified by the bridging of two atoms. A“bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge. Examples of such 4′ to 2′ bridged bicyclic nucleosides, include but are not limited to 4′-(CH2)-O-2′ (LNA); 4′-(CH2)-S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof; see, e.g., U.S. Pat. No. 7,399,845); 4′-C(CH3)(CH3)-O-2′ (and analogs thereof; see e.g., U.S. Pat. No. 8,278,283); 4′-CH2 N(OCH3)-2′ (and analogs thereof; see e.g., U.S. Pat. No. 8,278,425); 4′-CH2-O-N(CH3)-2′ (see, e.g., U.S. Patent Publication No. 2004/0171570); 4′-CH2 N(R)O-2′, wherein R is H, C1-C12 alkyl, or a protecting group (see, e.g., U.S. Pat. No. 7,427,672); 4′-CH2-C(H)(CH3)-2′ (see, e.g., Chattopadhyaya et al., J. Org. Chem., 2009, 74, 118-134); and 4′-CH2-C(═CH2)-2′ (and analogs thereof; see, e.g., U.S. Pat. No. 8,278,426). The entire contents of each of the foregoing are hereby incorporated herein by reference.

Additional representative U.S. Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference. Any of the foregoing bicyclic nucleosides can be prepared having one or more stereochemical sugar configurations including for example α-L-ribofuranose and β-D-ribofuranose (see WO 99/14226).

An iRNA of the invention can also be modified to include one or more constrained ethyl nucleotides. As used herein, a “constrained ethyl nucleotide” or “cEt” is a locked nucleic acid comprising a bicyclic sugar moiety comprising a 4′-CH(CH3)-0-2′ bridge. In one embodiment, a constrained ethyl nucleotide is in the S conformation referred to herein as “S-cEt.”

An iRNA of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an iRNA of the invention comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-0-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other modifications of an iRNA of the invention include a 5′ phosphate or 5′ phosphate mimic, e.g., a 5′-terminal phosphate or phosphate mimic on the antisense strand of an RNAi agent. Suitable phosphate mimics are disclosed in, for example US Patent Publication No. 2012/0157511, the entire contents of which are incorporated herein by reference.

A. iRNAs Comprising Thermally Destabilizing Modifications.

As described herein, it has been discovered inter alia that off-target effects of dsRNA agents targeting Serpina1 can be reduced or inhibited by incorporating thermally destabilizing nucleotides at certain positions in the antisense strand of the dsRNA. With these thermally destabilizing modifications at certain positions in antisense strand, the dsRNA agents were able to retain gene silencing activity similar to the parent dsRNA while having reduced off-target gene silencing. Further, the number of off-target genes that are down-regulated or up-regulated is also reduced by dsRNA agents comprising these thermally destabilizing modifications when compared to the parent dsRNA.

As such, in one aspect, the invention provides a double stranded RNAi (dsRNA) agent capable of inhibiting expression of a Serpina1 target gene. Generally, the dsRNA agents of the invention show high on-target gene silencing while reducing or minimizing off-target gene silencing and/or toxicity. Without limitations, the dsRNA agents of the invention can be substituted for the dsRNA agents and can be used for in RNA interference based gene silencing techniques, including, but not limited to, in vitro or in vivo applications.

Generally, the dsRNA agent comprises a sense strand (also referred to as passenger strand) and an antisense strand (also referred to as guide strand). Each strand of the dsRNA agent can range from 12-40 nucleotides in length. For example, each strand can be between 14-40 nucleotides in length, 17-37 nucleotides in length, 25-37 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length.

In some embodiments, the antisense strand is of length 18 to 35 nucleotides. In some embodimens, the antisense strand is 21-25, 19-25, 19-21 or 21-23 nucleotides in length. In some particular embodiments, the antisense strand is 23 nucleotides in length. Similar to the antisense strand, the sense strand can be, in some embodiments, 18-35 nucloeitdes in length. In some embodimens, the sense strand is 21-25, 19-25, 19-21 or 21-23 nucleotides in length. In some particular embodiments, the antisense strand is 21 nucleotides in length.

In some embodiments, the sense and antisense strands are independently 19, 20, 21, 22, 23, 24 or 25 nucleotides in length, wherein the antisense strand contains at least one thermally destabilizing nucleotide, and where the at least one thermally destabilizing nucleotide is in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; (vii) the dsRNA comprises a duplex region of 18, 19, 21, 22, 23, 24 or 24 nucleotide pairs in length; and (viii) the dsRNA comprises a blunt end at 5′-end of the sense strand. In some particular embodiments, sense strand is 19, 20 or 21 or 22 nucleotides in length and the antisense strand is 20, 21 or 22 nucleotides in length.

The sense strand and antisense strand typically form a duplex dsRNA. The duplex region of a dsRNA agent may be 12-40 nucleotide pairs in length. For example, the duplex region can be between 14-40 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length.

In some embodiments, the dsRNA agent of the invention has a duplex region of 12-40 nucleotides pairs in length, wherein the antisense strand contains at least one thermally destabilizing nucleotide, and where the at least one thermally destabilizing nucleotide is in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications; and (vii) the dsRNA comprises a blunt end at 5′-end of the antisense strand. In some particular embodiments, the duplex region is 18, 19, 20, 21, 22 or 23 nucleotides pairs in length. In a particular embodiment, the duplex region is 21 nucleotide pairs in length.

In some embodiments, the dsRNA agent of the invention comprises one or more overhang regions and/or capping groups of dsRNA agent at the 3′-end, or 5′-end or both ends of a strand. The overhang can be 1-10 nucleotides in length, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be other sequence. The first and second strands can also be joined, e.g., by additional bases to form a hairpin, or by other non-base linkers.

In some embodiments, the nucleotides in the overhang region of the dsRNA agent of the invention can each independently be a modified or unmodified nucleotide including, but not limited to 2′-sugar modified, such as, 2-F 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA agent of the invention may be phosphorylated. In some embodiments, the overhang region contains two nucleotides having a phosphorothioate between the two nucleotides, where the two nucleotides can be the same or different. In some embodiments, the overhang is present at the 3′-end of the sense strand, antisense strand or both strands. In some embodiments, this 3′-overhang is present in the antisense strand. In some embodiments, this 3′-overhang is present in the sense strand.

The dsRNA agent of the invention may comprise only a single overhang, which can strengthen the interference activity of the dsRNA, without affecting its overall stability. For example, the single-stranded overhang is located at the 3′-terminal end of the sense strand or, alternatively, at the 3′-terminal end of the antisense strand. The dsRNA may also have a blunt end, located at the 5′-end of the antisense strand (or the 3′-end of the sense strand) or vice versa. Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process. For example the single overhang comprises at least two, three, four, five, six, seven, eight, nine, or ten nucleotides in length. In some embodiments, the dsRNA has a 2 nucleotide overhang on the 3′-end of the antisense strand and a blunt end at the 5′-end of the antisense strand.

In some embodiments, one end of the dsRNA is a blunt end and the other end has an overhang, wherein the antisense strand contains at least one thermally destabilizing nucleotide, and where the at least one thermally destabilizing nucleotide is in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; (vii) and the dsRNA comprises a duplex region of 12-40 nucleotide pairs in length. In some embodiments, the overhang is on the 3′-end of the antisense strand and the blunt end is at the 5′-end of the antisense strand. In some particular embodiments, the overhang is 2, 3 or 4-nucleotides in length.

In some embodiments, the dsRNA agent has a duplex region of 19, 21, 22 or 23 nucleotide base pairs in length, wherein one end of the dsRNA is a blunt end and the other end has an overhang, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, five or all six) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications, and optionally the 2 nucleotide overhang is on the 3′-end of the antisense strand and the blunt end is at the 5′-end of the antisense strand. In some embodiments, the overhang is on the 3′-end of the antisense strand and the blunt end is at the 5′-end of the antisense strand.

In some embodiments, the dsRNA agent of the invention may also have two blunt ends, at both ends of the dsRNA duplex.

In some embodiments, the dsRNA has a blunt end at both ends of the duplex, wherein the antisense strand contains at least one thermally destabilizing nucleotide, and where the at least one thermally destabilizing nucleotide is in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; and (vii) the dsRNA comprises a duplex region of 12-40 nucleotide pairs in length.

In some embodiments, the dsRNA agent has a duplex region of 19, 21, 22 or 23 nucleotide base pairs in length and has a blunt end at both ends of the duplex, wherein one end of the dsRNA is a blunt end and the other end has an overhang, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, five or all six) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications.

In some embodiments, the antisense strand comprises at least one (e.g., one, two, three, four, five or more) thermally destabilizing modification of the duplex within the first 9 nucleotide positions of the 5′ region of the antisense strand. In some embodiments, thermally destabilizing modification of the duplex is located in positions 2-9, or preferably positions 4-8, from the 5′-end of the antisense strand. In some further embodiments, the thermally destabilizing modification of the duplex is located at position 6, 7 or 8 from the 5′-end of the antisense strand. In still some further embodiments, the thermally destabilizing modification of the duplex is located at position 7 from the 5′-end of the antisense strand. The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s).

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA).

Exemplified abasic modifications include, but are not limited to the follwoing:

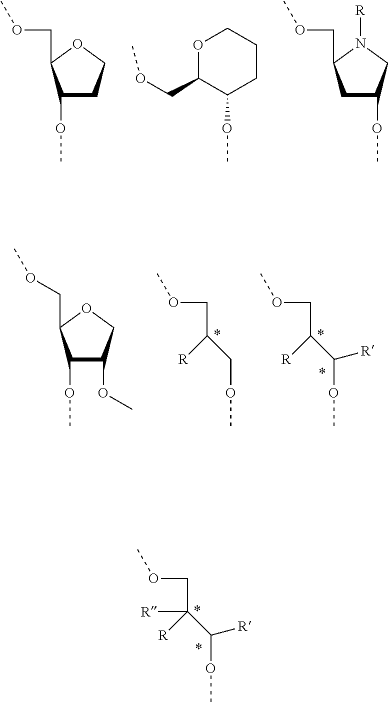

Wherein R=H, Me, Et or OMe; R′=H, Me, Et or OMe; R″=H, Me, Et or OMe

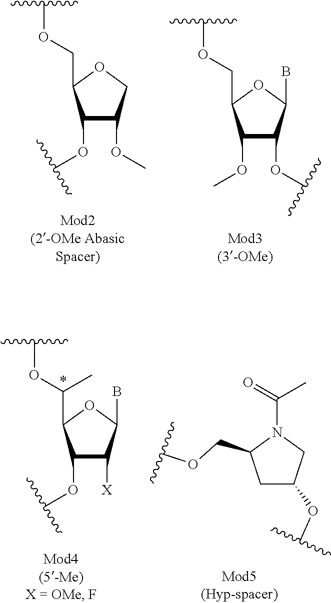

wherein B is a modified or unmodified nucleobase.

Exemplified sugar modifications include, but are not limited to the following:

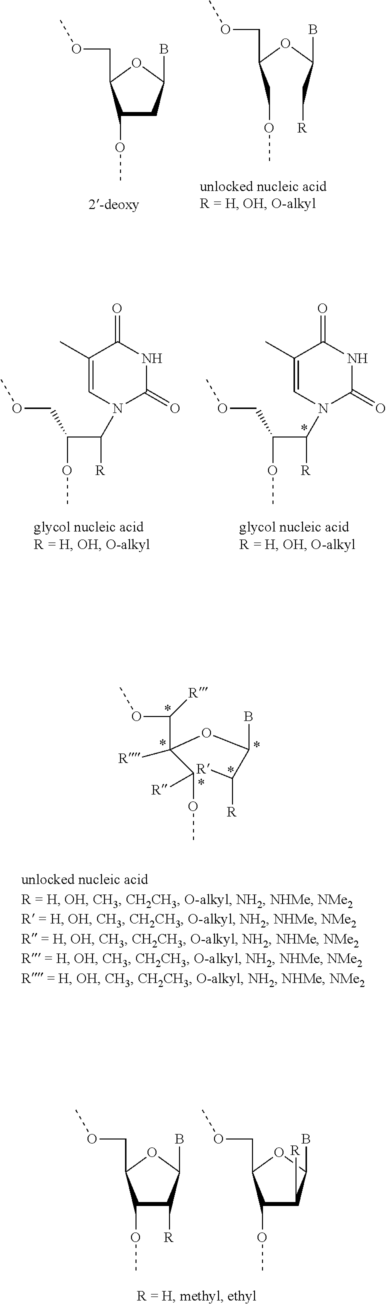

wherein B is a modified or unmodified nucleobase.

In some embodiments the thermally destabilizing modification of the duplex is selected from the group consisting of:

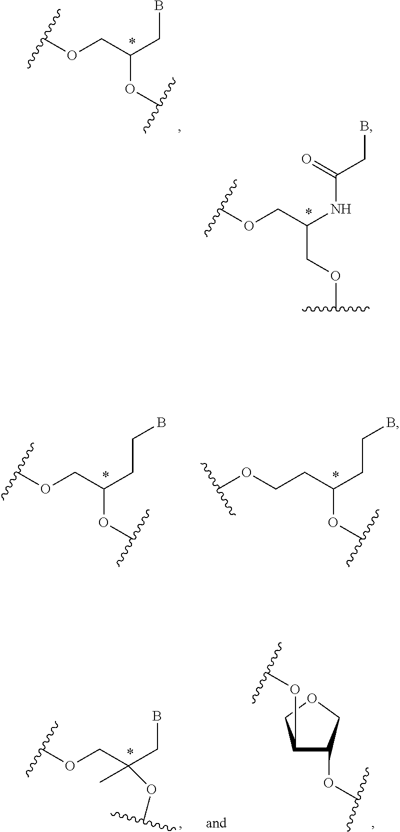

wherein B is a modified or unmodified nucleobase.

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

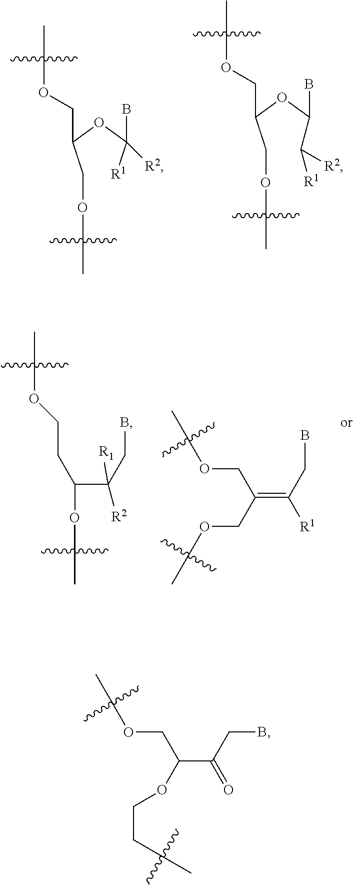

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

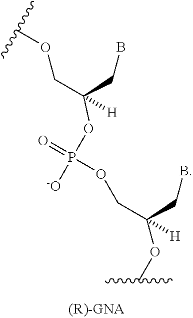

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA agent contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W-C H-bonding to complementary base on the target mRNA, such as:

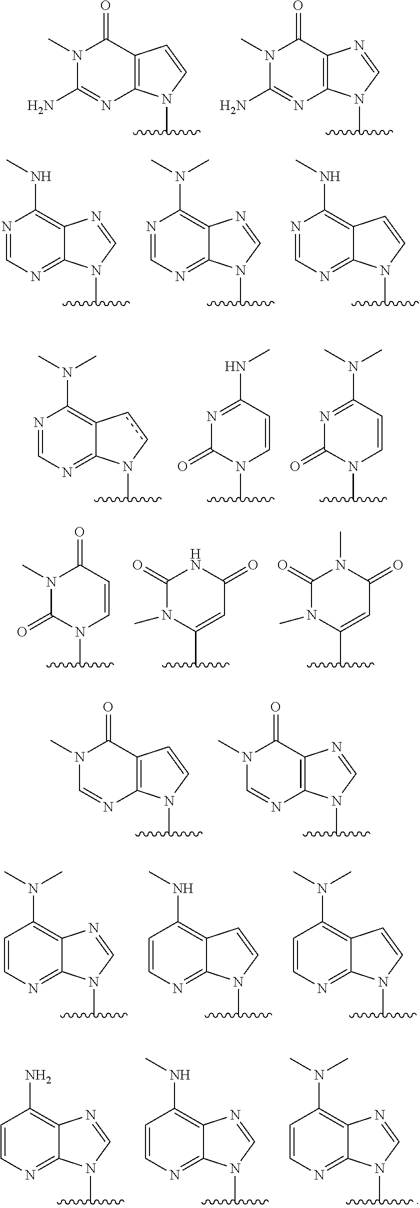

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

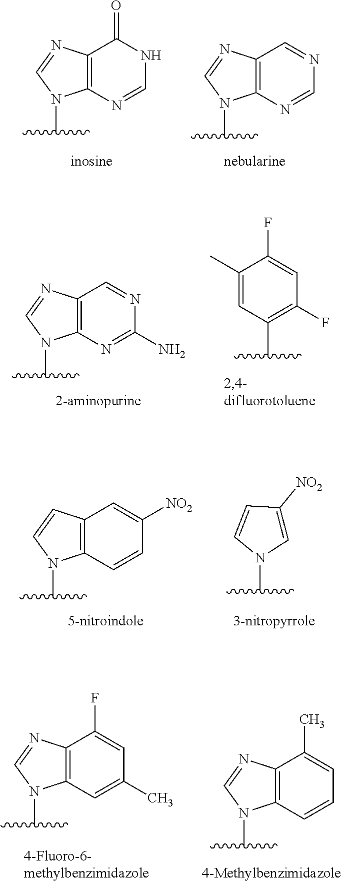

In some embodiments, the thermally destabilizing modification of the duplex in the seed region of the antisense strand includes one or more α-nucleotide complementary to the base on the target mRNA, such as:

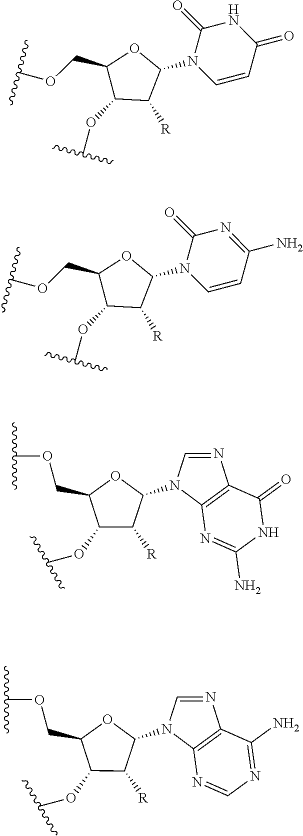

Wherein R is H, OH, OCH₃, F, NH₂, NHMe, NMe₂ or O-alkyl

Exemplary phosphate modifications known to decrease the thermal stability of dsRNA duplexes compared to natural phosphodiester linkages are:

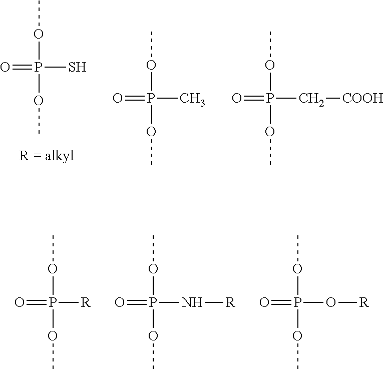

The alkyl for the R group can be a C₁-C₆alkyl. Specific alkyls for the R group include, but are not lmited to methyl, ethyl, propyl, isopropyl, butyl, pentyl and hexyl. In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand and/or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14 and 16 from the 5′-end. In some other embodiments, the antisense comprises stabilizing modifications at positions 2, 6, 14 and 16 from the 5′-end. In still some other embodiments, the antisense comprises stabilizing modifications at positions 2, 14 and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions −1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10 and 11 from the 5′-end. In some other embodiments, the sense strand comprises stabilizing modifications at positions 7, 9, 10 and 11 from the 5′-end. In some embodiments, the sense strand comprises stabilizing modifications at positions opposite or complimentary to positions 11, 12 and 15 of the antisense strand, counting from the 5′-end of the antisense strand. In some other embodiments, the sense strand comprises stabilizing modifications at positions opposite or complimentary to positions 11, 12, 13 and 15 of the antisense strand, counting from the 5′-end of the antisense strand. In some embodiments, the sense strand comprises a block of two, three or four stabilizing modifications.

In some embodiments, the sense strand does not comprise a stabilizing modification in position opposite or complimentary to the thermally destabilizing modification of the duplex in the antisense strand.

Exemplary thermally stabilizing modifications include, but are not limited to 2′-fluoro modifications.

In some embodiments, the dsRNA of the invention comprises at least four (e.g., four, five, six, seven, eight, nine, ten or more) 2′-fluoro nucleotides. Without limitations, the 2′-fluoro nucleotides all can be present in one strand. In some embodiments, both the sense and the antisense strands comprise at least two 2′-fluoro nucleotides. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

In some embodiments, the antisense strand comprises at least two (e.g., two, three, four, five, six, seven, eight, nine, ten or more) 2′-fluoro nucleotides. Without limitations, a 2′-fluoro modification in the antisense strand can be present at any positions. In some embodiments, the antisense comprises 2′-fluoro nucleotides at positions 2, 6, 8, 9, 14 and 16 from the 5′-end. In some other embodiments, the antisense comprises 2′-fluoro nucleotides at positions 2, 6, 14 and 16 from the 5′-end. In still some other embodiments, the antisense comprises 2′-fluoro nucleotides at positions 2, 14 and 16 from the 5′-end.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i.e., at position −1 or +1 from the position of the destabilizing modification. In some embodiments, the antisense strand comprises a 2′-fluoro nucleotide at each of the 5′-end and the 3′-end of the destabilizing modification, i.e., positions −1 and +1 from the position of the destabilizing modification.

In some embodiments, the antisense strand comprises at least two 2′-fluoro nucleotide at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) 2′-fluoro nucleotides. Without limitations, a 2′-fluoro modification in the sense strand can be present at any positions. In some embodiments, the antisense comprises 2′-fluoro nucleotides at positions 7, 10 and 11 from the 5′-end. In some other embodiments, the sense strand comprises 2′-fluoro nucleotides at positions 7, 9, 10 and 11 from the 5′-end. In some embodiments, the sense strand comprises 2′-fluoro nucleotides at positions opposite or complimentary to positions 11, 12 and 15 of the antisense strand, counting from the 5′-end of the antisense strand. In some other embodiments, the sense strand comprises 2′-fluoro nucleotides at positions opposite or complimentary to positions 11, 12, 13 and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complimentary to the thermally destabilizing modification of the duplex in the antisense strand.

In some embodiments, the dsRNA agent of the invention comprises a 21 nucleotides (nt) sense strand and a 23 nucleotides (nt) antisense, wherein the antisense strand contains at least one thermally destabilizing nucleotide, where the at least one thermally destabilizing nucleotide occurs in the seed region of the antisense strand (i.e., at position 2-9 of the 5′-end of the antisense strand), wherein one end of the dsRNA is blunt, while the other end is comprises a 2 nt overhang, and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; and (vii) the dsRNA comprises a blunt end at 5′-end of the antisense strand. Preferably, the 2 nt overhang is at the 3′-end of the antisense.

In some embodiments, the dsRNA agent of the invention comprising a sense and antisense strands, wherein: the sense strand is 25-30 nucleotide residues in length, wherein starting from the 5′ terminal nucleotide (position 1), positions 1 to 23 of said sense strand comprise at least 8 ribonucleotides; antisense strand is 36-66 nucleotide residues in length and, starting from the 3′ terminal nucleotide, at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the antisense strand contains at least one thermally destabilizing nucleotide, where at least one thermally destabilizing nucleotide is in the seed region of the antisense strand (i.e. at position 2-9 of the 5′-end of the antisense strand), For example, the thermally destabilizing nucleotide occurs between positions opposite or complimentary to positions 14-17 of the 5′-end of the sense strand, and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications; and (vii) the dsRNA comprises a duplex region of 12-30 nucleotide pairs in length.

In some embodiments, the dsRNA agent of the invention comprises a sense and antisense strands, wherein said dsRNA agent comprises a sense strand having a length which is at least 25 and at most 29 nucleotides and an antisense strand having a length which is at most 30 nucleotides with the sense strand comprises a modified nucleotide that is susceptible to enzymatic degradation at position 11 from the 5′ end, wherein the 3′ end of said sense strand and the 5′ end of said antisense strand form a blunt end and said antisense strand is 1-4 nucleotides longer at its 3′ end than the sense strand, wherein the duplex region which is at least 25 nucleotides in length, and said antisense strand is sufficiently complementary to a target mRNA along at least 19 nt of said antisense strand length to reduce target gene expression when said dsRNA agent is introduced into a mammalian cell, and wherein dicer cleavage of said dsRNA preferentially results in an siRNA comprising said 3′ end of said antisense strand, thereby reducing expression of the target gene in the mammal, wherein the antisense strand contains at least one thermally destabilizing nucleotide, where the at least one thermally destabilizing nucleotide is in the seed region of the antisense strand (i.e. at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; and (vi) the dsRNA comprises at least four 2′-fluoro modifications; and (vii) the dsRNA has a duplex region of 12-29 nucleotide pairs in length.

In some embodiments, every nucleotide in the sense strand and antisense strand of the dsRNA agent may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. E.g., a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, CeNA, 2′-methoxyethyl, 2′-O-methyl, 2′-O-allyl, 2′-C-allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy, 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with 2′-O-methyl nucleotide, 2′-deoxy nucleotide, 2′-deoxyfluoro nucleotide, 2′-O—N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand.

In some embodiments, the dsRNA agent of the invention comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

The dsRNA agent of the invention may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand and/or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA agent comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA agent comprises 1-10 blocks of two to ten phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA agent comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA agent comprises two blocks of three phosphorothioate or methylphosphonate internucleotide linkages separated by 1, 2, 3, 4, 5, 6, 7, 8, 9, 10, 11, 12, 13, 14, 15 or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA agent comprises two blocks of four phosphorothioate or methylphosphonate internucleotide linkages separated by 1, 2, 3, 4, 5, 6, 7, 8, 9, 10, 11, 12, 13 or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA agent comprises two blocks of five phosphorothioate or methylphosphonate internucleotide linkages separated by 1, 2, 3, 4, 5, 6, 7, 8, 9, 10, 11 or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA agent comprises two blocks of six phosphorothioate or methylphosphonate internucleotide linkages separated by 1, 2, 3, 4, 5, 6, 7, 8, 9 or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA agent comprises two blocks of seven phosphorothioate or methylphosphonate internucleotide linkages separated by 1, 2, 3, 4, 5, 6, 7 or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA agent comprises two blocks of eight phosphorothioate or methylphosphonate internucleotide linkages separated by 1, 2, 3, 4, 5 or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA agent comprises two blocks of nine phosphorothioate or methylphosphonate internucleotide linkages separated by 1, 2, 3 or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA agent of the invention further comprises one or more phosphorothioate or methylphosphonate internucleotide linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleotide linkage at one end or both ends of the sense and/or antisense strand.

In some embodiments, the dsRNA agent of the invention further comprises one or more phosphorothioate or methylphosphonate internucleotide linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleotide linkage at position 8-16 of the duplex region counting from the 5′-end of the sense strand; the dsRNA agent can optionally further comprise one or more phosphorothioate or methylphosphonate internucleotide linkage modification within 1-10 of the termini position(s).

In some embodiments, the dsRNA agent of the invention further comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA agent of the invention further comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA agent of the invention further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA agent of the invention further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA agent of the invention further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end). In some embodiments, the dsRNA agent of the invention further comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA agent of the invention further comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA agent of the invention further comprises one phosphorothioate internucleotide linkage modification within position 1-5 (counting from the 5′-end) of the sense strand, and two phosphorothioate internucleotide linkage modifications at positions 1 and 2 and one phosphorothioate internucleotide linkage modification within positions 18-23 of the antisense strand (counting from the 5′-end).

In some embodiments, the dsRNA agent of the invention further comprises two phosphorothioate internucleotide linkage modifications within position 1-5 (counting from the 5′-end) of the sense strand, and one phosphorothioate internucleotide linkage modification at positions 1 and 2 and two phosphorothioate internucleotide linkage modifications within positions 18-23 of the antisense strand (counting from the 5′-end).

In some embodiments, the dsRNA agent of the invention further comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA agent of the invention further comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA agent of the invention further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA agent of the invention further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA agent of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA agent of the invention further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA agent of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA agent of the invention further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA agent of the invention further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some embodiments, the antisense strand comprises phosphorothioate internucleotide linkages between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3 or 4 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 1, 2, 3 or 4 phosphorothioate internucleotide linkages; (v) the dsRNA comprises at least four 2′-fluoro modifications; (vi) the dsRNA comprises a duplex region of 12-40 nucleotide pairs in length; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3 or 4 phosphorothioate internucleotide linkages; (iii) the sense strand is conjugated with a ligand; (iv) the sense strand comprises 2, 3, 4 or 5 2′-fluoro modifications; (v) the sense strand comprises 3 or 4 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 3 or 4 phosphorothioate internucleotide linkages; (v) the dsRNA comprises at least four 2′-fluoro modifications; (vi) the dsRNA comprises a duplex region of 12-40 nucleotide pairs in length; and (vii) the dsRNA has a blunt end at 5′-end of the antisense strand.

In some embodiments, the dsRNA agent of the invention comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA agent of the invention comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

It has also ben discovered that introducing 4′-modified and/or 5′-modified nucleotide to the 3′-end of a phosphodiester (PO), phosphorothioate (PS), and/or phosphorodithioate (PS2) linkage of a dinucleotide at any position of single stranded or double stranded oligonucleotide can exert steric effect to the internucleotide linkage and, hence, protecting or stabilizing it against nucleases.

In some embodiments, 5′-modified nucleoside is introduced at the 3′-end of a dinucleotide at any position of single stranded or double stranded siRNA. For instance, a 5′-alkylated nucleoside may be introduced at the 3′-end of a dinucleotide at any position of single stranded or double stranded siRNA. The alkyl group at the 5′ position of the ribose sugar can be racemic or chirally pure R or S isomer. An exemplary 5′-alkylated nucleoside is 5′-methyl nucleoside. The 5′-methyl can be either racemic or chirally pure R or S isomer.

In some embodiments, 4′-modified nucleoside is introduced at the 3′-end of a dinucleotide at any position of single stranded or double stranded siRNA. For instance, a 4′-alkylated nucleoside may be introduced at the 3′-end of a dinucleotide at any position of single stranded or double stranded siRNA. The alkyl group at the 4′ position of the ribose sugar can be racemic or chirally pure R or S isomer. An exemplary 4′-alkylated nucleoside is 4′-methyl nucleoside. The 4′-methyl can be either racemic or chirally pure R or S isomer. Alternatively, a 4′-O-alkylated nucleoside may be introduced at the 3′-end of a dinucleotide at any position of single stranded or double stranded siRNA. The 4′-O-alkyl of the ribose sugar can be racemic or chirally pure R or S isomer. An exemplary 4′-O-alkylated nucleoside is 4′-O-methyl nucleoside. The 4′-O-methyl can be either racemic or chirally pure R or S isomer.

In some embodiments, 5′-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 5′-alkylated nucleoside is 5′-methyl nucleoside. The 5′-methyl can be either racemic or chirally pure R or S isomer.

In some embodiments, 4′-alkylated nucleoside is introduced at any position on the sense strand or antisense strand of a dsRNA, and such modification maintains or improves potency of the dsRNA. The 4′-alkyl can be either racemic or chirally pure R or S isomer. An exemplary 4′-alkylated nucleoside is 4′-methyl nucleoside. The 4′-methyl can be either racemic or chirally pure R or S isomer.

In some embodiments, 4′-O-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 4′-O-alkylated nucleoside is 4′-O-methyl nucleoside. The 4′-O-methyl can be either racemic or chirally pure R or S isomer.

In some embodiments, the dsRNA agent of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the dsRNA agent of the invention can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC.

Various publications describe multimeric siRNA which can all be used with the dsRNA of the invention. Such publications include WO2007/091269, U.S. Pat. No. 7,858,769, WO2010/141511, WO2007/117686, WO2009/014887 and WO2011/031520 which are hereby incorporated by their entirely.

As discussed in detail below, the dsRNA agent that contains conjugations of one or more carbohydrate moieties to a dsRNA agent can optimize one or more properties of the dsRNA agent. In many cases, the carbohydrate moiety will be attached to a modified subunit of the dsRNA agent. e.g., the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

In one embodimennt the dsRNA agent of the invention is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

The double stranded RNA (dsRNA) agent of the invention may optionally be conjugated to one or more ligands. The ligand can be attached to the sense strand, antisense strand or both strands, at the 3′-end, 5′-end or both ends. For instance, the ligand may be conjugated to the sense strand, in particular, the 3′-end of the sense strand.

In some embodiments dsRNA agents of the invention are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH2-), 5′-alkyletherphosphonates (R=alkylether-methoxymethyl (MeOCH2-), ethoxymethyl, etc., e.g. RP(OH)(O)—O-5′-). In one example, the modification can in placed in the antisense strand of a dsRNA agent.

### III. iRNAs Conjugated to Ligands

Another modification of the RNA of an iRNA of the invention involves chemically linking to the iRNA one or more ligands, moieties or conjugates that enhance the activity, cellular distribution, or cellular uptake of the iRNA e.g., into a cell. Such moieties include but are not limited to lipid moieties such as a cholesterol moiety (Letsinger et al., Proc. Natl. Acid. Sci. USA, 1989, 86: 6553-6556). In other embodiments, the ligand is cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting or lifetime of an iRNA agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands do not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

In one embodiment, the ligand is an asialoglycoprotein receptor ligand. As used herein an “an asialoglycoprotein receptor ligand” or “ASGPR ligand” is a ligand, such as a carbohydrate ligand (discussed below), that targets a dsRNA agent of the invention to hepatocytes. In certain embodiments, the ligand is a one or more galactose, e.g., an N-acetyl-galactosamine (GalNAc) or one or more GalNAc derivatives.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated iRNAs of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems® (Foster City, Calif.). Any other methods for such synthesis known in the art may additionally or alternatively be employed. It is also known to use similar techniques to prepare other oligonucleotides, such as the phosphorothioates and alkylated derivatives.

In the ligand-conjugated iRNAs and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In certain embodiments, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In other embodiments, the lipid based ligand binds HSA weakly or not at all, such that the conjugate will be preferably distributed to the kidney. Other moieties that target to kidney cells can also be used in place of, or in addition to, the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by target cells such as liver cells. Also included are HSA and low density lipoprotein (LDL).

### B. Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO:29). An RFGF analogue (e.g., amino acid sequence AALLPVLLAAP (SEQ ID NO:30) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO:31) and the Drosophila Antennapedia protein (RQIKIWFQNRRMKWKK (SEQ ID NO:32) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand. Preferred conjugates of this ligand target PECAM-1 or VEGF.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### C. Carbohydrate Conjugates

In some embodiments of the compositions and methods of the invention, an iRNA further comprises a carbohydrate. The carbohydrate conjugated iRNA is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri-, and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In certain embodiments, a carbohydrate conjugate for use in the compositions and methods of the invention is a monosaccharide.

In one embodiment, a carbohydrate conjugate for use in the compositions and methods of the invention is selected from the group consisting of:

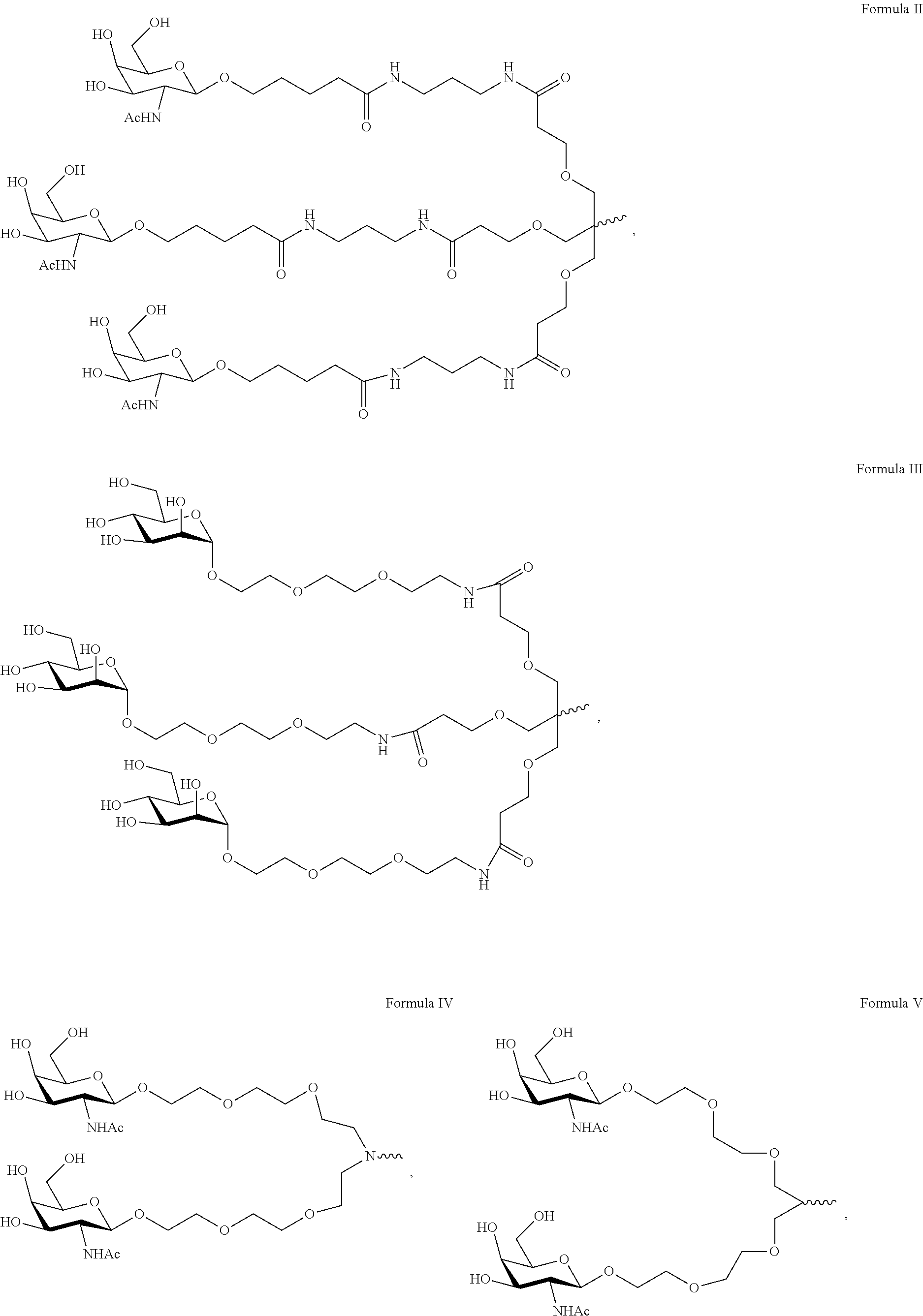

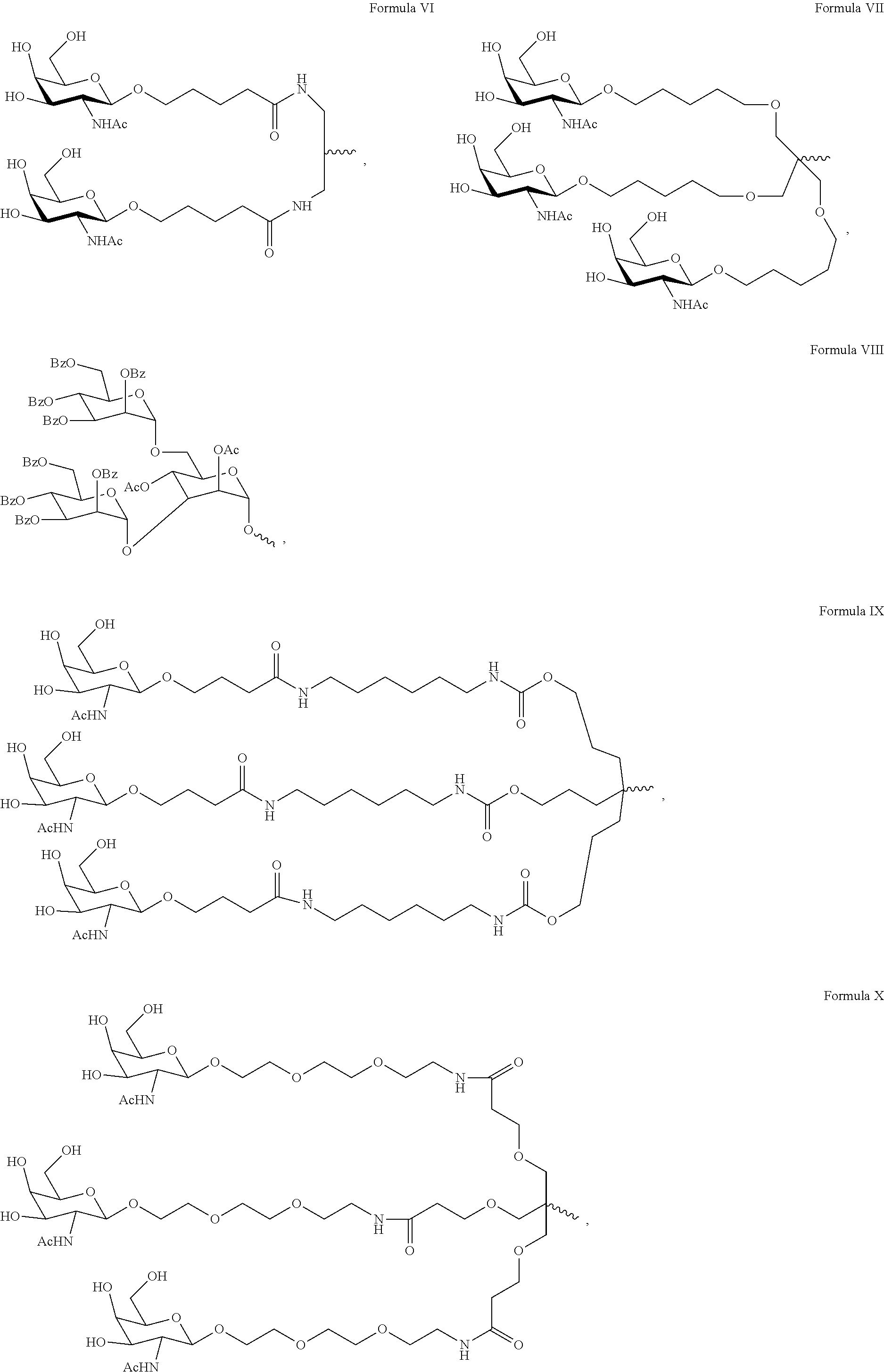

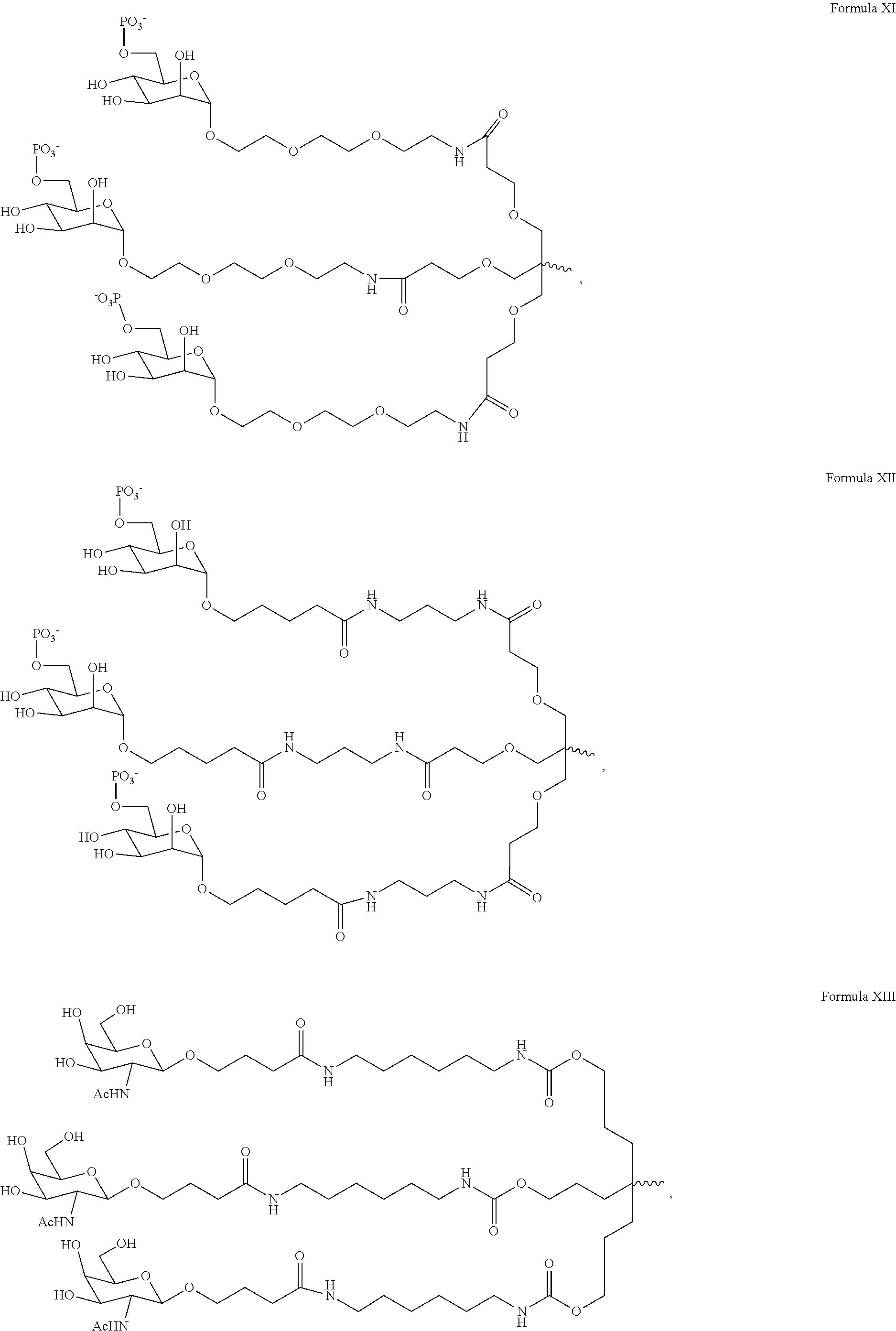

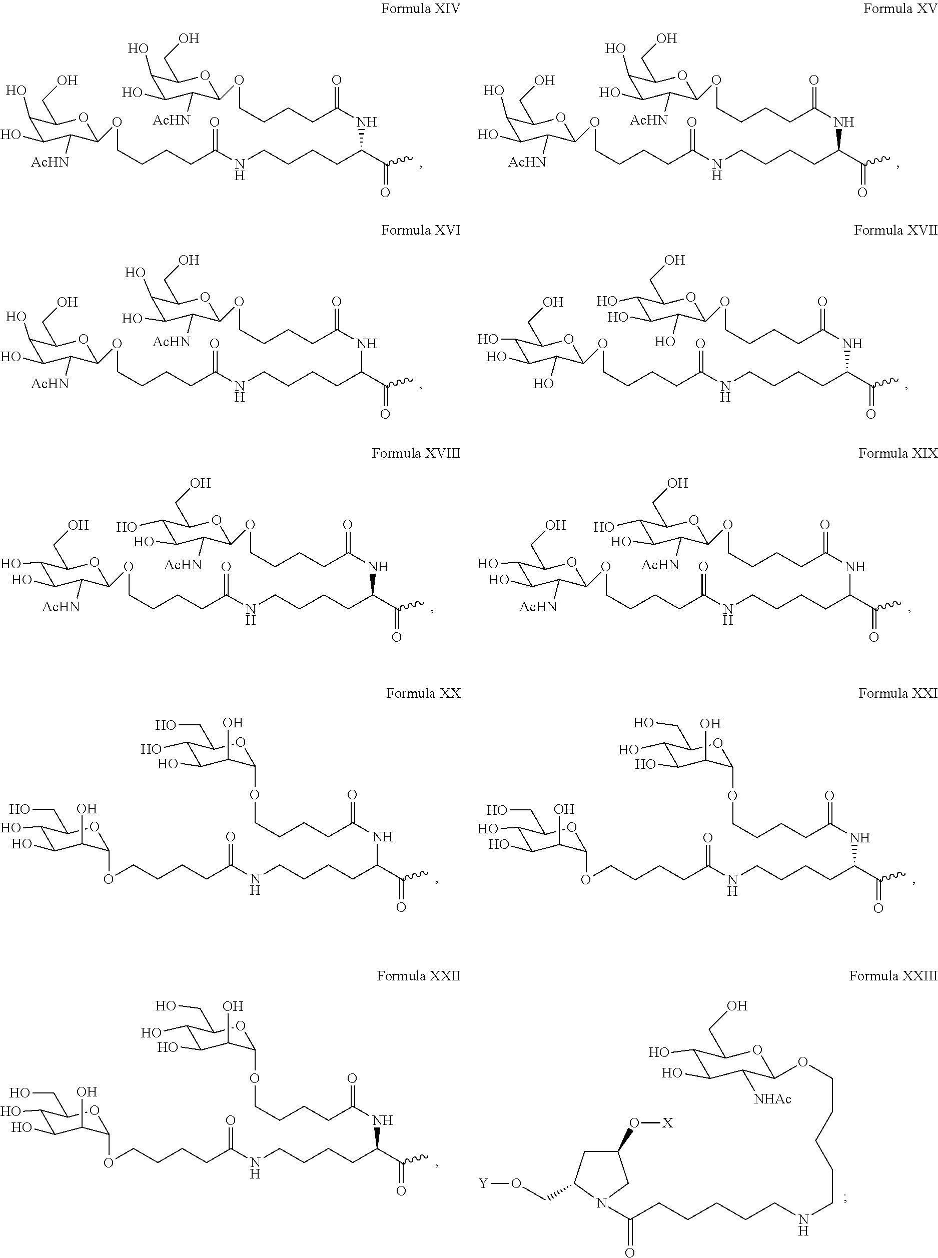

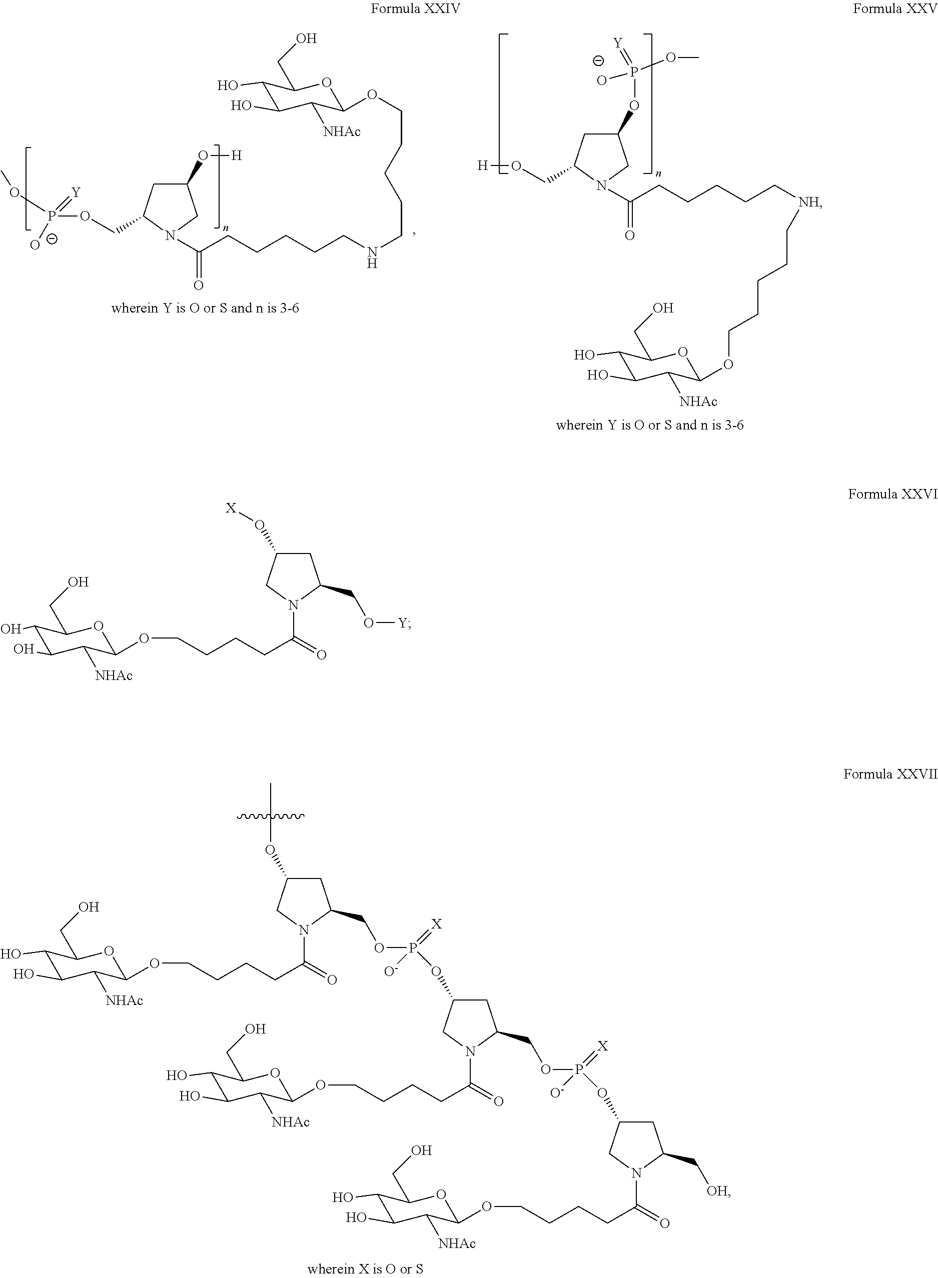

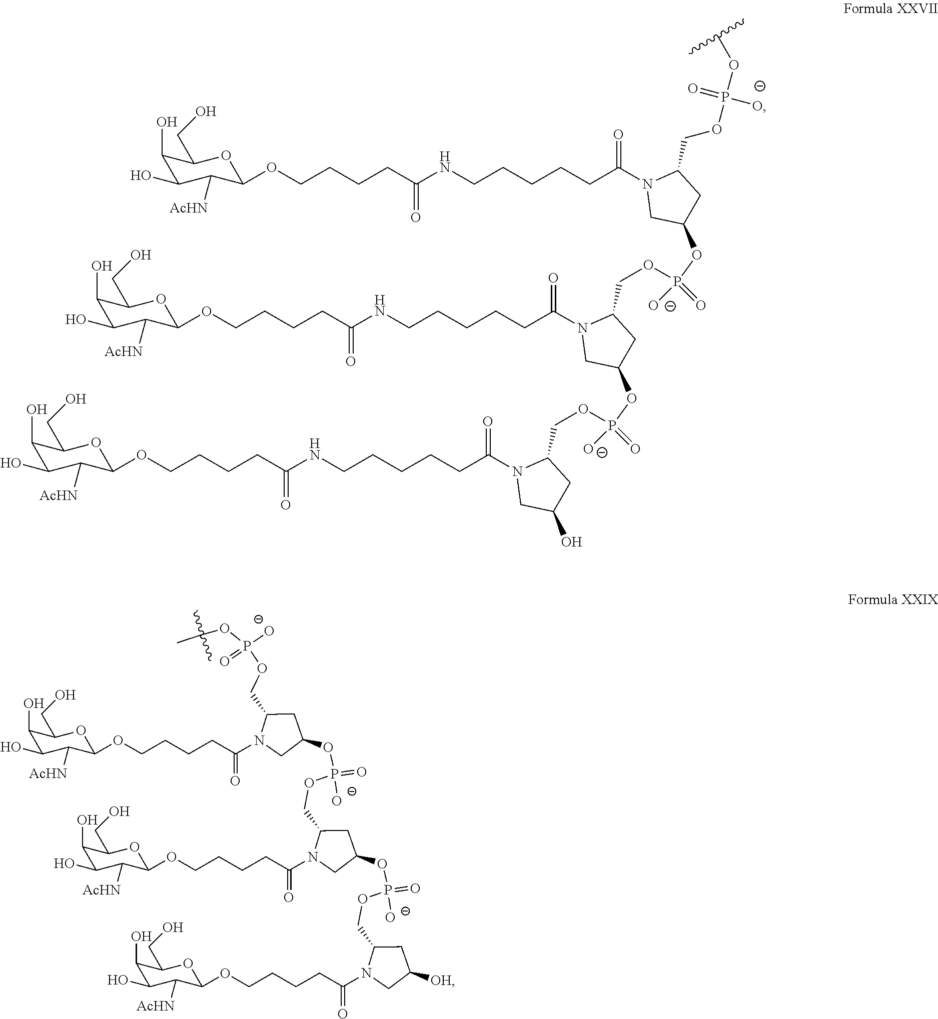

In another embodiment, a carbohydrate conjugate for use in the compositions and methods of the invention is a monosaccharide. In one embodiment, the monosaccharide is an

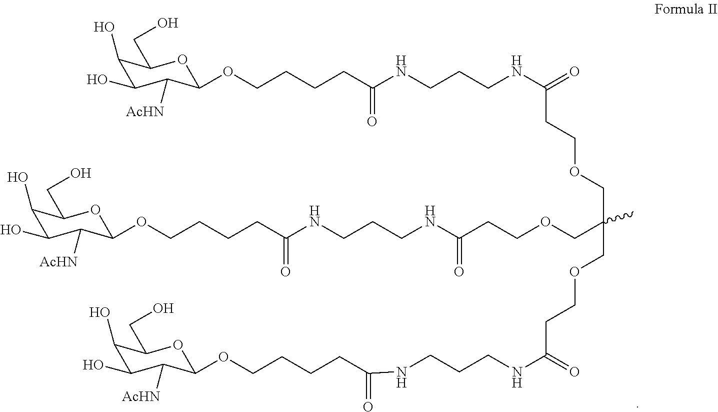 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

Another representative carbohydrate conjugate for use in the embodiments described herein includes, but is not limited to,

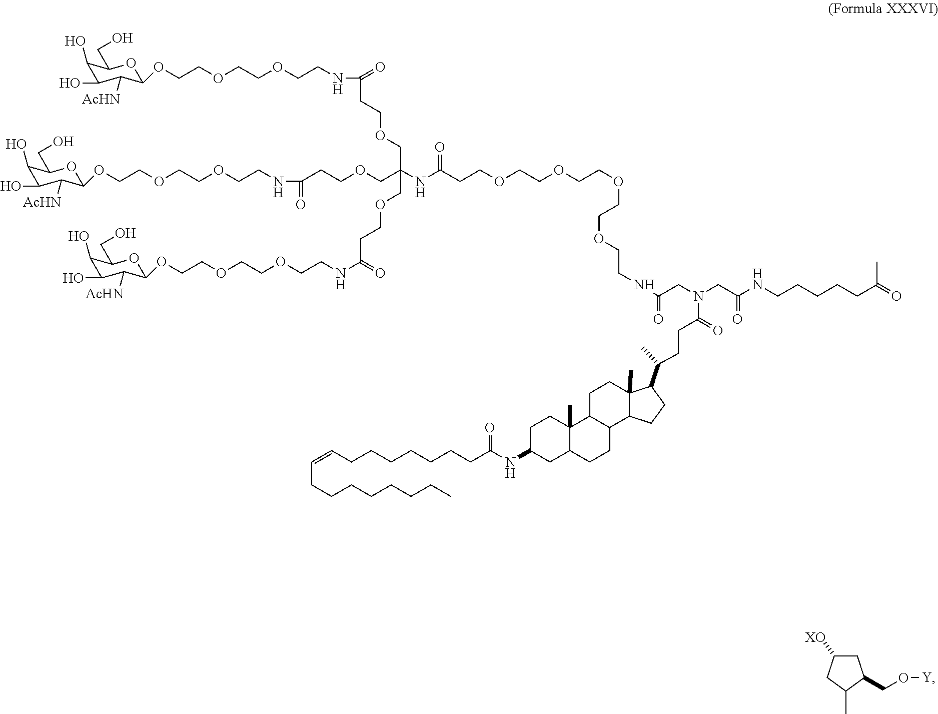

when one of X or Y is an oligonucleotide, the other is a hydrogen.

In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker.

In one embodiment, the double stranded RNAi agents of the invention comprise one GalNAc or GalNAc derivative attached to the iRNA agent, e.g., the 5′ end of the sense strand of a dsRNA agent, or the 5′ end of one or both sense strands of a dual targeting RNAi agent as described herein. In another embodiment, the double stranded RNAi agents of the invention comprise a plurality (e.g., 2, 3, 4, 5, or 6) GalNAc or GalNAc derivatives, each independently attached to a plurality of nucleotides of the double stranded RNAi agent through a plurality of monovalent linkers.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator or a cell permeation peptide.

Additional carbohydrate conjugates and linkers suitable for use in the present invention include those described in PCT Publication Nos. WO 2014/179620 and WO 2014/179627, the entire contents of each of which are incorporated herein by reference.

### D. Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In one embodiment, the linker is between about 1-24 atoms, 2-24, 3-24, 4-24, 5-24, 6-24, 6-18, 7-18, 8-18, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times, or more, or at least 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

i. Redox Cleavable Linking Groups

In certain embodiments, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In other embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, and —O—P(S)(H)—S—. A preferred embodiment is —O—P(O)(OH)—O—. These candidates can be evaluated using methods analogous to those described above.

iii. Acid Cleavable Linking Groups

In other embodiments, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Linking Groups

In other embodiments, a cleavable linker comprises an ester-based cleavable linking group. An ester-based cleavable linking group is cleaved by enzymes such as esterases and amidases in cells. Examples of ester-based cleavable linking groups include, but are not limited to, esters of alkylene, alkenylene and alkynylene groups. Ester cleavable linking groups have the general formula —C(O)O—, or —OC(O)—. These candidates can be evaluated using methods analogous to those described above.

v. Peptide-Based Cleaving Groups

In yet other embodiments, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In some embodiments, an iRNA of the invention is conjugated to a carbohydrate through a linker. Non-limiting examples of iRNA carbohydrate conjugates with linkers of

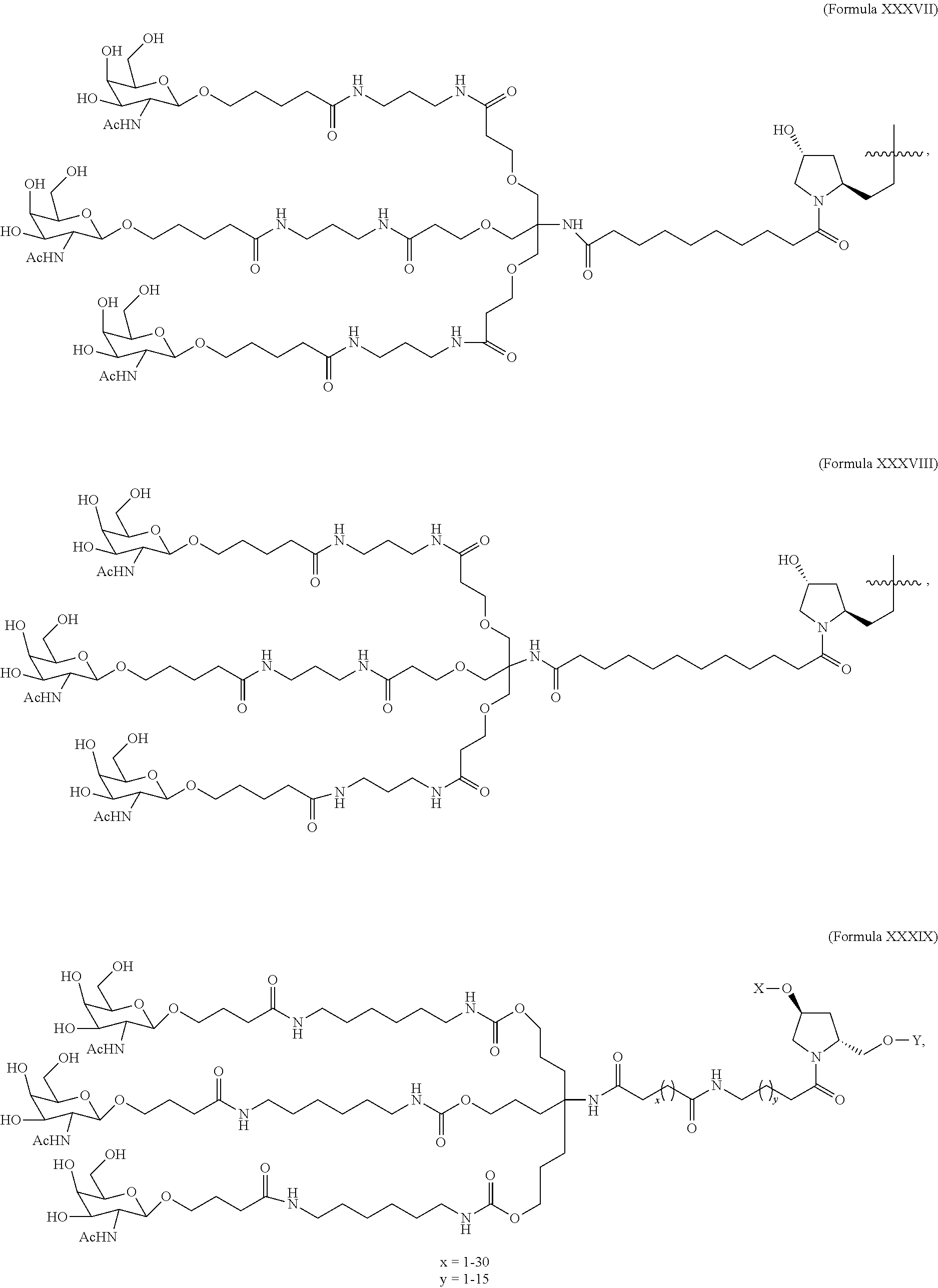

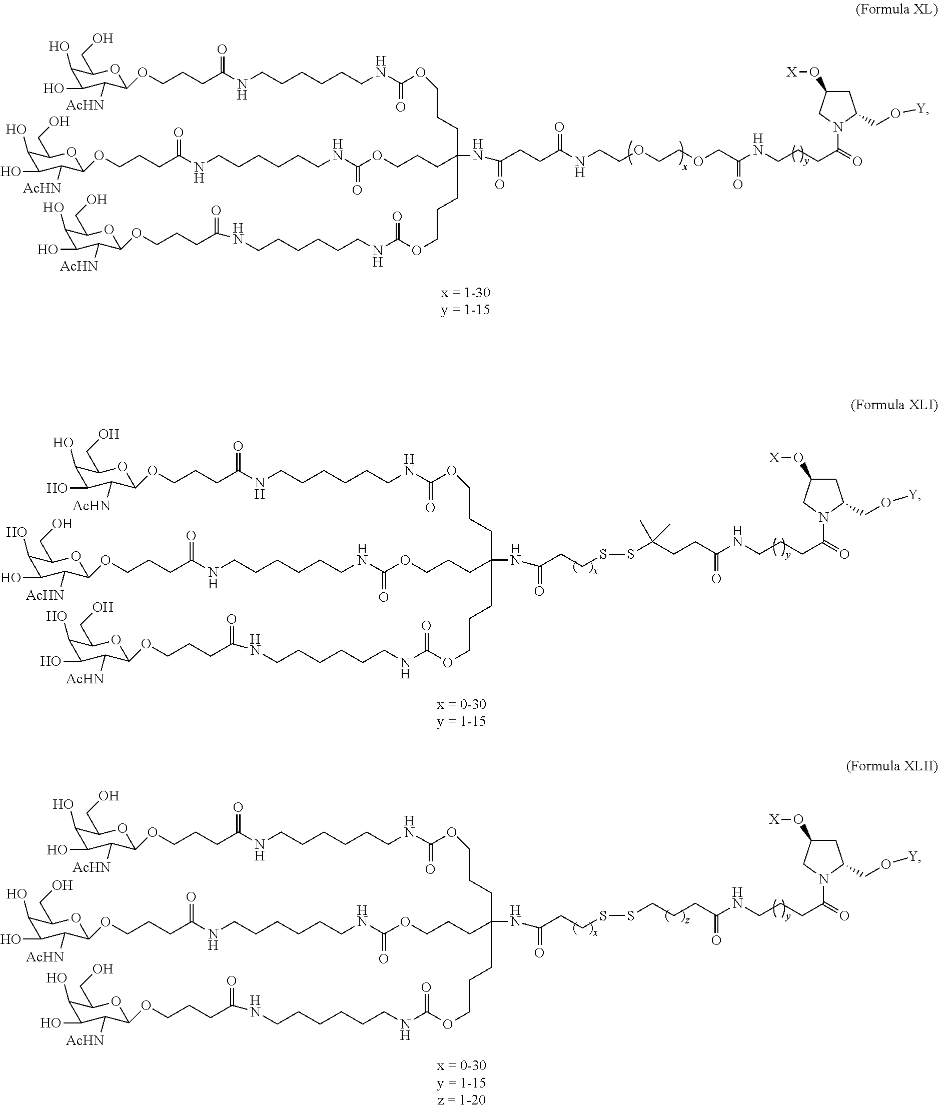

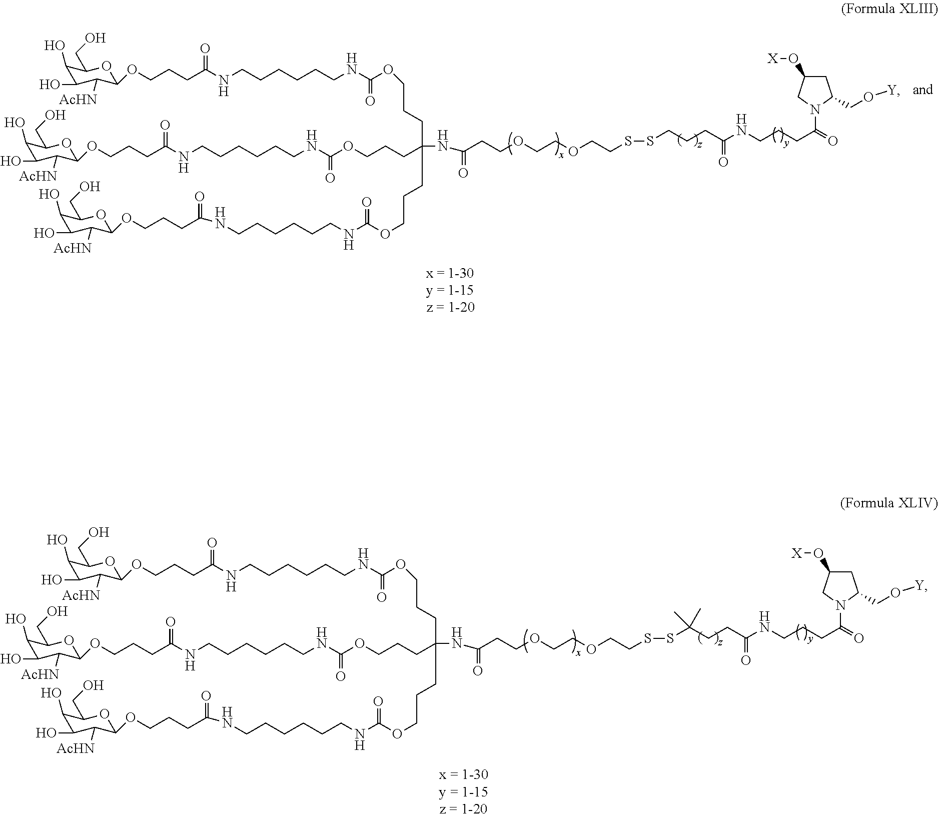

when one of X or Y is an oligonucleotide, the other is a hydrogen.

In certain embodiments of the compositions and methods of the invention, a ligand is one or more “GalNAc” (N-acetylgalactosamine) derivatives attached through a bivalent or trivalent branched linker.

In one embodiment, a dsRNA of the invention is conjugated to a bivalent or trivalent branched linker selected from the group of structures shown in any of formula (XLV)-(XLVI):

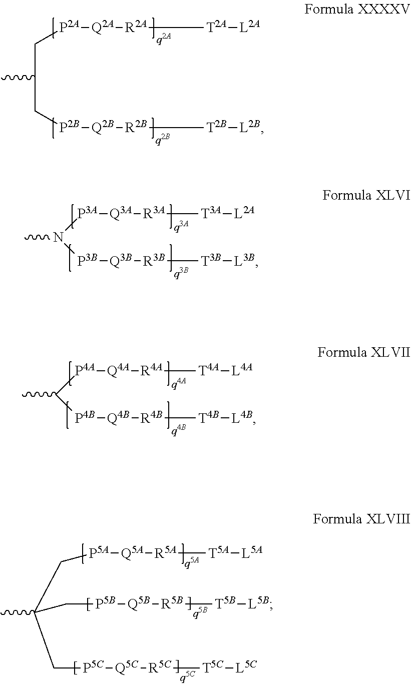

wherein:
q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3b}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3B}, R^{4A}, R^{4B}, R^{5A}, R^{5B}, R^{5D} are each independently for each occurrence absent, NH, O, S, CH₂, C(O)O, C(O)NH, NHCH(R^{a})C(O), —C(O)—CH(R^{a})—NH—, CO, CH═N—O,

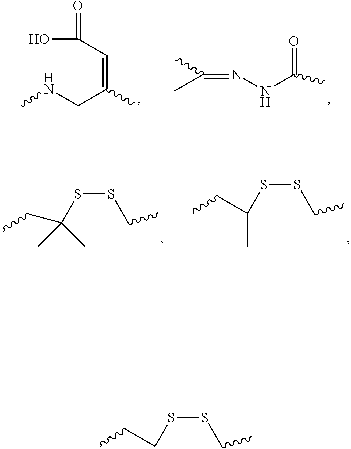

or heterocycyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

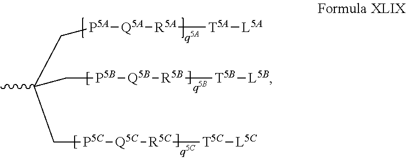

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. Patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNAi agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### IV. Delivery of an iRNA of the Invention

The delivery of an iRNA of the invention to a cell e.g., a cell within a subject, such as a human subject can be achieved in a number of different ways. For example, delivery may be performed by contacting a cell with an iRNA of the invention either in vitro or in vivo. In vivo delivery may also be performed directly by administering a composition comprising an iRNA, e.g., a dsRNA, to a subject. Alternatively, in vivo delivery may be performed indirectly by administering one or more vectors that encode and direct the expression of the iRNA. These alternatives are discussed further below.

In general, any method of delivering a nucleic acid molecule (in vitro or in vivo) can be adapted for use with an iRNA of the invention (see e.g., Akhtar S. and Julian R L. (1992) Trends Cell. Biol. 2(5):139-144 and WO94/02595, which are incorporated herein by reference in their entireties). For in vivo delivery, factors to consider in order to deliver an iRNA molecule include, for example, biological stability of the delivered molecule, prevention of non-specific effects, and accumulation of the delivered molecule in the target tissue. The non-specific effects of an iRNA can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the iRNA molecule to be administered. Several studies have shown successful knockdown of gene products when a dsRNAi agent is administered locally. For example, intraocular delivery of a VEGF dsRNA by intravitreal injection in cynomolgus monkeys (Tolentino, M J, et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of a dsRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering an iRNA systemically for the prevention of an infection, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the iRNA to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an iRNA to an aptamer has been shown to inhibit tumor growth and mediate tumor regression in a mouse model of prostate cancer (McNamara, J O, et al (2006) Nat. Biotechnol. 24:1005-1015). In an alternative embodiment, the iRNA can be delivered using drug delivery systems such as a nanoparticle, a dendrimer, a polymer, liposomes, or a cationic delivery system. Positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H, et al (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R, et al (2003) J. Mol. Biol 327:761-766; Verma, U N, et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N, et al (2003), supra), Oligofectamine, “solid nucleic acid lipid particles” (Zimmermann, T S, et al (2006) Nature 441:111-114), cardiolipin (Chien, P Y, et al (2005) Cancer Gene Ther. 12:321-328; Pal, A, et al (2005) Int J. Oncol. 26:1087-1091), polyethyleneimine (Bonnet M E, et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A, et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605, which is herein incorporated by reference in its entirety.

A. Vector Encoded iRNAs of the Invention

iRNA targeting the Serpina1 gene can be expressed from transcription units inserted into DNA or RNA vectors (see, e.g., Couture, A, et al., TIG. (1996), 12:5-10; Skillern, A, et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

The individual strand or strands of an iRNA can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

iRNA expression vectors are generally DNA plasmids or viral vectors. Expression vectors compatible with eukaryotic cells, preferably those compatible with vertebrate cells, can be used to produce recombinant constructs for the expression of an iRNA as described herein. Eukaryotic cell expression vectors are well known in the art and are available from a number of commercial sources. Typically, such vectors are provided containing convenient restriction sites for insertion of the desired nucleic acid segment. Delivery of iRNA expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an iRNA will generally require regulatory elements, e.g., promoters, enhancers, etc., to ensure the expression of the iRNA in target cells. Other aspects to consider for vectors and constructs are known in the art.

### V. Pharmaceutical Compositions of the Invention

The present invention also includes pharmaceutical compositions and formulations which include the iRNAs of the invention. In one embodiment, provided herein are pharmaceutical compositions containing an iRNA, as described herein, and a pharmaceutically acceptable carrier. The pharmaceutical compositions containing the iRNA are useful for treating a disease or disorder associated with the expression or activity of a Serpina1 gene, e.g., a Serpina1 deficiency-associated disorder, e.g., a Serpina1 deficiency liver disorder. Such pharmaceutical compositions are formulated based on the mode of delivery. One example is compositions that are formulated for systemic administration via parenteral delivery, e.g., by intravenous (IV) delivery. Another example is compositions that are formulated for direct delivery into the brain parenchyma, e.g., by infusion into the brain, such as by continuous pump infusion.

The pharmaceutical compositions comprising RNAi agents of the invention may be, for example, solutions with or without a buffer, or compositions containing pharmaceutically acceptable carriers. Such compositions include, for example, aqueous or crystalline compositions, liposomal formulations, micellar formulations, emulsions, and gene therapy vectors.

In the methods of the invention, the RNAi agent may be administered in a solution. A free RNAi agent may be administered in an unbuffered solution, e.g., in saline or in water. Alternatively, the free siRNA may also be administred in a suitable buffer solution. The buffer solution may comprise acetate, citrate, prolamine, carbonate, or phosphate, or any combination thereof. In a preferred embodiment, the buffer solution is phosphate buffered saline (PBS). The pH and osmolarity of the buffer solution containing the RNAi agent can be adjusted such that it is suitable for administering to a subject.

In some embodiments, the buffer solution further comprises an agent for controlling the osmolarity of the solution, such that the osmolarity is kept at a desired value, e.g., at the physiologic values of the human plasma. Solutes which can be added to the buffer solution to control the osmolarity include, but are not limited to, proteins, peptides, amino acids, non-metabolized polymers, vitamins, ions, sugars, metabolites, organic acids, lipids, or salts. In some embodiments, the agent for controlling the osmolarity of the solution is a salt. In certain embodiments, the agent for controlling the osmolarity of the solution is sodium chloride or potassium chloride.

The pharmaceutical compositions of the invention may be administered in dosages sufficient to inhibit expression of a Serpina1 gene. In general, a suitable dose of an iRNA of the invention will be in the range of about 0.001 to about 200.0 milligrams per kilogram body weight of the recipient per day, generally in the range of about 1 to 50 mg per kilogram body weight per day. For example, the dsRNA can be administered at about 0.01 mg/kg, about 0.05 mg/kg, about 0.5 mg/kg, about 1 mg/kg, about 1.5 mg/kg, about 2 mg/kg, about 3 mg/kg, about 10 mg/kg, about 20 mg/kg, about 30 mg/kg, about 40 mg/kg, or about 50 mg/kg per single dose.

The pharmaceutical composition can be administered once daily, or the iRNA can be administered as two, three, or more sub-doses at appropriate intervals throughout the day or even using continuous infusion or delivery through a controlled release formulation. In that case, the iRNA contained in each sub-dose must be correspondingly smaller in order to achieve the total daily dosage. The dosage unit can also be compounded for delivery over several days, e.g., using a conventional sustained release formulation which provides sustained release of the iRNA over a several day period. Sustained release formulations are well known in the art and are particularly useful for delivery of agents at a particular site, such as could be used with the agents of the present invention. In this embodiment, the dosage unit contains a corresponding multiple of the daily dose.

In other embodiments, a single dose of the pharmaceutical compositions can be long lasting, such that subsequent doses are administered at not more than 3, 4, or 5 day intervals, or at not more than 1, 2, 3, or 4 week intervals. In some embodiments of the invention, a single dose of the pharmaceutical compositions of the invention is administered once per week. In other embodiments of the invention, a single dose of the pharmaceutical compositions of the invention is administered bi-monthly.

The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual iRNAs encompassed by the invention can be made using conventional methodologies or on the basis of in vivo testing using an appropriate animal model, as described elsewhere herein.

Advances in mouse genetics have generated a number of mouse models for the study of various human diseases, such as a liver disorder that would benefit from reduction in the expression of Serpina1. Such models can be used for in vivo testing of iRNA, as well as for determining a therapeutically effective dose. Suitable mouse models are known in the art and include, for example, a mouse containing a transgene expressing human Serpina1.

The pharmaceutical compositions of the present invention can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. The iRNA can be delivered in a manner to target a particular tissue, such as the liver (e.g., the hepatocytes of the liver).

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the iRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). iRNAs featured in the invention can be encapsulated within liposomes or can form complexes thereto, in particular to cationic liposomes. Alternatively, iRNAs can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁20 alkyl ester (e.g., isopropylmyristate IPM), monoglyceride, diglyceride or pharmaceutically acceptable salt thereof). Topical formulations are described in detail in U.S. Pat. No. 6,747,014, which is incorporated herein by reference.

A. iRNA Formulations Comprising Membranous Molecular Assemblies

An iRNA for use in the compositions and methods of the invention can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the iRNA composition. The lipophilic material isolates the aqueous interior from an aqueous exterior, which typically does not include the iRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the iRNA are delivered into the cell where the iRNA can specifically bind to a target RNA and can mediate RNAi. In some cases the liposomes are also specifically targeted, e.g., to direct the iRNA to particular cell types.

A liposome containing a RNAi agent can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The RNAi agent preparation is then added to the micelles that include the lipid component. The cationic groups on the lipid interact with the RNAi agent and condense around the RNAi agent to form a liposome. After condensation, the detergent is removed, e.g., by dialysis, to yield a liposomal preparation of RNAi agent.

If necessary a carrier compound that assists in condensation can be added during the condensation reaction, e.g., by controlled addition. For example, the carrier compound can be a polymer other than a nucleic acid (e.g., spermine or spermidine). pH can also adjusted to favor condensation.

Methods for producing stable polynucleotide delivery vehicles, which incorporate a polynucleotide/cationic lipid complex as structural components of the delivery vehicle, are further described in, e.g., WO 96/37194, the entire contents of which are incorporated herein by reference. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986). Microfluidization can be used when consistently small (50 to 200 nm) and relatively uniform aggregates are desired (Mayhew, et al. Biochim. Biophys. Acta 775:169, 1984). These methods are readily adapted to packaging RNAi agent preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap nucleic acids rather than complex with it. Since both the nucleic acid and the lipid are similarly charged, repulsion rather than complex formation occurs. Nevertheless, some nucleic acid is entrapped within the aqueous interior of these liposomes. pH-sensitive liposomes have been used to deliver nucleic acids encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al. S.T.P. Pharma. Sci., 1994, 4(6) 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver RNAi agents to macrophages.

Further advantages of liposomes include: liposomes obtained from natural phospholipids are biocompatible and biodegradable; liposomes can incorporate a wide range of water and lipid soluble drugs; liposomes can protect encapsulated RNAi agents in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of RNAi agent (see, e.g., Felgner, P. L. et al., Proc. Natl. Acad. Sci., USA 8:7413-7417, 1987 and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer RNAi agent into the skin. In some implementations, liposomes are used for delivering RNAi agent to epidermal cells and also to enhance the penetration of RNAi agent into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with RNAi agent are useful for treating a dermatological disorder.

Liposomes that include iRNA can be made highly deformable. Such deformability can enable the liposomes to penetrate through pore that are smaller than the average radius of the liposome. For example, transfersomes are a type of deformable liposomes. Transferosomes can be made by adding surface edge activators, usually surfactants, to a standard liposomal composition. Transfersomes that include RNAi agent can be delivered, for example, subcutaneously by infection in order to deliver RNAi agent to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transferosomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The iRNA for use in the methods of the invention can also be provided as micellar formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the siRNA composition and at least the alkali metal alkyl sulphate. The first micellar composition is then mixed with at least three micelle forming compounds to form a mixed micellar composition. In another method, the micellar composition is prepared by mixing the siRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### B. Lipid Particles

iRNAs, e.g., dsRNAs of in the invention may be fully encapsulated in a lipid formulation, e.g., a LNP, or other nucleic acid-lipid particle.

As used herein, the term “LNP” refers to a stable nucleic acid-lipid particle. LNPs contain a cationic lipid, a non-cationic lipid, and a lipid that prevents aggregation of the particle (e.g., a PEG-lipid conjugate). LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

The cationic lipid can be, for example, N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1), or a mixture thereof. The cationic lipid can comprise from about 20 mol % to about 50 mol % or about 40 mol % of the total lipid present in the particle.

In another embodiment, the compound 2,2-Dilinoleyl-4-dimethylaminoethyl-[1,3]-dioxolane can be used to prepare lipid-siRNA nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

In one embodiment, the lipid-siRNA particle includes 40% 2,2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 siRNA/Lipid Ratio.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In one embodiment, the lipidoid ND98·4HCl (MW 1487) (see U.S. patent application Ser. No. 12/056,230, filed Mar. 26, 2008, which is incorporated herein by reference), Cholesterol (Sigma-Aldrich), and PEG-Ceramide C16 (Avanti Polar Lipids) can be used to prepare lipid-dsRNA nanoparticles (i.e., LNP01 particles). Stock solutions of each in ethanol can be prepared as follows: ND98, 133 mg/ml; Cholesterol, 25 mg/ml, PEG-Ceramide C16, 100 mg/ml. The ND98, Cholesterol, and PEG-Ceramide C16 stock solutions can then be combined in a, e.g., 42:48:10 molar ratio. The combined lipid solution can be mixed with aqueous dsRNA (e.g., in sodium acetate pH 5) such that the final ethanol concentration is about 35-45% and the final sodium acetate concentration is about 100-300 mM. Lipid-dsRNA nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

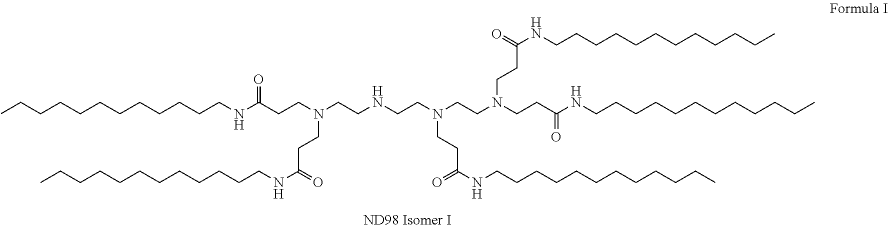 [SMILES: CCCCCCCCCCCCNC(=O)CCN(CCNCCN(CCC(=O)NCCCCCCCCCCCC)CCN(CCC(=O)NCCCCCCCCCCCC)CCC(=O)NCCCCCCCCCCCC)CCC(=O)NCCCCCCCCCCCC]

LNP01 formulations are described, e.g., in International Application Publication No. WO 2008/042973, which is hereby incorporated by reference.

Additional exemplary lipid-dsRNA formulations are described in Table A.

TABLE A

| | | cationic lipid/non-cationic |
|---|---|---|
| | | lipid/cholesterol/PEG-lipid conjugate |
| | Ionizable/Cationic Lipid | Lipid:siRNA ratio |
| LNP-1 | 1,2-Dilinolenyloxy-N,N-dimethylaminopropane | DLinDMA/DPPC/Cholesterol/PEG-CDMA |
| | (DLinDMA) | (57.1/7.1/34.4/1.4) |
| | | lipid:siRNA~7:1 |
| 2-XTC | 2,2-Dilinoleyl-4-dimethylaminoethyl-[1,3]- | XTC/DPPC/Cholesterol/PEG-CDMA |
| | dioxolane (XTC) | 57.1/7.1/34.4/1.4 |
| | | lipid:siRNA~7:1 |
| LNP05 | 2,2-Dilinoleyl-4-dimethylaminoethyl-[1,3]- | XTC/DSPC/Cholesterol/PEG-DMG |
| | dioxolane (XTC) | 57.5/7.5/31.5/3.5 |
| | | lipid:siRNA~6:1 |
| LNP06 | 2,2-Dilinoleyl-4-dimethylaminoethyl-[1,3]- | XTC/DSPC/Cholesterol/PEG-DMG |
| | dioxolane (XTC) | 57.5/7.5/31.5/3.5 |
| | | lipid:siRNA~11:1 |
| LNP07 | 2,2-Dilinoleyl-4-dimethylaminoethyl-[1,3]- | XTC/DSPC/Cholesterol/PEG-DMG |
| | dioxolane (XTC) | 60/7.5/31/1.5, |
| | | lipid:siRNA~6:1 |
| LNP08 | 2,2-Dilinoleyl-4-dimethylaminoethyl-[1,3]- | XTC/DSPC/Cholesterol/PEG-DMG |
| | dioxolane (XTC) | 60/7.5/31/1.5, |
| | | lipid:siRNA~11:1 |
| LNP09 | 2,2-Dilinoleyl-4-dimethylaminoethyl-[1,3]- | XTC/DSPC/Cholesterol/PEG-DMG |
| | dioxolane (XTC) | 50/10/38.5/1.5 |
| | | Lipid:siRNA 10:1 |
| LNP10 | (3aR,5s,6aS)-N,N-dimethyl-2,2-di((9Z,12Z)- | ALN100/DSPC/Cholesterol/PEG-DMG |
| | octadeca-9,12-dienyl)tetrahydro-3aH- | 50/10/38.5/1.5 |
| | cyclopenta[d][1,3]dioxol-5-amine (ALN100) | Lipid:siRNA 10:1 |
| LNP11 | (6Z,9Z,28Z,31Z)-heptatriaconta-6,9,28,31- | MC-3/DSPC/Cholesterol/PEG-DMG |
| | tetraen-19-yl 4-(dimethylamino)butanoate | 50/10/38.5/1.5 |
| | (MC3) | Lipid:siRNA 10:1 |
| LNP12 | 1,1′-(2-(4-(2-((2-(bis(2- | Tech G1/DSPC/Cholesterol/PEG-DMG |
| | hydroxydodecyl)amino)ethyl)(2- | 50/10/38.5/1.5 |
| | hydroxydodecyl)amino)ethyl)piperazin-1- | Lipid:siRNA 10:1 |
| | yl)ethylazanediyl)didodecan-2-ol (Tech G1) | |
| LNP13 | XTC | XTC/DSPC/Chol/PEG-DMG |
| | | 50/10/38.5/1.5 |
| | | Lipid:siRNA:33:1 |
| LNP14 | MC3 | MC3/DSPC/Chol/PEG-DMG |
| | | 40/15/40/5 |
| | | Lipid:siRNA:11:1 |
| LNP15 | MC3 | MC3/DSPC/Chol/PEG-DSG/GalNAc-PEG-DSG |
| | | 50/10/35/4.5/0.5 |
| | | Lipid:siRNA:11:1 |
| LNP16 | MC3 | MC3/DSPC/Chol/PEG-DMG |
| | | 50/10/38.5/1.5 |
| | | Lipid:siRNA:7:1 |
| LNP17 | MC3 | MC3/DSPC/Chol/PEG-DSG |
| | | 50/10/38.5/1.5 |
| | | Lipid:siRNA:10:1 |
| LNP18 | MC3 | MC3/DSPC/Chol/PEG-DMG |
| | | 50/10/38.5/1.5 |
| | | Lipid:siRNA:12:1 |
| LNP19 | MC3 | MC3/DSPC/Chol/PEG-DMG |
| | | 50/10/35/5 |
| | | Lipid:siRNA:8:1 |
| LNP20 | MC3 | MC3/DSPC/Chol/PEG-DPG |
| | | 50/10/38.5/1.5 |
| | | Lipid:siRNA:10:1 |
| LNP21 | C12-200 | C12-200/DSPC/Chol/PEG-DSG |
| | | 50/10/38.5/1.5 |
| | | Lipid:siRNA:7:1 |
| LNP22 | XTC | XTC/DSPC/Chol/PEG-DSG |
| | | 50/10/38.5/1.5 |
| | | Lipid:siRNA:10:1 |

DSPC: distearoylphosphatidylcholine
DPPC: dipalmitoylphosphatidylcholine
PEG-DMG: PEG-didimyristoyl glycerol (C14-PEG, or PEG-C14) (PEG with avg mol wt of 2000)
PEG-DSG: PEG-distyryl glycerol (C18-PEG, or PEG-C18) (PEG with avg mol wt of 2000)
PEG-CDMA: PEG-carbamoyl-1,2-dimyristyloxypropylamine (PEG with avg mol wt of 2000)

LNP (1,2-Dilinolenyloxy-N,N-dimethylaminopropane (DLinDMA)) comprising formulations are described in International Publication No. WO2009/127060, filed Apr. 15, 2009, which is hereby incorporated by reference.

XTC comprising formulations are described, e.g., in U.S. Provisional Ser. No. 61/148,366, filed Jan. 29, 2009; U.S. Provisional Ser. No. 61/156,851, filed Mar. 2, 2009; U.S. Provisional Serial No. filed Jun. 10, 2009; U.S. Provisional Ser. No. 61/228,373, filed Jul. 24, 2009; U.S. Provisional Ser. No. 61/239,686, filed Sep. 3, 2009, and International Application No. PCT/US2010/022614, filed Jan. 29, 2010, which are hereby incorporated by reference.

MC3 comprising formulations are described, e.g., in U.S. Publication No. 2010/0324120, filed Jun. 10, 2010, the entire contents of which are hereby incorporated by reference.

ALNY-100 comprising formulations are described, e.g., International patent application number PCT/US09/63933, filed on Nov. 10, 2009, which is hereby incorporated by reference.

C12-200 comprising formulations are described in U.S. Provisional Ser. No. 61/175,770, filed May 5, 2009 and International Application No. PCT/US10/33777, filed May 5, 2010, which are hereby incorporated by reference.

### Synthesis of Ionizable/Cationic Lipids

Any of the compounds, e.g., cationic lipids and the like, used in the nucleic acid-lipid particles of the invention can be prepared by known organic synthesis techniques, including the methods described in more detail in the Examples. All substituents are as defined below unless indicated otherwise.

“Alkyl” means a straight chain or branched, noncyclic or cyclic, saturated aliphatic hydrocarbon containing from 1 to 24 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like.

“Alkenyl” means an alkyl, as defined above, containing at least one double bond between adjacent carbon atoms. Alkenyls include both cis and trans isomers. Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

“Alkynyl” means any alkyl or alkenyl, as defined above, which additionally contains at least one triple bond between adjacent carbons. Representative straight chain and branched alkynyls include acetylenyl, propynyl, 1-butynyl, 2-butynyl, 1-pentynyl, 2-pentynyl, 3-methyl-1 butynyl, and the like.

“Acyl” means any alkyl, alkenyl, or alkynyl wherein the carbon at the point of attachment is substituted with an oxo group, as defined below. For example, —C(═O)alkyl, —C(═O)alkenyl, and —C(═O)alkynyl are acyl groups.

“Heterocycle” means a 5- to 7-membered monocyclic, or 7- to 10-membered bicyclic, heterocyclic ring which is either saturated, unsaturated, or aromatic, and which contains from 1 or 2 heteroatoms independently selected from nitrogen, oxygen and sulfur, and wherein the nitrogen and sulfur heteroatoms can be optionally oxidized, and the nitrogen heteroatom can be optionally quaternized, including bicyclic rings in which any of the above heterocycles are fused to a benzene ring. The heterocycle can be attached via any heteroatom or carbon atom. Heterocycles include heteroaryls as defined below. Heterocycles include morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The terms “optionally substituted alkyl”, “optionally substituted alkenyl”, “optionally substituted alkynyl”, “optionally substituted acyl”, and “optionally substituted heterocycle” means that, when substituted, at least one hydrogen atom is replaced with a substituent. In the case of an oxo substituent (═O) two hydrogen atoms are replaced. In this regard, substituents include oxo, halogen, heterocycle, —CN, —ORx, —NRxRy, —NRxC(═O)Ry, —NRxSO2Ry, —C(═O)Rx, —C(═O)ORx, —C(═O)NRxRy, —SOnRx and —SOnNRxRy, wherein n is 0, 1 or 2, Rx and Ry are the same or different and independently hydrogen, alkyl or heterocycle, and each of said alkyl and heterocycle substituents can be further substituted with one or more of oxo, halogen, —OH, —CN, alkyl, —ORx, heterocycle, —NRxRy, —NRxC(═O)Ry, —NRxSO2Ry, —C(═O)Rx, —C(═O)ORx, —C(═O)NRxRy, —SOnRx and —SOnNRxRy.

“Halogen” means fluoro, chloro, bromo and iodo.

In some embodiments, the methods of the invention can require the use of protecting groups. Protecting group methodology is well known to those skilled in the art (see, for example, Protective Groups in Organic Synthesis, Green, T. W. et al., Wiley-Interscience, New York City, 1999). Briefly, protecting groups within the context of this invention are any group that reduces or eliminates unwanted reactivity of a functional group. A protecting group can be added to a functional group to mask its reactivity during certain reactions and then removed to reveal the original functional group. In some embodiments an “alcohol protecting group” is used. An “alcohol protecting group” is any group which decreases or eliminates unwanted reactivity of an alcohol functional group. Protecting groups can be added and removed using techniques well known in the art.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which dsRNAs featured in the invention are administered in conjunction with one or more penetration enhancer surfactants and chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the invention can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, US Publn. No. 20030027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions can be generated from a variety of components that include, but are not limited to, preformed liquids, self-emulsifying solids and self-emulsifying semisolids. Particularly preferred are formulations that target the liver when treating hepatic disorders such as hepatic carcinoma.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention can be formulated into any of many possible dosage forms such as, but not limited to, tablets, capsules, gel capsules, liquid syrups, soft gels, suppositories, and enemas. The compositions of the present invention can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

### C. Additional Formulations

i. Emulsions

The compositions of the present invention can be prepared and formulated as emulsions. Emulsions are typically heterogeneous systems of one liquid dispersed in another in the form of droplets usually exceeding 0.1 m in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either the aqueous phase, oily phase or itself as a separate phase. Pharmaceutical excipients such as emulsifiers, stabilizers, dyes, and antioxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

ii. Microemulsions

In one embodiment of the present invention, the compositions of iRNAs and nucleic acids are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO0310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or iRNAs. Microemulsions have also been effective in the transdermal delivery of active components in both cosmetic and pharmaceutical applications. It is expected that the microemulsion compositions and formulations of the present invention will facilitate the increased systemic absorption of iRNAs and nucleic acids from the gastrointestinal tract, as well as improve the local cellular uptake of iRNAs and nucleic acids.

Microemulsions of the present invention can also contain additional components and additives such as sorbitan monostearate (Grill 3), Labrasol, and penetration enhancers to improve the properties of the formulation and to enhance the absorption of the iRNAs and nucleic acids of the present invention. Penetration enhancers used in the microemulsions of the present invention can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

iii. Microparticles

An RNAi agent of the invention may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present invention employs various penetration enhancers to effect the efficient delivery of nucleic acids, particularly iRNAs, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, NY, 2002; Lee et al., Critical Reviews in Therapeutic Drug Carrier Systems, 1991, p. 92). Each of the above mentioned classes of penetration enhancers are described below in greater detail.

Surfactants (or “surface-active agents”) are chemical entities which, when dissolved in an aqueous solution, reduce the surface tension of the solution or the interfacial tension between the aqueous solution and another liquid, with the result that absorption of iRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, NY, 2002; Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₂₀ alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, MA, 2006; Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (see e.g., Malmsten, M. Surfactants and polymers in drug delivery, Informa Health Care, New York, NY, 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, NY, 2002; Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present invention, can be defined as compounds that remove metallic ions from solution by forming complexes therewith, with the result that absorption of iRNAs through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, MA, 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of iRNAs through the alimentary mucosa (see e.g., Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of iRNAs at the cellular level can also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs. Examples of commercially available transfection reagents include, for example Lipofectamine™ (Invitrogen; Carlsbad, CA), Lipofectamine 2000™ (Invitrogen; Carlsbad, CA), 293fectin™ (Invitrogen; Carlsbad, CA), Cellfectin™ (Invitrogen; Carlsbad, CA), DMRIE-C™ (Invitrogen; Carlsbad, CA), FreeStyle™ MAX (Invitrogen; Carlsbad, CA), Lipofectamine™ 2000 CD (Invitrogen; Carlsbad, CA), Lipofectamine™ (Invitrogen; Carlsbad, CA), RNAiMAX (Invitrogen; Carlsbad, CA), Oligofectamine™ (Invitrogen; Carlsbad, CA), Optifect™ (Invitrogen; Carlsbad, CA), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, WI), TransFast™ Transfection Reagent (Promega; Madison, WI), Tfx™-20 Reagent (Promega; Madison, WI), Tfx™-50 Reagent (Promega; Madison, WI), DreamFect™ (OZ Biosciences; Marseille, France), EcoTransfect (OZ Biosciences; Marseille, France), TransPass^{a} D1 Transfection Reagent (New England Biolabs; Ipswich, MA, USA), LyoVec™/LipoGen™ (Invitrogen; San Diego, CA, USA), PerFectin Transfection Reagent (Genlantis; San Diego, CA, USA), NeuroPORTER Transfection Reagent (Genlantis; San Diego, CA, USA), GenePORTER Transfection reagent (Genlantis; San Diego, CA, USA), GenePORTER 2 Transfection reagent (Genlantis; San Diego, CA, USA), Cytofectin Transfection Reagent (Genlantis; San Diego, CA, USA), BaculoPORTER Transfection Reagent (Genlantis; San Diego, CA, USA), TroganPORTER™ transfection Reagent (Genlantis; San Diego, CA, USA), RiboFect (Bioline; Taunton, MA, USA), PlasFect (Bioline; Taunton, MA, USA), UniFECTOR (B-Bridge International; Mountain View, CA, USA), SureFECTOR (B-Bridge International; Mountain View, CA, USA), or HiFect™ (B-Bridge International, Mountain View, CA, USA), among others.

Other agents can be utilized to enhance the penetration of the administered nucleic acids, including glycols such as ethylene glycol and propylene glycol, pyrrols such as 2-pyrrol, azones, and terpenes such as limonene and menthone.

v. Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′ isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

vi. Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

vii. Other Components

The compositions of the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions can contain substances which increase the viscosity of the suspension including, for example, sodium carboxymethylcellulose, sorbitol and/or dextran. The suspension can also contain stabilizers.

In some embodiments, pharmaceutical compositions featured in the invention include (a) one or more iRNA compounds and (b) one or more agents which function by a non-RNAi mechanism and which are useful in treating a bleeding disorder. Examples of such agents include, but are not lmited to an anti-inflammatory agent, anti-steatosis agent, anti-viral, and/or anti-fibrosis agent. In addition, other substances commonly used to protect the liver, such as silymarin, can also be used in conjunction with the iRNAs described herein. Other agents useful for treating liver diseases include telbivudine, entecavir, and protease inhibitors such as telaprevir and other disclosed, for example, in Tung et al., U.S. Application Publication Nos. 2005/0148548, 2004/0167116, and 2003/0144217; and in Hale et al., U.S. Application Publication No. 2004/0127488.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions featured herein in the invention lies generally within a range of circulating concentrations that include the ED50 with little or no toxicity. The dosage can vary within this range depending upon the dosage form employed and the route of administration utilized. For any compound used in the methods featured in the invention, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the iRNAs featured in the invention can be administered in combination with other known agents effective in treatment of pathological processes mediated by Serpina1 expression. In any event, the administering physician can adjust the amount and timing of iRNA administration on the basis of results observed using standard measures of efficacy known in the art or described herein.

### VI. Methods for Inhibiting Serpina1 Expression

The present invention provides methods of inhibiting expression of a Serpina1 in a cell. The methods include contacting a cell with an RNAi agent, e.g., a double stranded RNAi agent, in an amount effective to inhibit expression of the Serpina1 in the cell, thereby inhibiting expression of the Serpina1 in the cell.

Contacting of a cell with a double stranded RNAi agent may be done in vitro or in vivo. Contacting a cell in vivo with the RNAi agent includes contacting a cell or group of cells within a subject, e.g., a human subject, with the RNAi agent. Combinations of in vitro and in vivo methods of contacting are also possible. Contacting may be direct or indirect, as discussed above. Furthermore, contacting a cell may be accomplished via a targeting ligand, including any ligand described herein or known in the art. In preferred embodiments, the targeting ligand is a carbohydrate moiety, e.g., a GalNAc3 ligand, or any other ligand that directs the RNAi agent to a site of interest, e.g., the liver of a subject.

The term “inhibiting,” as used herein, is used interchangeably with “reducing,” “silencing,” “downregulating” and other similar terms, and includes any level of inhibition.

The phrase “inhibiting expression of a Serpina1” is intended to refer to inhibition of expression of any Serpina1 gene (such as, e.g., a mouse Serpina1 gene, a rat Serpina1 gene, a monkey Serpina1 gene, or a human Serpina1 gene) as well as variants or mutants of a Serpina1 gene. Thus, the Serpina1 gene may be a wild-type Serpina1 gene, a mutant Serpina1 gene, or a transgenic Serpina1 gene in the context of a genetically manipulated cell, group of cells, or organism.

“Inhibiting expression of a Serpina1 gene” includes any level of inhibition of a Serpina1 gene, e.g., at least partial suppression of the expression of a Serpina1 gene. The expression of the Serpina1 gene may be assessed based on the level, or the change in the level, of any variable associated with Serpina1 gene expression, e.g., Serpina1 mRNA level, Serpina1 protein level, or lipid levels. This level may be assessed in an individual cell or in a group of cells, including, for example, a sample derived from a subject.

Inhibition may be assessed by a decrease in an absolute or relative level of one or more variables that are associated with Serpina1 expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the invention, expression of a Serpina1 gene is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

Inhibition of the expression of a Serpina1 gene may be manifested by a reduction of the amount of mRNA expressed by a first cell or group of cells (such cells may be present, for example, in a sample derived from a subject) in which a Serpina1 gene is transcribed and which has or have been treated (e.g., by contacting the cell or cells with an RNAi agent of the invention, or by administering an RNAi agent of the invention to a subject in which the cells are or were present) such that the expression of a Serpina1 gene is inhibited, as compared to a second cell or group of cells substantially identical to the first cell or group of cells but which has not or have not been so treated (control cell(s)). In preferred embodiments, the inhibition is assessed by expressing the level of mRNA in treated cells as a percentage of the level of mRNA in control cells, using the following formula:

$$\frac{( mRNA in control cells ) - ( mRNA in treated cells )}{( mRNA in control cells )} \cdot 100 \%$$

Alternatively, inhibition of the expression of a Serpina1 gene may be assessed in terms of a reduction of a parameter that is functionally linked to Serpina1 gene expression, e.g., Serpina1 protein expression, such as ALT, alkaline phosphatase, bilirubin, prothrombin and albumin. Serpina1 gene silencing may be determined in any cell expressing Serpina1, either constitutively or by genomic engineering, and by any assay known in the art. The liver is the major site of Serpina1 expression. Other significant sites of expression include the lung and intestines.

Inhibition of the expression of a Serpina1 protein may be manifested by a reduction in the level of the Serpina1 protein that is expressed by a cell or group of cells (e.g., the level of protein expressed in a sample derived from a subject). As explained above for the assessment of mRNA suppression, the inhibiton of protein expression levels in a treated cell or group of cells may similarly be expressed as a percentage of the level of protein in a control cell or group of cells.

A control cell or group of cells that may be used to assess the inhibition of the expression of a Serpina1 gene includes a cell or group of cells that has not yet been contacted with an RNAi agent of the invention. For example, the control cell or group of cells may be derived from an individual subject (e.g., a human or animal subject) prior to treatment of the subject with an RNAi agent.

The level of Serpina1 mRNA that is expressed by a cell or group of cells may be determined using any method known in the art for assessing mRNA expression. In one embodiment, the level of expression of Serpina1 in a sample is determined by detecting a transcribed polynucleotide, or portion thereof, e.g., mRNA of the Serpina1 gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), Northern blotting, in situ hybridization, and microarray analysis.

In one embodiment, the level of expression of Serpina1 is determined using a nucleic acid probe. The term “probe”, as used herein, refers to any molecule that is capable of selectively binding to a specific Serpina1. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to Serpina1 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of Serpina1 mRNA.

An alternative method for determining the level of expression of Serpina1 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of Serpina1 is determined by quantitative fluorogenic RT-PCR (i.e., the TaqMan™ System).

The expression levels of Serpina1 mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of Serpina1 expression level may also comprise using nucleic acid probes in solution.

In preferred embodiments, the level of mRNA expression is assessed using branched DNA (bDNA) assays or real time PCR (qPCR). The use of these methods is described and exemplified in the Examples presented herein.

The level of Serpina1 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, Western blotting, radioimmunoassay (RIA), enzyme-linked immunosorbent assays (ELISAs), immunofluorescent assays, electrochemiluminescence assays, and the like.

The term “sample” as used herein refers to a collection of similar fluids, cells, or tissues isolated from a subject, as well as fluids, cells, or tissues present within a subject. Examples of biological fluids include blood, serum and serosal fluids, plasma, lymph, urine, cerebrospinal fluid, saliva, ocular fluids, and the like. Tissue samples may include samples from tissues, organs or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In preferred embodiments, a “sample derived from a subject” refers to blood or plasma drawn from the subject. In further embodiments, a “sample derived from a subject” refers to liver tissue derived from the subject.

In some embodiments of the methods of the invention, the RNAi agent is administered to a subject such that the RNAi agent is delivered to a specific site within the subject. The inhibition of expression of Serpina1 may be assessed using measurements of the level or change in the level of Serpina1 mRNA or Serpina1 protein in a sample derived from fluid or tissue from the specific site within the subject. In preferred embodiments, the site is the liver. The site may also be a subsection or subgroup of cells from any one of the aforementioned sites. The site may also include cells that express a particular type of receptor.

### VII. Methods for Treating or Preventing a Serpina1 Associated Disease

The present invention also provides methods for treating or preventing diseases and conditions that can be modulated by down regulating Serpina1 gene expression. For example, the compositions described herein can be used to treat Serpina1 associated diseases, such as liver diseases, e.g., chronic liver disease, liver inflammation, cirrhosis, liver fibrosis, and/or hepatocellular carcinoma, and other pathological conditions that may be associated with these disorders, such as lung inflammation, emphysema, and COPD.

The present invention also provides methods for inhibiting the development of hepatocellular carcinoma in a subject, e.g., a subject having a Serpina1 deficiency variant. The methods include administering a therapeutically effective amount of a composition of the invention to the subject, thereby inhibiting the development of hepatocellular carcinoma in the subject.

Methods and uses of the compositions of the invention for reducing the accumulation of misfolded Serpina1 in the liver of a subject, e.g., a subject having a Serpina1 deficiency variant, are also provided by the present invention. The methods include adminsitering a therapeutically effective amount of a composition of the invention to the subject, thereby reducing the accumulation of misfolded Serpina1 in the liver of the subject.

As used herein, a “subject” includes a human or non-human animal, preferably a vertebrate, and more preferably a mammal. A subject may include a transgenic organism. Most preferably, the subject is a human, such as a human suffering from or predisposed to developing a Serpina1-associated disease. In one embodiment, the subject suffering or predisposed to developing a Serpina1-associated disease has one or more Serpina1 deficient alleles, e.g., a PIZ, PIS, or PIM(Malton) allele.

In further embodiments of the invention, an iRNA agent of the invention is administered in combination with an additional therapeutic agent. The iRNA agent and an additional therapeutic agent can be administered in combination in the same composition, e.g., parenterally, or the additional therapeutic agent can be administered as part of a separate composition or by another method described herein.

Examples of additional therapeutic agents suitable for use in the methods of the invention include those agents known to treat liver disorders, such as liver cirhosis. For example, an iRNA agent featured in the invention can be administered with, e.g., ursodeoxycholic acid (UDCA), immunosuppressive agents, methotrexate, corticosteroids, cyclosporine, colchicine, antipruritic treatments, such as antihistamines, cholestyramine, colestipol, rifampin, dronabinol (Marinol), and plasmaphesesis, prophylactic antibiotics, ultraviolet light, zinc supplements, and hepatitis A, influenza and pneumococci vaccination.

In some embodiments of the methods of the invention, Serpina1 expression is decreased for an extended duration, e.g., at least one week, two weeks, three weeks, or four weeks or longer. For example, in certain instances, expression of the Serpina1 gene is suppressed by at least about 5%, 10%, 15%, 20%, 25%, 30%, 35%, 40%, 45%, 50%, or 55% by administration of an iRNA agent described herein. In some embodiments, the Serpina1 gene is suppressed by at least about 60%, 70%, or 80% by administration of the iRNA agent. In some embodiments, the Serpina1 gene is suppressed by at least about 85%, 90%, or 95% by administration of the iRNA agent.

The iRNA agents of the invention may be administered to a subject using any mode of administration known in the art, including, but not limited to subcutaneous, intravenous, intramuscular, intraocular, intrabronchial, intrapleural, intraperitoneal, intraarterial, lymphatic, cerebrospinal, and any combinations thereof. In preferred embodiments, the iRNA agents are administered subcutaneously.

In some embodiments, the administration is via a depot injection. A depot injection may release the iRNA agents in a consistent way over a prolonged time period. Thus, a depot injection may reduce the frequency of dosing needed to obtain a desired effect, e.g., a desired inhibition of Serpina1, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the RNAi agent to the liver.

Other modes of administration include epidural, intracerebral, intracerebroventricular, nasal administration, intraarterial, intracardiac, intraosseous infusion, intrathecal, and intravitreal, and pulmonary. The mode of administration may be chosen based upon whether local or systemic treatment is desired and based upon the area to be treated. The route and site of administration may be chosen to enhance targeting.

The methods of the invention include administering an iRNA agent at a dose sufficient to suppress/decrease levels of Serpina1 mRNA for at least 5, more preferably 7, 10, 14, 21, 25, 30 or 40 days; and optionally, administering a second single dose of the iRNA agent, wherein the second single dose is administered at least 5, more preferably 7, 10, 14, 21, 25, 30 or 40 days after the first single dose is administered, thereby inhibiting the expression of the Serpina1 gene in a subject.

In one embodiment, doses of an iRNA agent of the invention are administered not more than once every four weeks, not more than once every three weeks, not more than once every two weeks, or not more than once every week. In another embodiment, the administrations can be maintained for one, two, three, or six months, or one year or longer.

In general, the iRNA agent does not activate the immune system, e.g., it does not increase cytokine levels, such as TNF-alpha or IFN-alpha levels. For example, when measured by an assay, such as an in vitro PBMC assay, such as described herein, the increase in levels of TNF-alpha or IFN-alpha, is less than 30%, 20%, or 10% of control cells treated with a control iRNA agent, such as an iRNA agent that does not target Serpina1.

For example, a subject can be administered a therapeutic amount of an iRNA agent, such as 0.5 mg/kg, 1.0 mg/kg, 1.5 mg/kg, 2.0 mg/kg, or 2.5 mg/kg dsRNA. The iRNA agent can be administered by intravenous infusion over a period of time, such as over a 5 minute, 10 minute, 15 minute, 20 minute, or 25 minute period. The administration is repeated, for example, on a regular basis, such as biweekly (i.e., every two weeks) for one month, two months, three months, four months or longer.

After an initial treatment regimen, the treatments can be administered on a less frequent basis. For example, after administration biweekly for three months, administration can be repeated once per month, for six months or a year or longer. Administration of the iRNA agent can reduce Serpina1 levels, e.g., in a cell, tissue, blood, urine, organ (e.g., the liver), or other compartment of the patient by at least 10%, at least 15%, at least 20%, at least 25%, at least 30%, at least 40%, at least 50%, at least 60%, at least 70%, at least 80% or at least 90% or more.

Before administration of a full dose of the iRNA agent, patients can be administered a smaller dose, and monitored for adverse effects, such as an allergic reaction, or for elevated lipid levels or blood pressure. In another example, the patient can be monitored for unwanted immunostimulatory effects, such as increased cytokine (e.g., TNF-alpha or INF-alpha) levels. An exemplary smaller dose is one that results in an incidence of infusion reaction of less than or equal to 5%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of liver fibrosis or amelioration of liver fibrosis can be assessed, for example by periodic monitoring of liver fibrosis markers: a-2-macroglobulin(a-MA), transferrin, apolipoproteinAl, hyaluronic acid (HA), laminin, N-terminal procollagen III(PIIINP), 7S collagen IV (7S-IV), total bilirubin, indirect bilirubin, alanine aminotransferase (ALT), aspartate aminotransferase(AST), AST/ALT, g-glutamyl transpeptidase(GGT), alkaline phosphatase(ALP), albumin, albumin/globulin, blood urea nitrogen(BUN), creatinine(Cr), triglyceride, cholersterol, high density lipoprotein and low density lipoprotein and liver puncture biopsy. Liver fibrosis markers can be measured and/or liver puncture biopsy can be performed before treatment (initial readings) and subsequently (later readings) during the treatment regimen.

Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an iRNA agent targeting Serpina1 or pharmaceutical composition thereof, “effective against” a Serpina1 associate disease, such as a liver disease, e.g., a hepatic fibrosis condition, indicates that administration of an iRNA agent of the invention in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating liver diseases.

In the methods of the invention, an iRNA agent as described herein can be used to treat individuals having the signs, symptoms and/or markers of, or being diagnosed with, or being a risk of having an Serpina1 associate disease, such as a liver disease, e.g., liver inflammation, cirrhosis, liver fibrosis, and/or hepatoceullar carcinoma. One of skill in the art can easily monitor the signs, symptoms, and/or makers of such disorders in subjects receiving treatment with an iRNA agent as described herein and assay for a reduction in these signs, symptoms and/or makers of at least 10% and preferably to a clinical level representing a low risk of liver disease.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease (such as a liver function described supra), and preferably at least 20%, 30%, 40%, 50% or more can be indicative of effective treatment.

Efficacy for a given iRNA agent of the invention or formulation of that iRNA agent can also be judged using an experimental animal model for the given disease as known in the art. When using an experimental animal model, efficacy of treatment is evidenced when a statistically significant reduction in a marker or symptom is observed.

A treatment or preventive effect is also evident when one or more symtoms are reduced or alleveiated. For example, a treatment or preventive is effective when one or more of weakness, fatigue, weight loss, nausea, vomiting, abdominal swelling, extremity swelling, excessive itching, and jaundice of the eyes and/or skin is reduced or alleviated.

For certain indications, the efficacy can be measured by an increase in serum levels of Serpina1 protein. As an example, an increase of serum levels of properly folded Serpina1 of at least 10%, at least 20%, at least 50%, at least 100%, at least 200% more can be indicative of effective treatment.

Alternatively, the efficacy can be measured by a reduction in the severity of disease as determined by one skilled in the art of diagnosis based on a clinically accepted disease severity grading scale, as but one example the Child-Pugh score (sometimes the Child-Turcotte-Pugh score). In this example, prognosis of chronic liver disease, mainly cirrhosis, is measured by an aggregate score of five clinical measures, billirubin, serum albumin, INR, ascites, and hepatic encephalopathy. Each marker is assigned a value from 1-3, and the total value is used to provide a score categorized as A (5-6 points), B (7-9 points), or C (10-15 points), which can be correlated with one and two year survival rates. Methods for determination and analysis of Child-Pugh scores are well known in the art (Farnsworth et al, Am J Surgery 2004 188:580-583; Child and Turcotte. Surgery and portal hypertension. In: The liver and portal hypertension. Edited by CG Child. Philadelphia: Saunders 1964:50-64; Pugh et al, Br J Surg 1973; 60:648-52). Efficacy can be measured in this example by the movement of a patient from e.g., a “B” to an “A.” Any positive change resulting in e.g., lessening of severity of disease measured using the appropriate scale, represents adequate treatment using an iRNA or iRNA formulation as described herein.

In one embodiment, the RNAi agent is administered at a dose of between about 0.25 mg/kg to about 50 mg/kg, e.g., between about 0.25 mg/kg to about 0.5 mg/kg, between about 0.25 mg/kg to about 1 mg/kg, between about 0.25 mg/kg to about 5 mg/kg, between about 0.25 mg/kg to about 10 mg/kg, between about 1 mg/kg to about 10 mg/kg, between about 5 mg/kg to about 15 mg/kg, between about 10 mg/kg to about 20 mg/kg, between about 15 mg/kg to about 25 mg/kg, between about 20 mg/kg to about 30 mg/kg, between about 25 mg/kg to about 35 mg/kg, or between about 40 mg/kg to about 50 mg/kg.

The dose of an RNAi agent that is administered to a subject may be tailored to balance the risks and benefits of a particular dose, for example, to achieve a desired level of Serpina1 gene suppression (as assessed, e.g., based on Serpina1 mRNA suppression, Serpina1 protein expression) or a desired therapeutic or prophylactic effect, while at the same time avoiding undesirable side effects.

In some embodiments, the RNAi agent is administered in two or more doses. If desired to facilitate repeated or frequent infusions, implantation of a delivery device, e.g., a pump, semi-permanent stent (e.g., intravenous, intraperitoneal, intracisternal or intracapsular), or reservoir may be advisable. In some embodiments, the number or amount of subsequent doses is dependent on the achievement of a desired effect, e.g., the suppression of a Serpina1 gene, or the achievement of a therapeutic or prophylactic effect, e.g., reducing reducing a symptom of a liver disease. In some embodiments, the RNAi agent is administered according to a schedule. For example, the RNAi agent may be administered once per week, twice per week, three times per week, four times per week, or five times per week. In some embodiments, the schedule involves regularly spaced administrations, e.g., hourly, every four hours, every six hours, every eight hours, every twelve hours, daily, every 2 days, every 3 days, every 4 days, every 5 days, weekly, biweekly, or monthly. In other embodiments, the schedule involves closely spaced administrations followed by a longer period of time during which the agent is not administered. In some embodiments, the RNAi agent is administered in a dosing regimen that includes a “loading phase” of closely spaced administrations that may be followed by a “maintenance phase”, in which the RNAi agent is administred at longer spaced intervals.

Any of these schedules may optionally be repeated for one or more iterations. The number of iterations may depend on the achievement of a desired effect, e.g., the suppression of a Serpina1 gene, and/or the achievement of a therapeutic or prophylactic effect, e.g., reducing a symptom of a Serpina1 associated disease, e.g., a liver disease.

In another aspect, the invention features, a method of instructing an end user, e.g., a caregiver or a subject, on how to administer an iRNA agent described herein. The method includes, optionally, providing the end user with one or more doses of the iRNA agent, and instructing the end user to administer the iRNA agent on a regimen described herein, thereby instructing the end user.

Genetic predisposition plays a role in the development of target gene associated diseases, e.g., liver disease. Therefore, a patient in need of a siRNA can be identified by taking a family history, or, for example, screening for one or more genetic markers or variants. Accordingly, in one aspect, the invention provides a method of treating a patient by selecting a patient on the basis that the patient has one or more of a Serpina1 deficiency or a Serpina1 deficiency gene variant, e.g., a PIZ, PIS, or PIM(Malton) allele. The method includes administering to the patient an iRNA agent in a therapeutically effective amount.

A healthcare provider, such as a doctor, nurse, or family member, can take a family history before prescribing or administering an iRNA agent of the invention. In addition, a test may be performed to determine a geneotype or phenotype. For example, a DNA test may be performed on a sample from the patient, e.g., a blood sample, to identify the Serpina1 genotype and/or phenotype before a Serpina1 dsRNA is administered to the patient.

### VIII. Kits

The present invention also provides kits for using any of the iRNA agents and/or performing any of the methods of the invention. Such kits include one or more RNAi agent(s) and instructions for use, e.g., instructions for inhibiting expression of a Serpina1 in a cell by contacting the cell with the RNAi agent(s) in an amount effective to inhibit expression of the Serpina1. The kits may optionally further comprise means for contacting the cell with the RNAi agent (e.g., an injection device), or means for measuring the inhibition of Serpina1 (e.g., means for measuring the inhibition of Serpina1 mRNA). Such means for measuring the inhibition of Serpina1 may comprise a means for obtaining a sample from a subject, such as, e.g., a plasma sample. The kits of the invention may optionally further comprise means for administering the RNAi agent(s) to a subject or means for determining the therapeutically effective or prophylactically effective amount.

Unless otherwise defined, all technical and scientific terms used herein have the same meaning as commonly understood by one of ordinary skill in the art to which this invention belongs. Although methods and materials similar or equivalent to those described herein can be used in the practice or testing of the iRNAs and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. Mitigation of Off-Target Effects and In Vivo Toxicity

RNA interference or “RNAi” is a term initially coined by Fire and co-workers to describe the observation that double stranded RNAi (dsRNA) can block gene expression (Fire et al. (1998) Nature 391, 806-811; Elbashir et al. (2001) Genes Dev. 15, 188-200). Short dsRNA directs gene-specific, post-transcriptional silencing in many organisms, including vertebrates, and has provided a new tool for studying gene function. RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double stranded RNA trigger, but the protein components of this activity remained unknown.

One of the off-target effects of siRNA is the miRNA-like effect—the argonaute protein, the core effector in RNA interference, treats siRNA, which is artificially introduced in order to induce RNA interference, as a miRNA (microRNA). (Lam et al. (2015) Molecular Therapy Nucleic Acids (2015) 4, e252). The miRNA recognizes a target gene majorly through base-pairing between the seed region (positions 2-7 from the 5′ end) and the target mRNA for gene suppression. The off-targets caused by siRNAs originate from base-complemtarity of the seed regions of the RISC-loaded antisense strand of siRNA with one or more mRNA. The miRNA-like off-target effects in siRNAs have been reported in several studies, and affect expression of multitude of genes depending on sequences of the seed regions and are serious enough to cause up to 30% of the positive hits in siRNA based phenotype screening. Additionally, in the case of miRNAs, they are also reported to silence target genes through compensatory pairings within their 3′ end regions (3′-compensatory pairing) when the interactions between seed region and targets become weak, implicating that the miRNA-like off-target effects are likely to be mediated by such mechanism.

As described below, it has been discovered that dsRNA agents where the antisense strand comprises at least one thermally destabilizing modification of the duplex within the seed region (i.e., at position 2-9 of the 5′-end of the antisense strand, counting from the 5′-end) and/or the dsRNA agent has a melting temperature in the range of from about 40° C. to about 80° C. can be more effective in mediating RNA interference than a parent dsRNA agent lacking the destabilizing modification. Such agents are advantageous for inhibition of target gene expression, while having reduced off-target gene silencing effects, as well as RNAi compositions suitable for therapeutic use.

### Materials and Methods

The following materials and methods were used in the Examples.

siRNA Design

Lead development candidate dsRNA agents targeting serine peptidase inhibitor, clade A, member 1 (Serpina1) (AAT) were selected for evaluation in the experiments described herein. dsRNA agents targeting Serpina1 having the modified nucleotide sequence of the parent lead development candidate and having an antisense strand comprising at least one thermally destabilizing modification of the duplex within the seed region (i.e., at position 2-9 of the 5′-end of the antisense strand, counting from the 5′-end) and/or having a melting temperature in the range of from about 40° C. to about 80° C. were designed, synthesized and evaluated as described below.

Specifically, as described in U.S. Provisional Application No. 61/826,125, filed on May 22, 2013, U.S. Provisional Application No. 61/898,695, filed on Nov. 1, 2013; U.S. Provisional Application No. 61/979,727, filed on Apr. 15, 2014; U.S. Provisional Application No. 61/989,028, filed on May 6, 2014; U.S. patent application Ser. No. 14/284,745, now issued as U.S. Pat. No. 9,574,192, issued on Feb. 21, 2017; U.S. patent application Ser. No. 15/399,820, filed on Jan. 6, 2017; and International Patent Application No. PCT/US2014/039109, filed on May 22, 2014, AD-61444 was identified and shown to effectively and durably inhibit AAT in vitro and in vivo. The unmodified sense strand nucleotide sequence of AD-61444 is 5′-CUUCUUAAUGAUUGAACAAAA-3′ (SEQ ID NO: 417) and the unmodified nucleotide sequence of AD-61444 is 5′-UUUUGUUCAAUCAUUAAGAAGAC-3′ (SEQ ID NO: 419). The modified sense strand nucleotide sequence of AD-61444 is 5′-csusucuuaauGfAfuugaacaaaaL96-3′ (SEQ ID NO: 418) and the modified antisense strand nucleotide sequence of AD-61444 is 5′-usUfsuUfgUfuCfaAfucaUfuAfaGfaAfgsasc-3′ (SEQ ID NO: 420).

TABLE B
Table 1: Abbreviations of nucleotide monomers used in nucleic
acid sequence representation.
It will be understood that these monomers, when present in an
oligonucleotide, are mutually linked by 5′-3′-phosphodiester bonds.

| Abbreviation | Nucleotide(s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′ -fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′ -fluorouridine -3′-phosphorothioate |
| Us | uridine -3′-phosphorothioate |
| N | any nucleotide (G, A, C, T or U) |
| a | 2′-O-methyladenosine-3′-phosphate |
| as | 2′-O-methyladenosine-3′ - phosphorothioate |
| C | 2′-O-methylcytidine-3′-phosphate |
| CS | 2′-O-methylcytidine-3′- phosphorothioate |
| g | 2′-O-methylguanosine-3′-phosphate |
| gs | 2′-O-methylguanosine-3′ - phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| S | phosphorothioate linkage |
| L96 | N-[tris(GalNAc-alkyl)-amidodecanoyl)]-4-hydroxyprolinol |
| | Hyp-(GalNAc-alkyl)3 |
| Y44 | inverted abasic DNA (2-hydroxymethyl-tetrahydrofurane-5- |
| | phosphate) |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | Vinyl-phosphate |
| (Aam) | 2′-O-(N-methylacetamide)adenosine-3′-phosphate |
| (Aams) | 2′-O-(N-methylacetamide)adenosine-3′-phosphorothioate |
| (Tam) | 2′-O-(N-methylacetamide)thymidine-3′-phosphate |
| (Tams) | 2′-O-(N-methylacetamide)thymidine-3′-phosphorothioate |
| dA | 2′-deoxyadenosine-3′-phosphate |
| dAs | 2′ -deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |

### Synthesis and Purification

All oligonucleotides were prepared on a MerMade 192 synthesizer on a 1 μmole scale using universal or custom supports. All phosphoramidites were used at a concentration 100 mM in 100% Acetonitrile or 9:1 Acetonitrile:DMF with a standard protocol for 2-cyanoethyl phosphoramidites, except that the coupling time was extended to 400 seconds. Oxidation of the newly formed linkages was achieved using a solution of 50 mM 12 in 9:1 Acetonitrile:Water to create phosphate linkages and 100 mM DDTT in 9:1 Pyridine:Acetonitrile to create phosphorothioate linkages. After the trityl-off synthesis, columns were incubated with 150 μL of 40% aqueous Methylamine for 45 minutes and the solution drained via vacuum into a 96-well plate. After repeating the incubation and draining with a fresh portion of aqueous Methylamine, the plate containing crude oligonucleotide solution was sealed and shaken at room temperature for an additional 60 minutes to completely remove all protecting groups. of the crude oligonucleotides was accomplished via the addition of 1.2 mL of 9:1 Acetonitrile:EtOH to each well followed by incubation at −20° C. overnight. The plate was then centrifuged at 3000 RPM for 45 minutes, the supernatant removed from each well, and the pellets resuspended in 950 μL of 20 mM aqueous NaOAc. Each crude solution was finally desalted over a GE Hi-Trap Desalting Column (Sephadex G25 Superfine) using water to elute the final oligonucleotide products. All identities and purities were confirmed using ESI-MS and IEX HPLC, respectively.

### Temperature-Dependent UV Spectroscopy

The melting studies were performed at a duplex concentration of 1 μM (consisting of the modified antisense strand paired with the complementary unmodified RNA sense strand) in 0.33×PBS (3.3 mM Na/K phosphate buffer, pH 7.4, with 46 mM NaCl and 0.9 mM KCl) in 1 cm path length quartz cells on a Beckman DU800 spectrophotometer equipped with a thermoprogrammer. Each cuvette contained 200 μL of sample solution covered by 125 μL of light mineral oil. Melting curves were monitored at 260 nm with a heating rate of 1° C./min from 15-90° C. Melting temperatures (Tm) were calculated from the first derivatives of the smoothed heating curves and the reported values are the result of at least two independent measurements.

### In Vitro Reporter Assays

COS-7 cells were cultured at 37° C., 5% CO2 in Dulbecco's Modified Eagle Medium (DMEM) supplemented with 10% fetal bovine serum (FBS). Cells were co-transfected in 96-well plates (15,000 cells/well) with 10 ng luciferase reporter plasmid and 50 fM to 50 nM siRNA in 10-fold dilutions using 2 μg/mL Lipofectamine 2000 (Thermo Fisher Scientific) according to manufacturer's instructions. Cells were harvested at 48 h after transfection for the dual luciferase assay (Promega) according to manufacturer's instructions. The on-target reporter plasmid contained a single perfectly-complementary site to the antisense strand in the 3′ untranslated (3′ UTR) of Renilla luciferase. The off-target reporter plasmid contained four tandem seed-complementary sites separated by 21-28 nucleotides in the 3′ UTR of Renilla luciferase. Both plasmids co-expressed Firefly luciferase as a transfection control.

### Gene Expression Analysis

Cryopreserved rat and human hepatocytes (Bioreclamation) were cultured at 37° C., 5% CO2 in InVitroGRO CP Medium with Torpedo Antibiotic Mix. Cells were transfected in 96-well plates (20,000 cells/well) with 10 nM siRNA using 2 μg/mL Lipofectamine RNAiMAX (Thermo Fisher Scientific) according to manufacturer's instructions. Cells were harvested at 24 h after transfection for RNA extraction with the miRNeasy Kit (Qiagen) according to manufacturer's instructions. cDNA libraries were prepared using the TruSeq Stranded Total RNA Library Prep Kit (Illumina) and sequenced on NextSeq 500 (Illumina).

### In Vivo Mouse and Rat Studies

All studies were conducted using protocols consistent with local, state and federal regulations as applicable and approved by the Institutional Animal Care and Use Committees (IACUCs) at Alnylam Pharmaceuticals.

In mouse pharmacodynamic studies, female C57BL/6 mice (Charles River Laboratories) were administered a single dose of a vehicle control (0.9% sodium chloride, saline) or 0.5 or 1 mg/kg siRNA subcutaneously in the upper back. On Day 8, livers were collected, rinsed in cold saline, immediately snap frozen in liquid nitrogen, and stored at −80 C for mRNA and siRNA analysis.

In rat toxicity studies, male Sprague Dawley rats (Charles River Laboratories) were administered three repeat weekly doses (qw×3) of a vehicle control (0.9% sodium chloride, saline) or 30 mg/kg siRNA subcutaneously in the upper back. On Day 16, serum was collected for clinical pathology evaluation, and livers were collected for histopathology evaluation and for mRNA and siRNA analysis.

mRNA and siRNA Quantitation

RNA was extracted with the miRNeasy Kit (Qiagen) according to manufacturer's instructions, converted to cDNA with the High-Capacity cDNA Reverse Transcription Kit (Thermo Fisher Scientific) according to manufacturer's instructions, and mRNA levels were assessed by quantitative polymerase chain reaction (qPCR) using gene-specific Taqman probes (Thermo Fisher Scientific) on Roche Light Cycler 480 II using LightCycler 480 Probes Master (Roche).

To quantitate exposure to siRNAs, cell pellets were resuspended in phosphate-buffer saline (PBS) containing 0.25% Triton X-100, heated at 95° C. for 10 min, centrifuged at 14,000 rpm at 4° C. for 10 min, and reverse transcription was performed on the supernatants using TaqMan MicroRNA Reverse Transcription Kit (Thermo Fisher Scientific) according to the manufacturer's instructions. qPCR was performed on Roche Light Cycler 480 II using LightCycler 480 Probes Master (Roche) according to the manufacturer's instructions.

### Results

### 1. In Vitro Studies

Results of in vitro reporter assay are summarized in Table 1. As the data in Table 1 show, glycolic nucleic acid (GNA) modification at position 7 of the antisense strand preserves the on-target activity while mitigating the off-target activity in vitro.

TABLE 1
In vitro reporter assays data for GNA modification
at position 7 of anti-sense strands

| | | On-target IC₅₀ | Off-target IC₅₀ |
|---|---|---|---|
| | | (nM) | (nM) |
| AAT | Parent | 0.013 | 0.97 |
| | (S)-GNA at AS pos. 7 | 0.013 | >500 |

Luciferase reporter plasmids were co-transfected with siRNAs into COS-7 cells and the luciferase assay was performed at 48 h.

### 2. Gene Expression Analysis

Results of in vitro gene expression analysis are summarized in Table 2. As seen from Table 2, GNA modification at position 7 of the antisense strand mitigated off-target activity in vitro.

TABLE 2
Gene expression analysis

| | | Number of down- | Number of up- |
|---|---|---|---|
| | | regulated genes | regulated genes |
| | | (p < 0.05) | (p < 0.05) |
| AAT | Parent | 100 | 11 |
| | (S)-GNA at AS pos. 7 | 1 | 2 |

Human hepatocytes (AAT) were transfected with siRNAs and RNA was collected at 24 h for RNA sequencing.

### 3. In Vivo Mouse Studies

Results of in vivo studies are summarized in Table 3. As seen, GNA modification at position 7 of the antisense strand preserves potency in vivo (Table 3).

TABLE 3
Mouse pharmacodynamics data

| | | % mRNA remaining at Day 8 |
|---|---|---|
| AAT | Parent | 30 |
| | (S)-GNA at AS pos. 7 | 29 |

Mice were administered a single dose of siRNAs targeting AAT at 1 mg/kg and liver mRNA knockdown was assessed at Day 8.

### Example 2. In Vivo and In Vitro Analysis of dsRNA Agents Targeting Serpina1

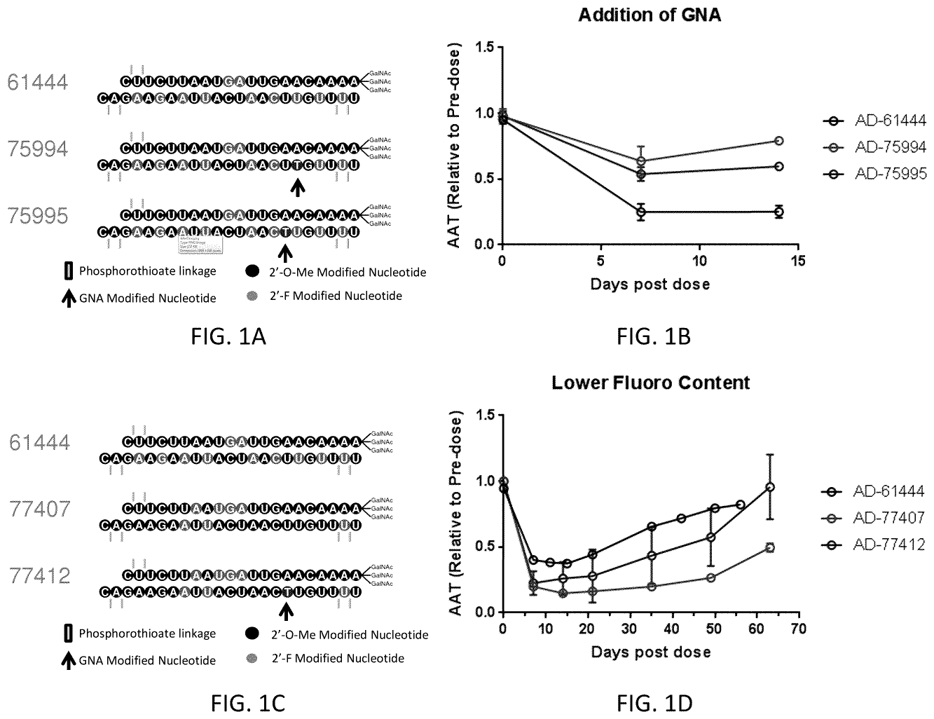

To further evaluate the in vivo effect of incorporating a GNA nucleotide in AD-61444, AD-61444 was further modified with a GNA nucleotide at antisense position 6 (AD-75994) or 7 (AD-75995) (FIG. 1A) and mice transgenic for human AAT were subcutaneously administered a single1 mg/kg dose of the agent. Prior to dosing, and at the timepoints indicated in FIG. 1B, serum was obtained for quantification of circulating AAT. AAT protein levels were determined by sandwich ELISA, and the data was normalized to the baseline value of AAT protein levels.

As depicted in FIG. 1B, incorporation of a GNA nucleotide at either position 6 or 7 of the antisense strand of AD-61444 resulted in a loss of activity as compared to the parent molecule.

Accordingly, the total numer of 2′-fluoro modified nucleotides in AD-61444 was reduced resulting in AD-77407, and a GNA nucleotide was incorporated at antisense postion 7 of AD-77407 resulting in AD-77412 (FIG. 1C). Mice transgenic for human AAT were subcutaneously administered a single 1 mg/kg dose of the agent. Prior to dosing, and at the timepoints indicated in FIG. 1D, serum was obtained for quantification of circulating AAT. AAT protein levels were determined by sandwich ELISA, and the data was normalized to the baseline value of AAT protein levels.

FIG. 1D demonstrates that reducing the 2′ flouro content of AD-61444 (i.e., AD-77407) resulted in a marked improvement in activity in the mice. Moreover, incorporation of a GNA nucleotide in this context (AD-77412) resulted in a net improvement relative to the parent molecule, AD-61444.

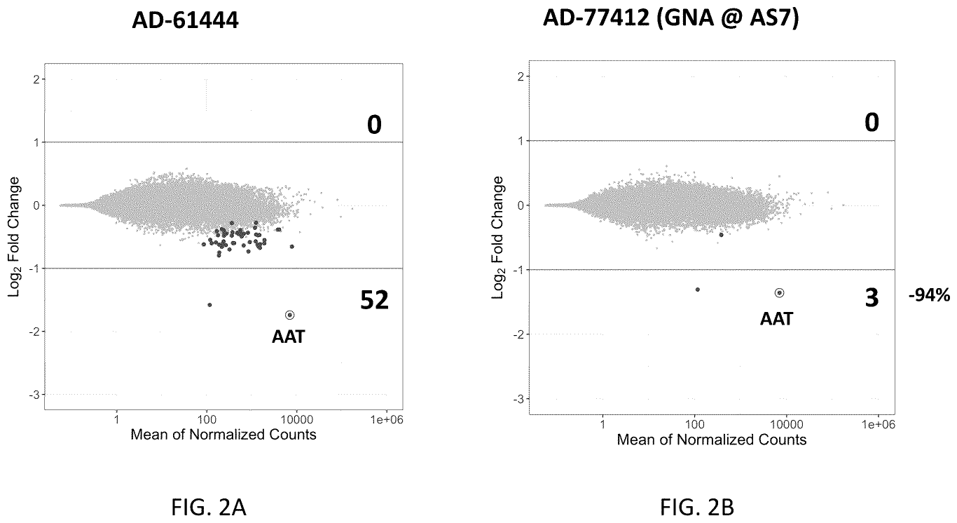

To determine the effect of reducing the 2′-fluoro content of AD-61444 and incorporation of a GNA nucleotide at position 7 of the antisense strand on off-target effects, Hep3b cells were transfected with 10 nM of AD-61444 or AD-77412 using Lipofectamine RNAiMax. After 16 hours, cells were lysed and prepared for transcriptional profiling. As depicted in FIGS. 2A and 2B, the resulting data was graphed showing transcripts whose expression was statistically significantly different relative to mock treated cells as black circles and transcripts whose expression was not statistically significantly different relative to mock treated cells as gray circles. The AAT transcript is indicated and highlighted with a circle.

The data depicted in FIGS. 2A and 2B demonstrate that treatment of cells with AD-61444 resulted in downregulation of fifty-two transcripts, including AAT. By comparison, the GNA-containing duplex, AD-77412, resulted in the downregulated transcripts of only three transcripts, including AAT. Thus, incorporation of a GNA nucleotide substantially reduced the number of observed off-targets downregulated by AD-61444 in vitro.

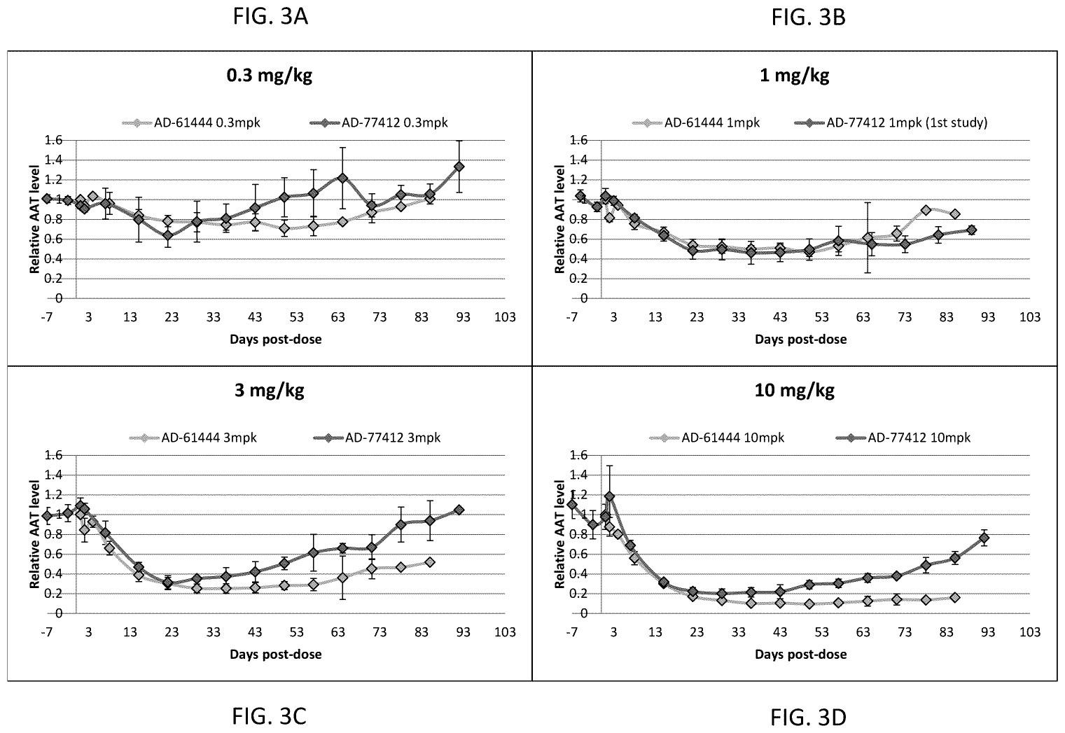

The effect of AD-77412 was also evaluated in non-human primates. Cynomolgus monkeys were subcutaeoulsy administered a single 0.3 mg/kg, 1.0 mg/kg, 3 mg/kg, or 10 mg/kg dose of either AD-61444 or AD-77412. Prior to dosing, and at the timepoints indicated in FIGS. 3A-3D, serum was obtained for quantification of circulating AAT. AAT protein levels were determined by sandwich ELISA, and the data was normalized to the baseline value of AAT protein levels.

As depicted in FIGS. 3A-3D, all doses of AD-61444 and AD-77412 effectively silenced AAT expression with similar maximum lavels of silencing.

In summary, these assays demonstrate that incorporation of a GNA nucleotide into the antisense seed region of the parent molecule, AD-61444, does not result in loss of activity as compared to the parent molecule and significantly reduces off-target effects.

| Informal Sequence Listing | |
|---|---|
| <210> 1 | |
| <211> 3220 | |
| <212> DNA | |
| <213> Homo sapiens | |
| <400> 1 | |
| acaatgactc ctttcggtaa gtgcagtgga agctgtacac tgcccaggca aagcgtccgg | 60 |
| gcagcgtagg cgggcgactc agatcccagc cagtggactt agcccctgtt tgctcctccg | 120 |
| ataactgggg tgaccttggt taatattcac cagcagcctc ccccgttgcc cctctggatc | 180 |
| cactgcttaa atacggacga ggacagggcc ctgtctcctc agcttcaggc accaccactg | 240 |
| acctgggaca gtgaatcgac aatgccgtct tctgtctcgt ggggcatcct cctgctggca | 300 |
| ggcctgtgct gcctggtccc tgtctccctg gctgaggatc cccagggaga tgctgcccag | 360 |
| aagacagata catcccacca tgatcaggat cacccaacct tcaacaagat cacccccaac | 420 |
| ctggctgagt tcgccttcag cctataccgc cagctggcac accagtccaa cagcaccaat | 480 |
| atcttcttct ccccagtgag catcgctaca gcctttgcaa tgctctccct ggggaccaag | 540 |
| gctgacactc acgatgaaat cctggagggc ctgaatttca acctcacgga gattccggag | 600 |
| gctcagatcc atgaaggctt ccaggaactc ctccgtaccc tcaaccagcc agacagccag | 660 |
| ctccagctga ccaccggcaa tggcctgttc ctcagcgagg gcctgaagct agtggataag | 720 |
| tttttggagg atgttaaaaa gttgtaccac tcagaagcct tcactgtcaa cttcggggac | 780 |
| accgaagagg ccaagaaaca gatcaacgat tacgtggaga agggtactca agggaaaatt | 840 |
| gtggatttgg tcaaggagct tgacagagac acagtttttg ctctggtgaa ttacatcttc | 900 |
| tttaaaggca aatgggagag accctttgaa gtcaaggaca ccgaggaaga ggacttccac | 960 |
| gtggaccagg tgaccaccgt gaaggtgcct atgatgaagc gtttaggcat gtttaacatc | 1020 |
| cagcactgta agaagctgtc cagctgggtg ctgctgatga aatacctggg caatgccacc | 1080 |
| gccatcttct tcctgcctga tgaggggaaa ctacagcacc tggaaaatga actcacccac | 1140 |
| gatatcatca ccaagttcct ggaaaatgaa gacagaaggt ctgccagctt acatttaccc | 1200 |
| aaactgtcca ttactggaac ctatgatctg aagagcgtcc tgggtcaact gggcatcact | 1260 |
| aaggtcttca gcaatggggc tgacctctcc ggggtcacag aggaggcacc cctgaagctc | 1320 |
| tccaaggccg tgcataaggc tgtgctgacc atcgacgaga aagggactga agctgctggg | 1380 |
| gccatgtttt tagaggccat acccatgtct atcccccccg aggtcaagtt caacaaaccc | 1440 |
| tttgtcttct taatgattga acaaaatacc aagtctcccc tcttcatggg aaaagtggtg | 1500 |
| aatcccaccc aaaaataact gcctctcgct cctcaacccc tcccctccat ccctggcccc | 1560 |
| ctccctggat gacattaaag aagggttgag ctggtccctg cctgcatgtg actgtaaatc | 1620 |
| cctcccatgt tttctctgag tctccctttg cctgctgagg ctgtatgtgg gctccaggta | 1680 |
| acagtgctgt cttcgggccc cctgaactgt gttcatggag catctggctg ggtaggcaca | 1740 |
| tgctgggctt gaatccaggg gggactgaat cctcagctta cggacctggg cccatctgtt | 1800 |
| tctggagggc tccagtcttc cttgtcctgt cttggagtcc ccaagaagga atcacagggg | 1860 |
| aggaaccaga taccagccat gaccccaggc tccaccaagc atcttcatgt ccccctgctc | 1920 |
| atcccccact cccccccacc cagagttgct catcctgcca gggctggctg tgcccacccc | 1980 |
| aaggctgccc tcctgggggc cccagaactg cctgatcgtg ccgtggccca gttttgtggc | 2040 |
| atctgcagca acacaagaga gaggacaatg tcctcctctt gacccgctgt cacctaacca | 2100 |
| gactcgggcc ctgcacctct caggcacttc tggaaaatga ctgaggcaga ttcttcctga | 2160 |
| agcccattct ccatggggca acaaggacac ctattctgtc cttgtccttc catcgctgcc | 2220 |
| ccagaaagcc tcacatatct ccgtttagaa tcaggtccct tctccccaga tgaagaggag | 2280 |
| ggtctctgct ttgttttctc tatctcctcc tcagacttga ccaggcccag caggccccag | 2340 |
| aagaccatta ccctatatcc cttctcctcc ctagtcacat ggccataggc ctgctgatgg | 2400 |
| ctcaggaagg ccattgcaag gactcctcag ctatgggaga ggaagcacat cacccattga | 2460 |
| cccccgcaac ccctcccttt cctcctctga gtcccgactg gggccacatg cagcctgact | 2520 |
| tctttgtgcc tgttgctgtc cctgcagtct tcagagggcc accgcagctc cagtgccacg | 2580 |
| gcaggaggct gttcctgaat agcccctgtg gtaagggcca ggagagtcct tccatcctcc | 2640 |
| aaggccctgc taaaggacac agcagccagg aagtcccctg ggcccctagc tgaaggacag | 2700 |
| cctgctccct ccgtctctac caggaatggc cttgtcctat ggaaggcact gccccatccc | 2760 |
| aaactaatct aggaatcact gtctaaccac tcactgtcat gaatgtgtac ttaaaggatg | 2820 |
| aggttgagtc ataccaaata gtgatttcga tagttcaaaa tggtgaaatt agcaattcta | 2880 |
| catgattcag tctaatcaat ggataccgac tgtttcccac acaagtctcc tgttctctta | 2940 |
| agcttactca ctgacagcct ttcactctcc acaaatacat taaagatatg gccatcacca | 3000 |
| agccccctag gatgacacca gacctgagag tctgaagacc tggatccaag ttctgacttt | 3060 |
| tccccctgac agctgtgtga ccttcgtgaa gtcgccaaac ctctctgagc cccagtcatt | 3120 |
| gctagtaaga cctgcctttg agttggtatg atgttcaagt tagataacaa aatgtttata | 3180 |
| cccattagaa cagagaataa atagaactac atttcttgca | 3220 |
| <210> 2 | |
| <211> 3199 | |
| <212> DNA | |
| <213> Homo sapiens | |
| <400> 2 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaaggaca | 240 |
| atgccgtctt ctgtctcgtg gggcatcctc ctgctggcag gcctgtgctg cctggtccct | 300 |
| gtctccctgg ctgaggatcc ccagggagat gctgcccaga agacagatac atcccaccat | 360 |
| gatcaggatc acccaacctt caacaagatc acccccaacc tggctgagtt cgccttcagc | 420 |
| ctataccgcc agctggcaca ccagtccaac agcaccaata tcttcttctc cccagtgagc | 480 |
| atcgctacag cctttgcaat gctctccctg gggaccaagg ctgacactca cgatgaaatc | 540 |
| ctggagggcc tgaatttcaa cctcacggag attccggagg ctcagatcca tgaaggcttc | 600 |
| caggaactcc tccgtaccct caaccagcca gacagccagc tccagctgac caccggcaat | 660 |
| ggcctgttcc tcagcgaggg cctgaagcta gtggataagt ttttggagga tgttaaaaag | 720 |
| ttgtaccact cagaagcctt cactgtcaac ttcggggaca ccgaagaggc caagaaacag | 780 |
| atcaacgatt acgtggagaa gggtactcaa gggaaaattg tggatttggt caaggagctt | 840 |
| gacagagaca cagtttttgc tctggtgaat tacatcttct ttaaaggcaa atgggagaga | 900 |
| ccctttgaag tcaaggacac cgaggaagag gacttccacg tggaccaggt gaccaccgtg | 960 |
| aaggtgccta tgatgaagcg tttaggcatg tttaacatcc agcactgtaa gaagctgtcc | 1020 |
| agctgggtgc tgctgatgaa atacctgggc aatgccaccg ccatcttctt cctgcctgat | 1080 |
| gaggggaaac tacagcacct ggaaaatgaa ctcacccacg atatcatcac caagttcctg | 1140 |
| gaaaatgaag acagaaggtc tgccagctta catttaccca aactgtccat tactggaacc | 1200 |
| tatgatctga agagcgtcct gggtcaactg ggcatcacta aggtcttcag caatggggct | 1260 |
| gacctctccg gggtcacaga ggaggcaccc ctgaagctct ccaaggccgt gcataaggct | 1320 |
| gtgctgacca tcgacgagaa agggactgaa gctgctgggg ccatgttttt agaggccata | 1380 |
| cccatgtcta tcccccccga ggtcaagttc aacaaaccct ttgtcttctt aatgattgaa | 1440 |
| caaaatacca agtctcccct cttcatggga aaagtggtga atcccaccca aaaataactg | 1500 |
| cctctcgctc ctcaacccct cccctccatc cctggccccc tccctggatg acattaaaga | 1560 |
| agggttgagc tggtccctgc ctgcatgtga ctgtaaatcc ctcccatgtt ttctctgagt | 1620 |
| ctccctttgc ctgctgaggc tgtatgtggg ctccaggtaa cagtgctgtc ttcgggcccc | 1680 |
| ctgaactgtg ttcatggagc atctggctgg gtaggcacat gctgggcttg aatccagggg | 1740 |
| ggactgaatc ctcagcttac ggacctgggc ccatctgttt ctggagggct ccagtcttcc | 1800 |
| ttgtcctgtc ttggagtccc caagaaggaa tcacagggga ggaaccagat accagccatg | 1860 |
| accccaggct ccaccaagca tcttcatgtc cccctgctca tcccccactc ccccccaccc | 1920 |
| agagttgctc atcctgccag ggctggctgt gcccacccca aggctgccct cctgggggcc | 1980 |
| ccagaactgc ctgatcgtgc cgtggcccag ttttgtggca tctgcagcaa cacaagagag | 2040 |
| aggacaatgt cctcctcttg acccgctgtc acctaaccag actcgggccc tgcacctctc | 2100 |
| aggcacttct ggaaaatgac tgaggcagat tcttcctgaa gcccattctc catggggcaa | 2160 |
| caaggacacc tattctgtcc ttgtccttcc atcgctgccc cagaaagcct cacatatctc | 2220 |
| cgtttagaat caggtccctt ctccccagat gaagaggagg gtctctgctt tgttttctct | 2280 |
| atctcctcct cagacttgac caggcccagc aggccccaga agaccattac cctatatccc | 2340 |
| ttctcctccc tagtcacatg gccataggcc tgctgatggc tcaggaaggc cattgcaagg | 2400 |
| actcctcagc tatgggagag gaagcacatc acccattgac ccccgcaacc cctccctttc | 2460 |
| ctcctctgag tcccgactgg ggccacatgc agcctgactt ctttgtgcct gttgctgtcc | 2520 |
| ctgcagtctt cagagggcca ccgcagctcc agtgccacgg caggaggctg ttcctgaata | 2580 |
| gcccctgtgg taagggccag gagagtcctt ccatcctcca aggccctgct aaaggacaca | 2640 |
| gcagccagga agtcccctgg gcccctagct gaaggacagc ctgctccctc cgtctctacc | 2700 |
| aggaatggcc ttgtcctatg gaaggcactg ccccatccca aactaatcta ggaatcactg | 2760 |
| tctaaccact cactgtcatg aatgtgtact taaaggatga ggttgagtca taccaaatag | 2820 |
| tgatttcgat agttcaaaat ggtgaaatta gcaattctac atgattcagt ctaatcaatg | 2880 |
| gataccgact gtttcccaca caagtctcct gttctcttaa gcttactcac tgacagcctt | 2940 |
| tcactctcca caaatacatt aaagatatgg ccatcaccaa gccccctagg atgacaccag | 3000 |
| acctgagagt ctgaagacct ggatccaagt tctgactttt ccccctgaca gctgtgtgac | 3060 |
| cttcgtgaag tcgccaaacc tctctgagcc ccagtcattg ctagtaagac ctgcctttga | 3120 |
| gttggtatga tgttcaagtt agataacaaa atgtttatac ccattagaac agagaataaa | 3180 |
| tagaactaca tttcttgca | 3199 |
| <210> 3 | |
| <211> 3513 | |
| <212> DNA | |
| <213> Homo sapiens | |
| <400> 3 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaagggcg | 240 |
| gcagtaagtc ttcagcatca ggcattttgg ggtgactcag taaatggtag atcttgctac | 300 |
| cagtggaaca gccactaagg attctgcagt gagagcagag ggccagctaa gtggtactct | 360 |
| cccagagact gtctgactca cgccaccccc tccaccttgg acacaggacg ctgtggtttc | 420 |
| tgagccaggt acaatgactc ctttcgcagc ctcccccgtt gcccctctgg atccactgct | 480 |
| taaatacgga cgaggacagg gccctgtctc ctcagcttca ggcaccacca ctgacctggg | 540 |
| acagtgaatc gacaatgccg tcttctgtct cgtggggcat cctcctgctg gcaggcctgt | 600 |
| gctgcctggt ccctgtctcc ctggctgagg atccccaggg agatgctgcc cagaagacag | 660 |
| atacatccca ccatgatcag gatcacccaa ccttcaacaa gatcaccccc aacctggctg | 720 |
| agttcgcctt cagcctatac cgccagctgg cacaccagtc caacagcacc aatatcttct | 780 |
| tctccccagt gagcatcgct acagcctttg caatgctctc cctggggacc aaggctgaca | 840 |
| ctcacgatga aatcctggag ggcctgaatt tcaacctcac ggagattccg gaggctcaga | 900 |
| tccatgaagg cttccaggaa ctcctccgta ccctcaacca gccagacagc cagctccagc | 960 |
| tgaccaccgg caatggcctg ttcctcagcg agggcctgaa gctagtggat aagtttttgg | 1020 |
| aggatgttaa aaagttgtac cactcagaag ccttcactgt caacttcggg gacaccgaag | 1080 |
| aggccaagaa acagatcaac gattacgtgg agaagggtac tcaagggaaa attgtggatt | 1140 |
| tggtcaagga gcttgacaga gacacagttt ttgctctggt gaattacatc ttctttaaag | 1200 |
| gcaaatggga gagacccttt gaagtcaagg acaccgagga agaggacttc cacgtggacc | 1260 |
| aggtgaccac cgtgaaggtg cctatgatga agcgtttagg catgtttaac atccagcact | 1320 |
| gtaagaagct gtccagctgg gtgctgctga tgaaatacct gggcaatgcc accgccatct | 1380 |
| tcttcctgcc tgatgagggg aaactacagc acctggaaaa tgaactcacc cacgatatca | 1440 |
| tcaccaagtt cctggaaaat gaagacagaa ggtctgccag cttacattta cccaaactgt | 1500 |
| ccattactgg aacctatgat ctgaagagcg tcctgggtca actgggcatc actaaggtct | 1560 |
| tcagcaatgg ggctgacctc tccggggtca cagaggaggc acccctgaag ctctccaagg | 1620 |
| ccgtgcataa ggctgtgctg accatcgacg agaaagggac tgaagctgct ggggccatgt | 1680 |
| ttttagaggc catacccatg tctatccccc ccgaggtcaa gttcaacaaa ccctttgtct | 1740 |
| tcttaatgat tgaacaaaat accaagtctc ccctcttcat gggaaaagtg gtgaatccca | 1800 |
| cccaaaaata actgcctctc gctcctcaac ccctcccctc catccctggc cccctccctg | 1860 |
| gatgacatta aagaagggtt gagctggtcc ctgcctgcat gtgactgtaa atccctccca | 1920 |
| tgttttctct gagtctccct ttgcctgctg aggctgtatg tgggctccag gtaacagtgc | 1980 |
| tgtcttcggg ccccctgaac tgtgttcatg gagcatctgg ctgggtaggc acatgctggg | 2040 |
| cttgaatcca ggggggactg aatcctcagc ttacggacct gggcccatct gtttctggag | 2100 |
| ggctccagtc ttccttgtcc tgtcttggag tccccaagaa ggaatcacag gggaggaacc | 2160 |
| agataccagc catgacccca ggctccacca agcatcttca tgtccccctg ctcatccccc | 2220 |
| actccccccc acccagagtt gctcatcctg ccagggctgg ctgtgcccac cccaaggctg | 2280 |
| ccctcctggg ggccccagaa ctgcctgatc gtgccgtggc ccagttttgt ggcatctgca | 2340 |
| gcaacacaag agagaggaca atgtcctcct cttgacccgc tgtcacctaa ccagactcgg | 2400 |
| gccctgcacc tctcaggcac ttctggaaaa tgactgaggc agattcttcc tgaagcccat | 2460 |
| tctccatggg gcaacaagga cacctattct gtccttgtcc ttccatcgct gccccagaaa | 2520 |
| gcctcacata tctccgttta gaatcaggtc ccttctcccc agatgaagag gagggtctct | 2580 |
| gctttgtttt ctctatctcc tcctcagact tgaccaggcc cagcaggccc cagaagacca | 2640 |
| ttaccctata tcccttctcc tccctagtca catggccata ggcctgctga tggctcagga | 2700 |
| aggccattgc aaggactcct cagctatggg agaggaagca catcacccat tgacccccgc | 2760 |
| aacccctccc tttcctcctc tgagtcccga ctggggccac atgcagcctg acttctttgt | 2820 |
| gcctgttgct gtccctgcag tcttcagagg gccaccgcag ctccagtgcc acggcaggag | 2880 |
| gctgttcctg aatagcccct gtggtaaggg ccaggagagt ccttccatcc tccaaggccc | 2940 |
| tgctaaagga cacagcagcc aggaagtccc ctgggcccct agctgaagga cagcctgctc | 3000 |
| cctccgtctc taccaggaat ggccttgtcc tatggaaggc actgccccat cccaaactaa | 3060 |
| tctaggaatc actgtctaac cactcactgt catgaatgtg tacttaaagg atgaggttga | 3120 |
| gtcataccaa atagtgattt cgatagttca aaatggtgaa attagcaatt ctacatgatt | 3180 |
| cagtctaatc aatggatacc gactgtttcc cacacaagtc tcctgttctc ttaagcttac | 3240 |
| tcactgacag cctttcactc tccacaaata cattaaagat atggccatca ccaagccccc | 3300 |
| taggatgaca ccagacctga gagtctgaag acctggatcc aagttctgac ttttccccct | 3360 |
| gacagctgtg tgaccttcgt gaagtcgcca aacctctctg agccccagtc attgctagta | 3420 |
| agacctgcct ttgagttggt atgatgttca agttagataa caaaatgttt atacccatta | 3480 |
| gaacagagaa taaatagaac tacatttctt gca | 3513 |
| <210> 4 | |
| <211> 3236 | |
| <212> DNA | |
| <213> Homo sapiens | |
| <400> 4 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaaggtgg | 240 |
| gacattgctg ctgctgctca ctcagttcca caggacaatg ccgtcttctg tctcgtgggg | 300 |
| catcctcctg ctggcaggcc tgtgctgcct ggtccctgtc tccctggctg aggatcccca | 360 |
| gggagatgct gcccagaaga cagatacatc ccaccatgat caggatcacc caaccttcaa | 420 |
| caagatcacc cccaacctgg ctgagttcgc cttcagccta taccgccagc tggcacacca | 480 |
| gtccaacagc accaatatct tcttctcccc agtgagcatc gctacagcct ttgcaatgct | 540 |
| ctccctgggg accaaggctg acactcacga tgaaatcctg gagggcctga atttcaacct | 600 |
| cacggagatt ccggaggctc agatccatga aggcttccag gaactcctcc gtaccctcaa | 660 |
| ccagccagac agccagctcc agctgaccac cggcaatggc ctgttcctca gcgagggcct | 720 |
| gaagctagtg gataagtttt tggaggatgt taaaaagttg taccactcag aagccttcac | 780 |
| tgtcaacttc ggggacaccg aagaggccaa gaaacagatc aacgattacg tggagaaggg | 840 |
| tactcaaggg aaaattgtgg atttggtcaa ggagcttgac agagacacag tttttgctct | 900 |
| ggtgaattac atcttcttta aaggcaaatg ggagagaccc tttgaagtca aggacaccga | 960 |
| ggaagaggac ttccacgtgg accaggtgac caccgtgaag gtgcctatga tgaagcgttt | 1020 |
| aggcatgttt aacatccagc actgtaagaa gctgtccagc tgggtgctgc tgatgaaata | 1080 |
| cctgggcaat gccaccgcca tcttcttcct gcctgatgag gggaaactac agcacctgga | 1140 |
| aaatgaactc acccacgata tcatcaccaa gttcctggaa aatgaagaca gaaggtctgc | 1200 |
| cagcttacat ttacccaaac tgtccattac tggaacctat gatctgaaga gcgtcctggg | 1260 |
| tcaactgggc atcactaagg tcttcagcaa tggggctgac ctctccgggg tcacagagga | 1320 |
| ggcacccctg aagctctcca aggccgtgca taaggctgtg ctgaccatcg acgagaaagg | 1380 |
| gactgaagct gctggggcca tgtttttaga ggccataccc atgtctatcc cccccgaggt | 1440 |
| caagttcaac aaaccctttg tcttcttaat gattgaacaa aataccaagt ctcccctctt | 1500 |
| catgggaaaa gtggtgaatc ccacccaaaa ataactgcct ctcgctcctc aacccctccc | 1560 |
| ctccatccct ggccccctcc ctggatgaca ttaaagaagg gttgagctgg tccctgcctg | 1620 |
| catgtgactg taaatccctc ccatgttttc tctgagtctc cctttgcctg ctgaggctgt | 1680 |
| atgtgggctc caggtaacag tgctgtcttc gggccccctg aactgtgttc atggagcatc | 1740 |
| tggctgggta ggcacatgct gggcttgaat ccagggggga ctgaatcctc agcttacgga | 1800 |
| cctgggccca tctgtttctg gagggctcca gtcttccttg tcctgtcttg gagtccccaa | 1860 |
| gaaggaatca caggggagga accagatacc agccatgacc ccaggctcca ccaagcatct | 1920 |
| tcatgtcccc ctgctcatcc cccactcccc cccacccaga gttgctcatc ctgccagggc | 1980 |
| tggctgtgcc caccccaagg ctgccctcct gggggcccca gaactgcctg atcgtgccgt | 2040 |
| ggcccagttt tgtggcatct gcagcaacac aagagagagg acaatgtcct cctcttgacc | 2100 |
| cgctgtcacc taaccagact cgggccctgc acctctcagg cacttctgga aaatgactga | 2160 |
| ggcagattct tcctgaagcc cattctccat ggggcaacaa ggacacctat tctgtccttg | 2220 |
| tccttccatc gctgccccag aaagcctcac atatctccgt ttagaatcag gtcccttctc | 2280 |
| cccagatgaa gaggagggtc tctgctttgt tttctctatc tcctcctcag acttgaccag | 2340 |
| gcccagcagg ccccagaaga ccattaccct atatcccttc tcctccctag tcacatggcc | 2400 |
| ataggcctgc tgatggctca ggaaggccat tgcaaggact cctcagctat gggagaggaa | 2460 |
| gcacatcacc cattgacccc cgcaacccct ccctttcctc ctctgagtcc cgactggggc | 2520 |
| cacatgcagc ctgacttctt tgtgcctgtt gctgtccctg cagtcttcag agggccaccg | 2580 |
| cagctccagt gccacggcag gaggctgttc ctgaatagcc cctgtggtaa gggccaggag | 2640 |
| agtccttcca tcctccaagg ccctgctaaa ggacacagca gccaggaagt cccctgggcc | 2700 |
| cctagctgaa ggacagcctg ctccctccgt ctctaccagg aatggccttg tcctatggaa | 2760 |
| ggcactgccc catcccaaac taatctagga atcactgtct aaccactcac tgtcatgaat | 2820 |
| gtgtacttaa aggatgaggt tgagtcatac caaatagtga tttcgatagt tcaaaatggt | 2880 |
| gaaattagca attctacatg attcagtcta atcaatggat accgactgtt tcccacacaa | 2940 |
| gtctcctgtt ctcttaagct tactcactga cagcctttca ctctccacaa atacattaaa | 3000 |
| gatatggcca tcaccaagcc ccctaggatg acaccagacc tgagagtctg aagacctgga | 3060 |
| tccaagttct gacttttccc cctgacagct gtgtgacctt cgtgaagtcg ccaaacctct | 3120 |
| ctgagcccca gtcattgcta gtaagacctg cctttgagtt ggtatgatgt tcaagttaga | 3180 |
| taacaaaatg tttataccca ttagaacaga gaataaatag aactacattt cttgca | 3236 |
| <210> 5 | |
| <211> 3532 | |
| <212> DNA | |
| <213> Homo sapiens | |
| <400> 5 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaaggtgg | 240 |
| gacattgctg ctgctgctca ctcagttcca cagggcggca gtaagtcttc agcatcaggc | 300 |
| attttggggt gactcagtaa atggtagatc ttgctaccag tggaacagcc actaaggatt | 360 |
| ctgcagtgag agcagagggc cagctaagtg gtactctccc agagactgtc tgactcacgc | 420 |
| caccccctcc accttggaca caggacgctg tggtttctga gccagcagcc tcccccgttg | 480 |
| cccctctgga tccactgctt aaatacggac gaggacaggg ccctgtctcc tcagcttcag | 540 |
| gcaccaccac tgacctggga cagtgaatcg acaatgccgt cttctgtctc gtggggcatc | 600 |
| ctcctgctgg caggcctgtg ctgcctggtc cctgtctccc tggctgagga tccccaggga | 660 |
| gatgctgccc agaagacaga tacatcccac catgatcagg atcacccaac cttcaacaag | 720 |
| atcaccccca acctggctga gttcgccttc agcctatacc gccagctggc acaccagtcc | 780 |
| aacagcacca atatcttctt ctccccagtg agcatcgcta cagcctttgc aatgctctcc | 840 |
| ctggggacca aggctgacac tcacgatgaa atcctggagg gcctgaattt caacctcacg | 900 |
| gagattccgg aggctcagat ccatgaaggc ttccaggaac tcctccgtac cctcaaccag | 960 |
| ccagacagcc agctccagct gaccaccggc aatggcctgt tcctcagcga gggcctgaag | 1020 |
| ctagtggata agtttttgga ggatgttaaa aagttgtacc actcagaagc cttcactgtc | 1080 |
| aacttcgggg acaccgaaga ggccaagaaa cagatcaacg attacgtgga gaagggtact | 1140 |
| caagggaaaa ttgtggattt ggtcaaggag cttgacagag acacagtttt tgctctggtg | 1200 |
| aattacatct tctttaaagg caaatgggag agaccctttg aagtcaagga caccgaggaa | 1260 |
| gaggacttcc acgtggacca ggtgaccacc gtgaaggtgc ctatgatgaa gcgtttaggc | 1320 |
| atgtttaaca tccagcactg taagaagctg tccagctggg tgctgctgat gaaatacctg | 1380 |
| ggcaatgcca ccgccatctt cttcctgcct gatgagggga aactacagca cctggaaaat | 1440 |
| gaactcaccc acgatatcat caccaagttc ctggaaaatg aagacagaag gtctgccagc | 1500 |
| ttacatttac ccaaactgtc cattactgga acctatgatc tgaagagcgt cctgggtcaa | 1560 |
| ctgggcatca ctaaggtctt cagcaatggg gctgacctct ccggggtcac agaggaggca | 1620 |
| cccctgaagc tctccaaggc cgtgcataag gctgtgctga ccatcgacga gaaagggact | 1680 |
| gaagctgctg gggccatgtt tttagaggcc atacccatgt ctatcccccc cgaggtcaag | 1740 |
| ttcaacaaac cctttgtctt cttaatgatt gaacaaaata ccaagtctcc cctcttcatg | 1800 |
| ggaaaagtgg tgaatcccac ccaaaaataa ctgcctctcg ctcctcaacc cctcccctcc | 1860 |
| atccctggcc ccctccctgg atgacattaa agaagggttg agctggtccc tgcctgcatg | 1920 |
| tgactgtaaa tccctcccat gttttctctg agtctccctt tgcctgctga ggctgtatgt | 1980 |
| gggctccagg taacagtgct gtcttcgggc cccctgaact gtgttcatgg agcatctggc | 2040 |
| tgggtaggca catgctgggc ttgaatccag gggggactga atcctcagct tacggacctg | 2100 |
| ggcccatctg tttctggagg gctccagtct tccttgtcct gtcttggagt ccccaagaag | 2160 |
| gaatcacagg ggaggaacca gataccagcc atgaccccag gctccaccaa gcatcttcat | 2220 |
| gtccccctgc tcatccccca ctccccccca cccagagttg ctcatcctgc cagggctggc | 2280 |
| tgtgcccacc ccaaggctgc cctcctgggg gccccagaac tgcctgatcg tgccgtggcc | 2340 |
| cagttttgtg gcatctgcag caacacaaga gagaggacaa tgtcctcctc ttgacccgct | 2400 |
| gtcacctaac cagactcggg ccctgcacct ctcaggcact tctggaaaat gactgaggca | 2460 |
| gattcttcct gaagcccatt ctccatgggg caacaaggac acctattctg tccttgtcct | 2520 |
| tccatcgctg ccccagaaag cctcacatat ctccgtttag aatcaggtcc cttctcccca | 2580 |
| gatgaagagg agggtctctg ctttgttttc tctatctcct cctcagactt gaccaggccc | 2640 |
| agcaggcccc agaagaccat taccctatat cccttctcct ccctagtcac atggccatag | 2700 |
| gcctgctgat ggctcaggaa ggccattgca aggactcctc agctatggga gaggaagcac | 2760 |
| atcacccatt gacccccgca acccctccct ttcctcctct gagtcccgac tggggccaca | 2820 |
| tgcagcctga cttctttgtg cctgttgctg tccctgcagt cttcagaggg ccaccgcagc | 2880 |
| tccagtgcca cggcaggagg ctgttcctga atagcccctg tggtaagggc caggagagtc | 2940 |
| cttccatcct ccaaggccct gctaaaggac acagcagcca ggaagtcccc tgggccccta | 3000 |
| gctgaaggac agcctgctcc ctccgtctct accaggaatg gccttgtcct atggaaggca | 3060 |
| ctgccccatc ccaaactaat ctaggaatca ctgtctaacc actcactgtc atgaatgtgt | 3120 |
| acttaaagga tgaggttgag tcataccaaa tagtgatttc gatagttcaa aatggtgaaa | 3180 |
| ttagcaattc tacatgattc agtctaatca atggataccg actgtttccc acacaagtct | 3240 |
| cctgttctct taagcttact cactgacagc ctttcactct ccacaaatac attaaagata | 3300 |
| tggccatcac caagccccct aggatgacac cagacctgag agtctgaaga cctggatcca | 3360 |
| agttctgact tttccccctg acagctgtgt gaccttcgtg aagtcgccaa acctctctga | 3420 |
| gccccagtca ttgctagtaa gacctgcctt tgagttggta tgatgttcaa gttagataac | 3480 |
| aaaatgttta tacccattag aacagagaat aaatagaact acatttcttg ca | 3532 |
| <210> 6 | |
| <211> 3340 | |
| <212> DNA | |
| <213> Homo sapiens | |
| <400> 6 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaaggtgg | 240 |
| gacattgctg ctgctgctca ctcagttcca cagcagcctc ccccgttgcc cctctggatc | 300 |
| cactgcttaa atacggacga ggacagggcc ctgtctcctc agcttcaggc accaccactg | 360 |
| acctgggaca gtgaatcgac aatgccgtct tctgtctcgt ggggcatcct cctgctggca | 420 |
| ggcctgtgct gcctggtccc tgtctccctg gctgaggatc cccagggaga tgctgcccag | 480 |
| aagacagata catcccacca tgatcaggat cacccaacct tcaacaagat cacccccaac | 540 |
| ctggctgagt tcgccttcag cctataccgc cagctggcac accagtccaa cagcaccaat | 600 |
| atcttcttct ccccagtgag catcgctaca gcctttgcaa tgctctccct ggggaccaag | 660 |
| gctgacactc acgatgaaat cctggagggc ctgaatttca acctcacgga gattccggag | 720 |
| gctcagatcc atgaaggctt ccaggaactc ctccgtaccc tcaaccagcc agacagccag | 780 |
| ctccagctga ccaccggcaa tggcctgttc ctcagcgagg gcctgaagct agtggataag | 840 |
| tttttggagg atgttaaaaa gttgtaccac tcagaagcct tcactgtcaa cttcggggac | 900 |
| accgaagagg ccaagaaaca gatcaacgat tacgtggaga agggtactca agggaaaatt | 960 |
| gtggatttgg tcaaggagct tgacagagac acagtttttg ctctggtgaa ttacatcttc | 1020 |
| tttaaaggca aatgggagag accctttgaa gtcaaggaca ccgaggaaga ggacttccac | 1080 |
| gtggaccagg tgaccaccgt gaaggtgcct atgatgaagc gtttaggcat gtttaacatc | 1140 |
| cagcactgta agaagctgtc cagctgggtg ctgctgatga aatacctggg caatgccacc | 1200 |
| gccatcttct tcctgcctga tgaggggaaa ctacagcacc tggaaaatga actcacccac | 1260 |
| gatatcatca ccaagttcct ggaaaatgaa gacagaaggt ctgccagctt acatttaccc | 1320 |
| aaactgtcca ttactggaac ctatgatctg aagagcgtcc tgggtcaact gggcatcact | 1380 |
| aaggtcttca gcaatggggc tgacctctcc ggggtcacag aggaggcacc cctgaagctc | 1440 |
| tccaaggccg tgcataaggc tgtgctgacc atcgacgaga aagggactga agctgctggg | 1500 |
| gccatgtttt tagaggccat acccatgtct atcccccccg aggtcaagtt caacaaaccc | 1560 |
| tttgtcttct taatgattga acaaaatacc aagtctcccc tcttcatggg aaaagtggtg | 1620 |
| aatcccaccc aaaaataact gcctctcgct cctcaacccc tcccctccat ccctggcccc | 1680 |
| ctccctggat gacattaaag aagggttgag ctggtccctg cctgcatgtg actgtaaatc | 1740 |
| cctcccatgt tttctctgag tctccctttg cctgctgagg ctgtatgtgg gctccaggta | 1800 |
| acagtgctgt cttcgggccc cctgaactgt gttcatggag catctggctg ggtaggcaca | 1860 |
| tgctgggctt gaatccaggg gggactgaat cctcagctta cggacctggg cccatctgtt | 1920 |
| tctggagggc tccagtcttc cttgtcctgt cttggagtcc ccaagaagga atcacagggg | 1980 |
| aggaaccaga taccagccat gaccccaggc tccaccaagc atcttcatgt ccccctgctc | 2040 |
| atcccccact cccccccacc cagagttgct catcctgcca gggctggctg tgcccacccc | 2100 |
| aaggctgccc tcctgggggc cccagaactg cctgatcgtg ccgtggccca gttttgtggc | 2160 |
| atctgcagca acacaagaga gaggacaatg tcctcctctt gacccgctgt cacctaacca | 2220 |
| gactcgggcc ctgcacctct caggcacttc tggaaaatga ctgaggcaga ttcttcctga | 2280 |
| agcccattct ccatggggca acaaggacac ctattctgtc cttgtccttc catcgctgcc | 2340 |
| ccagaaagcc tcacatatct ccgtttagaa tcaggtccct tctccccaga tgaagaggag | 2400 |
| ggtctctgct ttgttttctc tatctcctcc tcagacttga ccaggcccag caggccccag | 2460 |
| aagaccatta ccctatatcc cttctcctcc ctagtcacat ggccataggc ctgctgatgg | 2520 |
| ctcaggaagg ccattgcaag gactcctcag ctatgggaga ggaagcacat cacccattga | 2580 |
| cccccgcaac ccctcccttt cctcctctga gtcccgactg gggccacatg cagcctgact | 2640 |
| tctttgtgcc tgttgctgtc cctgcagtct tcagagggcc accgcagctc cagtgccacg | 2700 |
| gcaggaggct gttcctgaat agcccctgtg gtaagggcca ggagagtcct tccatcctcc | 2760 |
| aaggccctgc taaaggacac agcagccagg aagtcccctg ggcccctagc tgaaggacag | 2820 |
| cctgctccct ccgtctctac caggaatggc cttgtcctat ggaaggcact gccccatccc | 2880 |
| aaactaatct aggaatcact gtctaaccac tcactgtcat gaatgtgtac ttaaaggatg | 2940 |
| aggttgagtc ataccaaata gtgatttcga tagttcaaaa tggtgaaatt agcaattcta | 3000 |
| catgattcag tctaatcaat ggataccgac tgtttcccac acaagtctcc tgttctctta | 3060 |
| agcttactca ctgacagcct ttcactctcc acaaatacat taaagatatg gccatcacca | 3120 |
| agccccctag gatgacacca gacctgagag tctgaagacc tggatccaag ttctgacttt | 3180 |
| tccccctgac agctgtgtga ccttcgtgaa gtcgccaaac ctctctgagc cccagtcatt | 3240 |
| gctagtaaga cctgcctttg agttggtatg atgttcaagt tagataacaa aatgtttata | 3300 |
| cccattagaa cagagaataa atagaactac atttcttgca | 3340 |
| <210> 7 | |
| <211> 3495 | |
| <212> DNA | |
| <213> Homo sapiens | |
| <400> 7 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaagggcg | 240 |
| gcagtaagtc ttcagcatca ggcattttgg ggtgactcag taaatggtag atcttgctac | 300 |
| cagtggaaca gccactaagg attctgcagt gagagcagag ggccagctaa gtggtactct | 360 |
| cccagagact gtctgactca cgccaccccc tccaccttgg acacaggacg ctgtggtttc | 420 |
| tgagccagca gcctcccccg ttgcccctct ggatccactg cttaaatacg gacgaggaca | 480 |
| gggccctgtc tcctcagctt caggcaccac cactgacctg ggacagtgaa tcgacaatgc | 540 |
| cgtcttctgt ctcgtggggc atcctcctgc tggcaggcct gtgctgcctg gtccctgtct | 600 |
| ccctggctga ggatccccag ggagatgctg cccagaagac agatacatcc caccatgatc | 660 |
| aggatcaccc aaccttcaac aagatcaccc ccaacctggc tgagttcgcc ttcagcctat | 720 |
| accgccagct ggcacaccag tccaacagca ccaatatctt cttctcccca gtgagcatcg | 780 |
| ctacagcctt tgcaatgctc tccctgggga ccaaggctga cactcacgat gaaatcctgg | 840 |
| agggcctgaa tttcaacctc acggagattc cggaggctca gatccatgaa ggcttccagg | 900 |
| aactcctccg taccctcaac cagccagaca gccagctcca gctgaccacc ggcaatggcc | 960 |
| tgttcctcag cgagggcctg aagctagtgg ataagttttt ggaggatgtt aaaaagttgt | 1020 |
| accactcaga agccttcact gtcaacttcg gggacaccga agaggccaag aaacagatca | 1080 |
| acgattacgt ggagaagggt actcaaggga aaattgtgga tttggtcaag gagcttgaca | 1140 |
| gagacacagt ttttgctctg gtgaattaca tcttctttaa aggcaaatgg gagagaccct | 1200 |
| ttgaagtcaa ggacaccgag gaagaggact tccacgtgga ccaggtgacc accgtgaagg | 1260 |
| tgcctatgat gaagcgttta ggcatgttta acatccagca ctgtaagaag ctgtccagct | 1320 |
| gggtgctgct gatgaaatac ctgggcaatg ccaccgccat cttcttcctg cctgatgagg | 1380 |
| ggaaactaca gcacctggaa aatgaactca cccacgatat catcaccaag ttcctggaaa | 1440 |
| atgaagacag aaggtctgcc agcttacatt tacccaaact gtccattact ggaacctatg | 1500 |
| atctgaagag cgtcctgggt caactgggca tcactaaggt cttcagcaat ggggctgacc | 1560 |
| tctccggggt cacagaggag gcacccctga agctctccaa ggccgtgcat aaggctgtgc | 1620 |
| tgaccatcga cgagaaaggg actgaagctg ctggggccat gtttttagag gccataccca | 1680 |
| tgtctatccc ccccgaggtc aagttcaaca aaccctttgt cttcttaatg attgaacaaa | 1740 |
| ataccaagtc tcccctcttc atgggaaaag tggtgaatcc cacccaaaaa taactgcctc | 1800 |
| tcgctcctca acccctcccc tccatccctg gccccctccc tggatgacat taaagaaggg | 1860 |
| ttgagctggt ccctgcctgc atgtgactgt aaatccctcc catgttttct ctgagtctcc | 1920 |
| ctttgcctgc tgaggctgta tgtgggctcc aggtaacagt gctgtcttcg ggccccctga | 1980 |
| actgtgttca tggagcatct ggctgggtag gcacatgctg ggcttgaatc caggggggac | 2040 |
| tgaatcctca gcttacggac ctgggcccat ctgtttctgg agggctccag tcttccttgt | 2100 |
| cctgtcttgg agtccccaag aaggaatcac aggggaggaa ccagatacca gccatgaccc | 2160 |
| caggctccac caagcatctt catgtccccc tgctcatccc ccactccccc ccacccagag | 2220 |
| ttgctcatcc tgccagggct ggctgtgccc accccaaggc tgccctcctg ggggccccag | 2280 |
| aactgcctga tcgtgccgtg gcccagtttt gtggcatctg cagcaacaca agagagagga | 2340 |
| caatgtcctc ctcttgaccc gctgtcacct aaccagactc gggccctgca cctctcaggc | 2400 |
| acttctggaa aatgactgag gcagattctt cctgaagccc attctccatg gggcaacaag | 2460 |
| gacacctatt ctgtccttgt ccttccatcg ctgccccaga aagcctcaca tatctccgtt | 2520 |
| tagaatcagg tcccttctcc ccagatgaag aggagggtct ctgctttgtt ttctctatct | 2580 |
| cctcctcaga cttgaccagg cccagcaggc cccagaagac cattacccta tatcccttct | 2640 |
| cctccctagt cacatggcca taggcctgct gatggctcag gaaggccatt gcaaggactc | 2700 |
| ctcagctatg ggagaggaag cacatcaccc attgaccccc gcaacccctc cctttcctcc | 2760 |
| tctgagtccc gactggggcc acatgcagcc tgacttcttt gtgcctgttg ctgtccctgc | 2820 |
| agtcttcaga gggccaccgc agctccagtg ccacggcagg aggctgttcc tgaatagccc | 2880 |
| ctgtggtaag ggccaggaga gtccttccat cctccaaggc cctgctaaag gacacagcag | 2940 |
| ccaggaagtc ccctgggccc ctagctgaag gacagcctgc tccctccgtc tctaccagga | 3000 |
| atggccttgt cctatggaag gcactgcccc atcccaaact aatctaggaa tcactgtcta | 3060 |
| accactcact gtcatgaatg tgtacttaaa ggatgaggtt gagtcatacc aaatagtgat | 3120 |
| ttcgatagtt caaaatggtg aaattagcaa ttctacatga ttcagtctaa tcaatggata | 3180 |
| ccgactgttt cccacacaag tctcctgttc tcttaagctt actcactgac agcctttcac | 3240 |
| tctccacaaa tacattaaag atatggccat caccaagccc cctaggatga caccagacct | 3300 |
| gagagtctga agacctggat ccaagttctg acttttcccc ctgacagctg tgtgaccttc | 3360 |
| gtgaagtcgc caaacctctc tgagccccag tcattgctag taagacctgc ctttgagttg | 3420 |
| gtatgatgtt caagttagat aacaaaatgt ttatacccat tagaacagag aataaataga | 3480 |
| actacatttc ttgca | 3495 |
| <210> 8 | |
| <211> 3492 | |
| <212> DNA | |
| <213> Homo sapiens | |
| <400> 8 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaagggcg | 240 |
| gcagtaagtc ttcagcatca ggcattttgg ggtgactcag taaatggtag atcttgctac | 300 |
| cagtggaaca gccactaagg attctgcagt gagagcagag ggccagctaa gtggtactct | 360 |
| cccagagact gtctgactca cgccaccccc tccaccttgg acacaggacg ctgtggtttc | 420 |
| tgagccagcc tcccccgttg cccctctgga tccactgctt aaatacggac gaggacaggg | 480 |
| ccctgtctcc tcagcttcag gcaccaccac tgacctggga cagtgaatcg acaatgccgt | 540 |
| cttctgtctc gtggggcatc ctcctgctgg caggcctgtg ctgcctggtc cctgtctccc | 600 |
| tggctgagga tccccaggga gatgctgccc agaagacaga tacatcccac catgatcagg | 660 |
| atcacccaac cttcaacaag atcaccccca acctggctga gttcgccttc agcctatacc | 720 |
| gccagctggc acaccagtcc aacagcacca atatcttctt ctccccagtg agcatcgcta | 780 |
| cagcctttgc aatgctctcc ctggggacca aggctgacac tcacgatgaa atcctggagg | 840 |
| gcctgaattt caacctcacg gagattccgg aggctcagat ccatgaaggc ttccaggaac | 900 |
| tcctccgtac cctcaaccag ccagacagcc agctccagct gaccaccggc aatggcctgt | 960 |
| tcctcagcga gggcctgaag ctagtggata agtttttgga ggatgttaaa aagttgtacc | 1020 |
| actcagaagc cttcactgtc aacttcgggg acaccgaaga ggccaagaaa cagatcaacg | 1080 |
| attacgtgga gaagggtact caagggaaaa ttgtggattt ggtcaaggag cttgacagag | 1140 |
| acacagtttt tgctctggtg aattacatct tctttaaagg caaatgggag agaccctttg | 1200 |
| aagtcaagga caccgaggaa gaggacttcc acgtggacca ggtgaccacc gtgaaggtgc | 1260 |
| ctatgatgaa gcgtttaggc atgtttaaca tccagcactg taagaagctg tccagctggg | 1320 |
| tgctgctgat gaaatacctg ggcaatgcca ccgccatctt cttcctgcct gatgagggga | 1380 |
| aactacagca cctggaaaat gaactcaccc acgatatcat caccaagttc ctggaaaatg | 1440 |
| aagacagaag gtctgccagc ttacatttac ccaaactgtc cattactgga acctatgatc | 1500 |
| tgaagagcgt cctgggtcaa ctgggcatca ctaaggtctt cagcaatggg gctgacctct | 1560 |
| ccggggtcac agaggaggca cccctgaagc tctccaaggc cgtgcataag gctgtgctga | 1620 |
| ccatcgacga gaaagggact gaagctgctg gggccatgtt tttagaggcc atacccatgt | 1680 |
| ctatcccccc cgaggtcaag ttcaacaaac cctttgtctt cttaatgatt gaacaaaata | 1740 |
| ccaagtctcc cctcttcatg ggaaaagtgg tgaatcccac ccaaaaataa ctgcctctcg | 1800 |
| ctcctcaacc cctcccctcc atccctggcc ccctccctgg atgacattaa agaagggttg | 1860 |
| agctggtccc tgcctgcatg tgactgtaaa tccctcccat gttttctctg agtctccctt | 1920 |
| tgcctgctga ggctgtatgt gggctccagg taacagtgct gtcttcgggc cccctgaact | 1980 |
| gtgttcatgg agcatctggc tgggtaggca catgctgggc ttgaatccag gggggactga | 2040 |
| atcctcagct tacggacctg ggcccatctg tttctggagg gctccagtct tccttgtcct | 2100 |
| gtcttggagt ccccaagaag gaatcacagg ggaggaacca gataccagcc atgaccccag | 2160 |
| gctccaccaa gcatcttcat gtccccctgc tcatccccca ctccccccca cccagagttg | 2220 |
| ctcatcctgc cagggctggc tgtgcccacc ccaaggctgc cctcctgggg gccccagaac | 2280 |
| tgcctgatcg tgccgtggcc cagttttgtg gcatctgcag caacacaaga gagaggacaa | 2340 |
| tgtcctcctc ttgacccgct gtcacctaac cagactcggg ccctgcacct ctcaggcact | 2400 |
| tctggaaaat gactgaggca gattcttcct gaagcccatt ctccatgggg caacaaggac | 2460 |
| acctattctg tccttgtcct tccatcgctg ccccagaaag cctcacatat ctccgtttag | 2520 |
| aatcaggtcc cttctcccca gatgaagagg agggtctctg ctttgttttc tctatctcct | 2580 |
| cctcagactt gaccaggccc agcaggcccc agaagaccat taccctatat cccttctcct | 2640 |
| ccctagtcac atggccatag gcctgctgat ggctcaggaa ggccattgca aggactcctc | 2700 |
| agctatggga gaggaagcac atcacccatt gacccccgca acccctccct ttcctcctct | 2760 |
| gagtcccgac tggggccaca tgcagcctga cttctttgtg cctgttgctg tccctgcagt | 2820 |
| cttcagaggg ccaccgcagc tccagtgcca cggcaggagg ctgttcctga atagcccctg | 2880 |
| tggtaagggc caggagagtc cttccatcct ccaaggccct gctaaaggac acagcagcca | 2940 |
| ggaagtcccc tgggccccta gctgaaggac agcctgctcc ctccgtctct accaggaatg | 3000 |
| gccttgtcct atggaaggca ctgccccatc ccaaactaat ctaggaatca ctgtctaacc | 3060 |
| actcactgtc atgaatgtgt acttaaagga tgaggttgag tcataccaaa tagtgatttc | 3120 |
| gatagttcaa aatggtgaaa ttagcaattc tacatgattc agtctaatca atggataccg | 3180 |
| actgtttccc acacaagtct cctgttctct taagcttact cactgacagc ctttcactct | 3240 |
| ccacaaatac attaaagata tggccatcac caagccccct aggatgacac cagacctgag | 3300 |
| agtctgaaga cctggatcca agttctgact tttccccctg acagctgtgt gaccttcgtg | 3360 |
| aagtcgccaa acctctctga gccccagtca ttgctagtaa gacctgcctt tgagttggta | 3420 |
| tgatgttcaa gttagataac aaaatgttta tacccattag aacagagaat aaatagaact | 3480 |
| acatttcttg ca | 3492 |
| <210> 9 | |
| <211> 3510 | |
| <212> DNA | |
| <213> Homo sapiens | |
| <400> 9 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaagggcg | 240 |
| gcagtaagtc ttcagcatca ggcattttgg ggtgactcag taaatggtag atcttgctac | 300 |
| cagtggaaca gccactaagg attctgcagt gagagcagag ggccagctaa gtggtactct | 360 |
| cccagagact gtctgactca cgccaccccc tccaccttgg acacaggacg ctgtggtttc | 420 |
| tgagccaggt acaatgactc ctttcgcctc ccccgttgcc cctctggatc cactgcttaa | 480 |
| atacggacga ggacagggcc ctgtctcctc agcttcaggc accaccactg acctgggaca | 540 |
| gtgaatcgac aatgccgtct tctgtctcgt ggggcatcct cctgctggca ggcctgtgct | 600 |
| gcctggtccc tgtctccctg gctgaggatc cccagggaga tgctgcccag aagacagata | 660 |
| catcccacca tgatcaggat cacccaacct tcaacaagat cacccccaac ctggctgagt | 720 |
| tcgccttcag cctataccgc cagctggcac accagtccaa cagcaccaat atcttcttct | 780 |
| ccccagtgag catcgctaca gcctttgcaa tgctctccct ggggaccaag gctgacactc | 840 |
| acgatgaaat cctggagggc ctgaatttca acctcacgga gattccggag gctcagatcc | 900 |
| atgaaggctt ccaggaactc ctccgtaccc tcaaccagcc agacagccag ctccagctga | 960 |
| ccaccggcaa tggcctgttc ctcagcgagg gcctgaagct agtggataag tttttggagg | 1020 |
| atgttaaaaa gttgtaccac tcagaagcct tcactgtcaa cttcggggac accgaagagg | 1080 |
| ccaagaaaca gatcaacgat tacgtggaga agggtactca agggaaaatt gtggatttgg | 1140 |
| tcaaggagct tgacagagac acagtttttg ctctggtgaa ttacatcttc tttaaaggca | 1200 |
| aatgggagag accctttgaa gtcaaggaca ccgaggaaga ggacttccac gtggaccagg | 1260 |
| tgaccaccgt gaaggtgcct atgatgaagc gtttaggcat gtttaacatc cagcactgta | 1320 |
| agaagctgtc cagctgggtg ctgctgatga aatacctggg caatgccacc gccatcttct | 1380 |
| tcctgcctga tgaggggaaa ctacagcacc tggaaaatga actcacccac gatatcatca | 1440 |
| ccaagttcct ggaaaatgaa gacagaaggt ctgccagctt acatttaccc aaactgtcca | 1500 |
| ttactggaac ctatgatctg aagagcgtcc tgggtcaact gggcatcact aaggtcttca | 1560 |
| gcaatggggc tgacctctcc ggggtcacag aggaggcacc cctgaagctc tccaaggccg | 1620 |
| tgcataaggc tgtgctgacc atcgacgaga aagggactga agctgctggg gccatgtttt | 1680 |
| tagaggccat acccatgtct atcccccccg aggtcaagtt caacaaaccc tttgtcttct | 1740 |
| taatgattga acaaaatacc aagtctcccc tcttcatggg aaaagtggtg aatcccaccc | 1800 |
| aaaaataact gcctctcgct cctcaacccc tcccctccat ccctggcccc ctccctggat | 1860 |
| gacattaaag aagggttgag ctggtccctg cctgcatgtg actgtaaatc cctcccatgt | 1920 |
| tttctctgag tctccctttg cctgctgagg ctgtatgtgg gctccaggta acagtgctgt | 1980 |
| cttcgggccc cctgaactgt gttcatggag catctggctg ggtaggcaca tgctgggctt | 2040 |
| gaatccaggg gggactgaat cctcagctta cggacctggg cccatctgtt tctggagggc | 2100 |
| tccagtcttc cttgtcctgt cttggagtcc ccaagaagga atcacagggg aggaaccaga | 2160 |
| taccagccat gaccccaggc tccaccaagc atcttcatgt ccccctgctc atcccccact | 2220 |
| cccccccacc cagagttgct catcctgcca gggctggctg tgcccacccc aaggctgccc | 2280 |
| tcctgggggc cccagaactg cctgatcgtg ccgtggccca gttttgtggc atctgcagca | 2340 |
| acacaagaga gaggacaatg tcctcctctt gacccgctgt cacctaacca gactcgggcc | 2400 |
| ctgcacctct caggcacttc tggaaaatga ctgaggcaga ttcttcctga agcccattct | 2460 |
| ccatggggca acaaggacac ctattctgtc cttgtccttc catcgctgcc ccagaaagcc | 2520 |
| tcacatatct ccgtttagaa tcaggtccct tctccccaga tgaagaggag ggtctctgct | 2580 |
| ttgttttctc tatctcctcc tcagacttga ccaggcccag caggccccag aagaccatta | 2640 |
| ccctatatcc cttctcctcc ctagtcacat ggccataggc ctgctgatgg ctcaggaagg | 2700 |
| ccattgcaag gactcctcag ctatgggaga ggaagcacat cacccattga cccccgcaac | 2760 |
| ccctcccttt cctcctctga gtcccgactg gggccacatg cagcctgact tctttgtgcc | 2820 |
| tgttgctgtc cctgcagtct tcagagggcc accgcagctc cagtgccacg gcaggaggct | 2880 |
| gttcctgaat agcccctgtg gtaagggcca ggagagtcct tccatcctcc aaggccctgc | 2940 |
| taaaggacac agcagccagg aagtcccctg ggcccctagc tgaaggacag cctgctccct | 3000 |
| ccgtctctac caggaatggc cttgtcctat ggaaggcact gccccatccc aaactaatct | 3060 |
| aggaatcact gtctaaccac tcactgtcat gaatgtgtac ttaaaggatg aggttgagtc | 3120 |
| ataccaaata gtgatttcga tagttcaaaa tggtgaaatt agcaattcta catgattcag | 3180 |
| tctaatcaat ggataccgac tgtttcccac acaagtctcc tgttctctta agcttactca | 3240 |
| ctgacagcct ttcactctcc acaaatacat taaagatatg gccatcacca agccccctag | 3300 |
| gatgacacca gacctgagag tctgaagacc tggatccaag ttctgacttt tccccctgac | 3360 |
| agctgtgtga ccttcgtgaa gtcgccaaac ctctctgagc cccagtcatt gctagtaaga | 3420 |
| cctgcctttg agttggtatg atgttcaagt tagataacaa aatgtttata cccattagaa | 3480 |
| cagagaataa atagaactac atttcttgca | 3510 |
| <210> 10 | |
| <211> 3303 | |
| <212> DNA | |
| <213> Homo sapiens | |
| <400> 10 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaagcagc | 240 |
| ctcccccgtt gcccctctgg atccactgct taaatacgga cgaggacagg gccctgtctc | 300 |
| ctcagcttca ggcaccacca ctgacctggg acagtgaatc gacaatgccg tcttctgtct | 360 |
| cgtggggcat cctcctgctg gcaggcctgt gctgcctggt ccctgtctcc ctggctgagg | 420 |
| atccccaggg agatgctgcc cagaagacag atacatccca ccatgatcag gatcacccaa | 480 |
| ccttcaacaa gatcaccccc aacctggctg agttcgcctt cagcctatac cgccagctgg | 540 |
| cacaccagtc caacagcacc aatatcttct tctccccagt gagcatcgct acagcctttg | 600 |
| caatgctctc cctggggacc aaggctgaca ctcacgatga aatcctggag ggcctgaatt | 660 |
| tcaacctcac ggagattccg gaggctcaga tccatgaagg cttccaggaa ctcctccgta | 720 |
| ccctcaacca gccagacagc cagctccagc tgaccaccgg caatggcctg ttcctcagcg | 780 |
| agggcctgaa gctagtggat aagtttttgg aggatgttaa aaagttgtac cactcagaag | 840 |
| ccttcactgt caacttcggg gacaccgaag aggccaagaa acagatcaac gattacgtgg | 900 |
| agaagggtac tcaagggaaa attgtggatt tggtcaagga gcttgacaga gacacagttt | 960 |
| ttgctctggt gaattacatc ttctttaaag gcaaatggga gagacccttt gaagtcaagg | 1020 |
| acaccgagga agaggacttc cacgtggacc aggtgaccac cgtgaaggtg cctatgatga | 1080 |
| agcgtttagg catgtttaac atccagcact gtaagaagct gtccagctgg gtgctgctga | 1140 |
| tgaaatacct gggcaatgcc accgccatct tcttcctgcc tgatgagggg aaactacagc | 1200 |
| acctggaaaa tgaactcacc cacgatatca tcaccaagtt cctggaaaat gaagacagaa | 1260 |
| ggtctgccag cttacattta cccaaactgt ccattactgg aacctatgat ctgaagagcg | 1320 |
| tcctgggtca actgggcatc actaaggtct tcagcaatgg ggctgacctc tccggggtca | 1380 |
| cagaggaggc acccctgaag ctctccaagg ccgtgcataa ggctgtgctg accatcgacg | 1440 |
| agaaagggac tgaagctgct ggggccatgt ttttagaggc catacccatg tctatccccc | 1500 |
| ccgaggtcaa gttcaacaaa ccctttgtct tcttaatgat tgaacaaaat accaagtctc | 1560 |
| ccctcttcat gggaaaagtg gtgaatccca cccaaaaata actgcctctc gctcctcaac | 1620 |
| ccctcccctc catccctggc cccctccctg gatgacatta aagaagggtt gagctggtcc | 1680 |
| ctgcctgcat gtgactgtaa atccctccca tgttttctct gagtctccct ttgcctgctg | 1740 |
| aggctgtatg tgggctccag gtaacagtgc tgtcttcggg ccccctgaac tgtgttcatg | 1800 |
| gagcatctgg ctgggtaggc acatgctggg cttgaatcca ggggggactg aatcctcagc | 1860 |
| ttacggacct gggcccatct gtttctggag ggctccagtc ttccttgtcc tgtcttggag | 1920 |
| tccccaagaa ggaatcacag gggaggaacc agataccagc catgacccca ggctccacca | 1980 |
| agcatcttca tgtccccctg ctcatccccc actccccccc acccagagtt gctcatcctg | 2040 |
| ccagggctgg ctgtgcccac cccaaggctg ccctcctggg ggccccagaa ctgcctgatc | 2100 |
| gtgccgtggc ccagttttgt ggcatctgca gcaacacaag agagaggaca atgtcctcct | 2160 |
| cttgacccgc tgtcacctaa ccagactcgg gccctgcacc tctcaggcac ttctggaaaa | 2220 |
| tgactgaggc agattcttcc tgaagcccat tctccatggg gcaacaagga cacctattct | 2280 |
| gtccttgtcc ttccatcgct gccccagaaa gcctcacata tctccgttta gaatcaggtc | 2340 |
| ccttctcccc agatgaagag gagggtctct gctttgtttt ctctatctcc tcctcagact | 2400 |
| tgaccaggcc cagcaggccc cagaagacca ttaccctata tcccttctcc tccctagtca | 2460 |
| catggccata ggcctgctga tggctcagga aggccattgc aaggactcct cagctatggg | 2520 |
| agaggaagca catcacccat tgacccccgc aacccctccc tttcctcctc tgagtcccga | 2580 |
| ctggggccac atgcagcctg acttctttgt gcctgttgct gtccctgcag tcttcagagg | 2640 |
| gccaccgcag ctccagtgcc acggcaggag gctgttcctg aatagcccct gtggtaaggg | 2700 |
| ccaggagagt ccttccatcc tccaaggccc tgctaaagga cacagcagcc aggaagtccc | 2760 |
| ctgggcccct agctgaagga cagcctgctc cctccgtctc taccaggaat ggccttgtcc | 2820 |
| tatggaaggc actgccccat cccaaactaa tctaggaatc actgtctaac cactcactgt | 2880 |
| catgaatgtg tacttaaagg atgaggttga gtcataccaa atagtgattt cgatagttca | 2940 |
| aaatggtgaa attagcaatt ctacatgatt cagtctaatc aatggatacc gactgtttcc | 3000 |
| cacacaagtc tcctgttctc ttaagcttac tcactgacag cctttcactc tccacaaata | 3060 |
| cattaaagat atggccatca ccaagccccc taggatgaca ccagacctga gagtctgaag | 3120 |
| acctggatcc aagttctgac ttttccccct gacagctgtg tgaccttcgt gaagtcgcca | 3180 |
| aacctctctg agccccagtc attgctagta agacctgcct ttgagttggt atgatgttca | 3240 |
| agttagataa caaaatgttt atacccatta gaacagagaa taaatagaac tacatttctt | 3300 |
| gca | 3303 |
| <210> 11 | |
| <211> 3300 | |
| <212> DNA | |
| <213> Homo sapiens | |
| <400> 11 | |
| tgggcaggaa ctgggcactg tgcccagggc atgcactgcc tccacgcagc aaccctcaga | 60 |
| gtcctgagct gaaccaagaa ggaggagggg gtcgggcctc cgaggaaggc ctagccgctg | 120 |
| ctgctgccag gaattccagg ttggaggggc ggcaacctcc tgccagcctt caggccactc | 180 |
| tcctgtgcct gccagaagag acagagcttg aggagagctt gaggagagca ggaaagcctc | 240 |
| ccccgttgcc cctctggatc cactgcttaa atacggacga ggacagggcc ctgtctcctc | 300 |
| agcttcaggc accaccactg acctgggaca gtgaatcgac aatgccgtct tctgtctcgt | 360 |
| ggggcatcct cctgctggca ggcctgtgct gcctggtccc tgtctccctg gctgaggatc | 420 |
| cccagggaga tgctgcccag aagacagata catcccacca tgatcaggat cacccaacct | 480 |
| tcaacaagat cacccccaac ctggctgagt tcgccttcag cctataccgc cagctggcac | 540 |
| accagtccaa cagcaccaat atcttcttct ccccagtgag catcgctaca gcctttgcaa | 600 |
| tgctctccct ggggaccaag gctgacactc acgatgaaat cctggagggc ctgaatttca | 660 |
| acctcacgga gattccggag gctcagatcc atgaaggctt ccaggaactc ctccgtaccc | 720 |
| tcaaccagcc agacagccag ctccagctga ccaccggcaa tggcctgttc ctcagcgagg | 780 |
| gcctgaagct agtggataag tttttggagg atgttaaaaa gttgtaccac tcagaagcct | 840 |
| tcactgtcaa cttcggggac accgaagagg ccaagaaaca gatcaacgat tacgtggaga | 900 |
| agggtactca agggaaaatt gtggatttgg tcaaggagct tgacagagac acagtttttg | 960 |
| ctctggtgaa ttacatcttc tttaaaggca aatgggagag accctttgaa gtcaaggaca | 1020 |
| ccgaggaaga ggacttccac gtggaccagg tgaccaccgt gaaggtgcct atgatgaagc | 1080 |
| gtttaggcat gtttaacatc cagcactgta agaagctgtc cagctgggtg ctgctgatga | 1140 |
| aatacctggg caatgccacc gccatcttct tcctgcctga tgaggggaaa ctacagcacc | 1200 |
| tggaaaatga actcacccac gatatcatca ccaagttcct ggaaaatgaa gacagaaggt | 1260 |
| ctgccagctt acatttaccc aaactgtcca ttactggaac ctatgatctg aagagcgtcc | 1320 |
| tgggtcaact gggcatcact aaggtcttca gcaatggggc tgacctctcc ggggtcacag | 1380 |
| aggaggcacc cctgaagctc tccaaggccg tgcataaggc tgtgctgacc atcgacgaga | 1440 |
| aagggactga agctgctggg gccatgtttt tagaggccat acccatgtct atcccccccg | 1500 |
| aggtcaagtt caacaaaccc tttgtcttct taatgattga acaaaatacc aagtctcccc | 1560 |
| tcttcatggg aaaagtggtg aatcccaccc aaaaataact gcctctcgct cctcaacccc | 1620 |
| tcccctccat ccctggcccc ctccctggat gacattaaag aagggttgag ctggtccctg | 1680 |
| cctgcatgtg actgtaaatc cctcccatgt tttctctgag tctccctttg cctgctgagg | 1740 |
| ctgtatgtgg gctccaggta acagtgctgt cttcgggccc cctgaactgt gttcatggag | 1800 |
| catctggctg ggtaggcaca tgctgggctt gaatccaggg gggactgaat cctcagctta | 1860 |
| cggacctggg cccatctgtt tctggagggc tccagtcttc cttgtcctgt cttggagtcc | 1920 |
| ccaagaagga atcacagggg aggaaccaga taccagccat gaccccaggc tccaccaagc | 1980 |
| atcttcatgt ccccctgctc atcccccact cccccccacc cagagttgct catcctgcca | 2040 |
| gggctggctg tgcccacccc aaggctgccc tcctgggggc cccagaactg cctgatcgtg | 2100 |
| ccgtggccca gttttgtggc atctgcagca acacaagaga gaggacaatg tcctcctctt | 2160 |
| gacccgctgt cacctaacca gactcgggcc ctgcacctct caggcacttc tggaaaatga | 2220 |
| ctgaggcaga ttcttcctga agcccattct ccatggggca acaaggacac ctattctgtc | 2280 |
| cttgtccttc catcgctgcc ccagaaagcc tcacatatct ccgtttagaa tcaggtccct | 2340 |
| tctccccaga tgaagaggag ggtctctgct ttgttttctc tatctcctcc tcagacttga | 2400 |
| ccaggcccag caggccccag aagaccatta ccctatatcc cttctcctcc ctagtcacat | 2460 |
| ggccataggc ctgctgatgg ctcaggaagg ccattgcaag gactcctcag ctatgggaga | 2520 |
| ggaagcacat cacccattga cccccgcaac ccctcccttt cctcctctga gtcccgactg | 2580 |
| gggccacatg cagcctgact tctttgtgcc tgttgctgtc cctgcagtct tcagagggcc | 2640 |
| accgcagctc cagtgccacg gcaggaggct gttcctgaat agcccctgtg gtaagggcca | 2700 |
| ggagagtcct tccatcctcc aaggccctgc taaaggacac agcagccagg aagtcccctg | 2760 |
| ggcccctagc tgaaggacag cctgctccct ccgtctctac caggaatggc cttgtcctat | 2820 |
| ggaaggcact gccccatccc aaactaatct aggaatcact gtctaaccac tcactgtcat | 2880 |
| gaatgtgtac ttaaaggatg aggttgagtc ataccaaata gtgatttcga tagttcaaaa | 2940 |
| tggtgaaatt agcaattcta catgattcag tctaatcaat ggataccgac tgtttcccac | 3000 |
| acaagtctcc tgttctctta agcttactca ctgacagcct ttcactctcc acaaatacat | 3060 |
| taaagatatg gccatcacca agccccctag gatgacacca gacctgagag tctgaagacc | 3120 |
| tggatccaag ttctgacttt tccccctgac agctgtgtga ccttcgtgaa gtcgccaaac | 3180 |
| ctctctgagc cccagtcatt gctagtaaga cctgcctttg agttggtatg atgttcaagt | 3240 |
| tagataacaa aatgtttata cccattagaa cagagaataa atagaactac atttcttgca | 3300 |
| <210> 12 | |
| <211> 1321 | |
| <212> DNA | |
| <213> Macaca mulatta | |
| <400> 12 | |
| caggacaatg ccatcttctg tctcatgggg cgtcctcctg ctggcaggcc tgtgctgcct | 60 |
| gctccccggc tctctggctg aggatcccca gggagatgct gcccagaaga cggatacatc | 120 |
| ccaccatgat caggaccacc caaccctcaa caagatcacc cccagcctgg ctgagttcgg | 180 |
| cttcagccta taccgccagc tggcacacca gtccaacagc accaatatct tcttctcccc | 240 |
| agtgagcatc gctacagcct ttgcaatgct ctccctgggg accaaggctg acactcacag | 300 |
| tgaaatcctg gagggcctga atttcaacgt cacggagatt ccggaggctc aggtccatga | 360 |
| aggcttccag gaactcctcc ataccctcaa caagccagac agccagctcc agctgaccac | 420 |
| cggcaacggc ctgttcctca acaagagcct gaaggtagtg gataagtttt tggaggatgt | 480 |
| caaaaaactg taccactcag aagccttctc tgtcaacttt gaggacaccg aagaggccaa | 540 |
| gaaacagatc aacaattacg tggagaagga aactcaaggg aaaattgtgg atttggtcaa | 600 |
| ggagcttgac agagacacag tttttgctct ggtgaattac atcttcttta aaggcaaatg | 660 |
| ggagagaccc tttgacgttg aggccaccaa ggaagaggac ttccacgtgg accaggcgac | 720 |
| caccgtgaag gtgcccatga tgaggcgttt aggcatgttt aacatctacc actgtgagaa | 780 |
| gctgtccagc tgggtgctgc tgatgaaata cctgggcaat gccaccgcca tcttcttcct | 840 |
| gcctgatgag gggaaactgc agcacctgga aaatgaactc acccatgata tcatcaccaa | 900 |
| gttcctggaa aatgaaaaca gcaggtctgc caacttacat ttacccagac tggccattac | 960 |
| tggaacctat gatctgaaga cagtcctggg ccacctgggt atcactaagg tcttcagcaa | 1020 |
| tggggctgac ctctcgggga tcacggagga ggcacccctg aagctctcca aggccgtgca | 1080 |
| taaggctgtg ctgaccatcg atgagaaagg gactgaagct gctggggcca tgtttttaga | 1140 |
| ggccataccc atgtctattc cccccgaggt caagttcaac aaaccctttg tcttcttaat | 1200 |
| gattgaacaa aataccaagt ctcccctctt catgggaaaa gtggtgaatc ccacccagaa | 1260 |
| ataactgcct gtcactcctc agcccctccc ctccatccct ggccccctcc ctgaatgaca | 1320 |
| t | 1321 |
| <210> 13 | |
| <211> 2064 | |
| <212> DNA | |
| <213> Macaca mulatta | |
| <400> 13 | |
| gcccagtctt gtgtctgcct ggcaatgggc aaggcccctt cctgcccaag ctccccgccc | 60 |
| ctccccaacc tattgcctcc gccacccgcc acccgaggcc aacttcctgg gtgggcagga | 120 |
| actgggccct gtgcccaggg cgtgcactgc ctccacgcag caaccctcag agtactgagc | 180 |
| tgagcaaagg aggaggaggg gatcagcact ctgaggaagg cctagccact gctgctgcca | 240 |
| ggaattccag ggcggcatca gtcttcagca tcaggcattt cggggtgaat tagtaaatgg | 300 |
| tagatcttgc taccagtgga acagccgcta aggattctgc agtgagagca gagggccagc | 360 |
| aaagtggtac tctcccagcg actggctgac tcacgccacc ccctccacct tggacgcagg | 420 |
| acactgtggt ttctgagcca ggtacaatga ctccttttgg tacgtgcagt ggaggctgta | 480 |
| tgctgctcag gcagagcgtc cggacagcgt gggcgggcga ctcagcgccc agcctgtgaa | 540 |
| cttagtccct gtttgctcct ccggtaactg gggtgatctt ggttaatatt caccagcagc | 600 |
| ctcccccgtt gcccctctgc acccactgct taaatacgga caaggacagg gctctgtctc | 660 |
| ctcagcctca ggcaccacca ctgacctggg acggtgaatc gacaatgcca tcttctgtct | 720 |
| catggggcgt cctcctgctg gcaggcctgt gctgcctgct ccccggctct ctggctgagg | 780 |
| atccccaggg agatgctgcc cagaagacgg atacatccca ccatgatcag gaccacccaa | 840 |
| ccctcaacaa gatcaccccc agcctggctg agttcggctt cagcctatac cgccagctgg | 900 |
| cacaccagtc caacagcacc aatatcttct tctccccagt gagcatcgct acagcctttg | 960 |
| caatgctctc cctggggacc aaggctgaca ctcacagtga aatcctggag ggcctgaatt | 1020 |
| tcaacgtcac ggagattccg gaggctcagg tccatgaagg cttccaggaa ctcctccata | 1080 |
| ccctcaacaa gccagacagc cagctccagc tgaccaccgg caacggcctg ttcctcaaca | 1140 |
| agagcctgaa ggtagtggat aagtttttgg aggatgtcaa aaaactgtac cactcagaag | 1200 |
| ccttctctgt caactttgag gacaccgaag aggccaagaa acagatcaac aattacgtgg | 1260 |
| agaaggaaac tcaagggaaa attgtggatt tggtcaagga gcttgacaga gacacagttt | 1320 |
| ttgctctggt gaattacatc ttctttaaag gcaaatggga gagacccttt gacgttgagg | 1380 |
| ccaccaagga agaggacttc cacgtggacc aggcgaccac cgtgaaggtg cccatgatga | 1440 |
| ggcgtttagg catgtttaac atctaccact gtgagaagct gtccagctgg gtgctgctga | 1500 |
| tgaaatacct gggcaatgcc accgccatct tcttcctgcc tgatgagggg aaactgcagc | 1560 |
| acctggaaaa tgaactcacc catgatatca tcaccaagtt cctggaaaat gaaaacagca | 1620 |
| ggtctgccaa cttacattta cccagactgg ccattactgg aacctatgat ctgaagacag | 1680 |
| tcctgggcca cctgggtatc actaaggtct tcagcaatgg ggctgacctc tcggggatca | 1740 |
| cggaggaggc acccctgaag ctctccaagg ccgtgcataa ggctgtgctg accatcgatg | 1800 |
| agaaagggac tgaagctgct ggggccatgt ttttagaggc catacccatg tctattcccc | 1860 |
| ccgaggtcaa gttcaacaaa ccctttgtct tcttaatgat tgaacaaaat accaagtctc | 1920 |
| ccctcttcat gggaaaagtg gtgaatccca cccagaaata actgcctgtc actcctcagc | 1980 |
| ccctcccctc catccctggc cccctccctg aatgacatta aagaagggtt gagctggtcc | 2040 |
| ctgcctgcgt gtgtgactgc aaac | 2064 |
| <210> 14 | |
| <211> 1608 | |
| <212> DNA | |
| <213> Macaca mulatta | |
| <400> 14 | |
| tcttgtgtct gcctggcaat gggcaaggcc ccttcctgcc caagctcccc gcccctcccc | 60 |
| aacctattgc ctccgccacc cgccacccga ggccaacttc ctgggtgggc aggaactggg | 120 |
| ccctgtgccc agggcgtgca ctgcctccac gcagcaaccc tcagagtact gagctgagca | 180 |
| aaggaggagg aggggatcag cactctgagg aaggcctagc cactgctgct gccaggaatt | 240 |
| ccaggacaat gccatcttct gtctcatggg gcgtcctcct gctggcaggc ctgtgctgcc | 300 |
| tgctccccgg ctctctggct gaggatcccc agggagatgc tgcccagaag acggatacat | 360 |
| cccaccatga tcaggaccac ccaaccctca acaagatcac ccccagcctg gctgagttcg | 420 |
| gcttcagcct ataccgccag ctggcacacc agtccaacag caccaatatc ttcttctccc | 480 |
| cagtgagcat cgctacagcc tttgcaatgc tctccctggg gaccaaggct gacactcaca | 540 |
| gtgaaatcct ggagggcctg aatttcaacg tcacggagat tccggaggct caggtccatg | 600 |
| aaggcttcca ggaactcctc cataccctca acaagccaga cagccagctc cagctgacca | 660 |
| ccggcaacgg cctgttcctc aacaagagcc tgaaggtagt ggataagttt ttggaggatg | 720 |
| tcaaaaaact gtaccactca gaagccttct ctgtcaactt tgaggacacc gaagaggcca | 780 |
| agaaacagat caacaattac gtggagaagg aaactcaagg gaaaattgtg gatttggtca | 840 |
| aggagcttga cagagacaca gtttttgctc tggtgaatta catcttcttt aaaggcaaat | 900 |
| gggagagacc ctttgacgtt gaggccacca aggaagagga cttccacgtg gaccaggcga | 960 |
| ccaccgtgaa ggtgcccatg atgaggcgtt taggcatgtt taacatctac cactgtgaga | 1020 |
| agctgtccag ctgggtgctg ctgatgaaat acctgggcaa tgccaccgcc atcttcttcc | 1080 |
| tgcctgatga ggggaaactg cagcacctgg aaaatgaact cacccatgat atcatcacca | 1140 |
| agttcctgga aaatgaaaac agcaggtctg ccaacttaca tttacccaga ctggccatta | 1200 |
| ctggaaccta tgatctgaag acagtcctgg gccacctggg tatcactaag gtcttcagca | 1260 |
| atggggctga cctctcgggg atcacggagg aggcacccct gaagctctcc aaggccgtgc | 1320 |
| ataaggctgt gctgaccatc gatgagaaag ggactgaagc tgctggggcc atgtttttag | 1380 |
| aggccatacc catgtctatt ccccccgagg tcaagttcaa caaacccttt gtcttcttaa | 1440 |
| tgattgaaca aaataccaag tctcccctct tcatgggaaa agtggtgaat cccacccaga | 1500 |
| aataactgcc tgtcactcct cagcccctcc cctccatccc tggccccctc cctgaatgac | 1560 |
| attaaagaag ggttgagctg gtccctgcct gcgtgtgtga ctgcaaac | 1608 |
| <210> 15 | |
| <211> 3220 | |
| <212> DNA | |
| <213> Homo sapiens | |
| <400> 15 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggctgctg gtgaatatta accaaggtca ccccagttat cggaggagca aacaggggct | 3120 |
| aagtccactg gctgggatct gagtcgcccg cctacgctgc ccggacgctt tgcctgggca | 3180 |
| gtgtacagct tccactgcac ttaccgaaag gagtcattgt | 3220 |
| <210> 16 | |
| <211> 3199 | |
| <212> DNA | |
| <213> Homo sapiens | |
| <400> 16 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcctttcct gctctcctca agctctcctc aagctctgtc | 3000 |
| tcttctggca ggcacaggag agtggcctga aggctggcag gaggttgccg cccctccaac | 3060 |
| ctggaattcc tggcagcagc agcggctagg ccttcctcgg aggcccgacc ccctcctcct | 3120 |
| tcttggttca gctcaggact ctgagggttg ctgcgtggag gcagtgcatg ccctgggcac | 3180 |
| agtgcccagt tcctgccca | 3199 |
| <210> 17 | |
| <211> 3513 | |
| <212> DNA | |
| <213> Homo sapiens | |
| <400> 17 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggctgcga aaggagtcat tgtacctggc tcagaaacca cagcgtcctg tgtccaaggt | 3120 |
| ggagggggtg gcgtgagtca gacagtctct gggagagtac cacttagctg gccctctgct | 3180 |
| ctcactgcag aatccttagt ggctgttcca ctggtagcaa gatctaccat ttactgagtc | 3240 |
| accccaaaat gcctgatgct gaagacttac tgccgccctt tcctgctctc ctcaagctct | 3300 |
| cctcaagctc tgtctcttct ggcaggcaca ggagagtggc ctgaaggctg gcaggaggtt | 3360 |
| gccgcccctc caacctggaa ttcctggcag cagcagcggc taggccttcc tcggaggccc | 3420 |
| gaccccctcc tccttcttgg ttcagctcag gactctgagg gttgctgcgt ggaggcagtg | 3480 |
| catgccctgg gcacagtgcc cagttcctgc cca | 3513 |
| <210> 18 | |
| <211> 3236 | |
| <212> DNA | |
| <213> Homo sapiens | |
| <400> 18 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcctgtgga actgagtgag cagcagcagc aatgtcccac | 3000 |
| ctttcctgct ctcctcaagc tctcctcaag ctctgtctct tctggcaggc acaggagagt | 3060 |
| ggcctgaagg ctggcaggag gttgccgccc ctccaacctg gaattcctgg cagcagcagc | 3120 |
| ggctaggcct tcctcggagg cccgaccccc tcctccttct tggttcagct caggactctg | 3180 |
| agggttgctg cgtggaggca gtgcatgccc tgggcacagt gcccagttcc tgccca | 3236 |
| <210> 19 | |
| <211> 3532 | |
| <212> DNA | |
| <213> Homo sapiens | |
| <400> 19 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggctgctg gctcagaaac cacagcgtcc tgtgtccaag gtggaggggg tggcgtgagt | 3120 |
| cagacagtct ctgggagagt accacttagc tggccctctg ctctcactgc agaatcctta | 3180 |
| gtggctgttc cactggtagc aagatctacc atttactgag tcaccccaaa atgcctgatg | 3240 |
| ctgaagactt actgccgccc tgtggaactg agtgagcagc agcagcaatg tcccaccttt | 3300 |
| cctgctctcc tcaagctctc ctcaagctct gtctcttctg gcaggcacag gagagtggcc | 3360 |
| tgaaggctgg caggaggttg ccgcccctcc aacctggaat tcctggcagc agcagcggct | 3420 |
| aggccttcct cggaggcccg accccctcct ccttcttggt tcagctcagg actctgaggg | 3480 |
| ttgctgcgtg gaggcagtgc atgccctggg cacagtgccc agttcctgcc ca | 3532 |
| <210> 20 | |
| <211> 3340 | |
| <212> DNA | |
| <213> Homo sapiens | |
| <400> 20 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggctgctg tggaactgag tgagcagcag cagcaatgtc ccacctttcc tgctctcctc | 3120 |
| aagctctcct caagctctgt ctcttctggc aggcacagga gagtggcctg aaggctggca | 3180 |
| ggaggttgcc gcccctccaa cctggaattc ctggcagcag cagcggctag gccttcctcg | 3240 |
| gaggcccgac cccctcctcc ttcttggttc agctcaggac tctgagggtt gctgcgtgga | 3300 |
| ggcagtgcat gccctgggca cagtgcccag ttcctgccca | 3340 |
| <210> 21 | |
| <211> 3495 | |
| <212> DNA | |
| <213> Homo sapiens | |
| <400> 21 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggctgctg gctcagaaac cacagcgtcc tgtgtccaag gtggaggggg tggcgtgagt | 3120 |
| cagacagtct ctgggagagt accacttagc tggccctctg ctctcactgc agaatcctta | 3180 |
| gtggctgttc cactggtagc aagatctacc atttactgag tcaccccaaa atgcctgatg | 3240 |
| ctgaagactt actgccgccc tttcctgctc tcctcaagct ctcctcaagc tctgtctctt | 3300 |
| ctggcaggca caggagagtg gcctgaaggc tggcaggagg ttgccgcccc tccaacctgg | 3360 |
| aattcctggc agcagcagcg gctaggcctt cctcggaggc ccgaccccct cctccttctt | 3420 |
| ggttcagctc aggactctga gggttgctgc gtggaggcag tgcatgccct gggcacagtg | 3480 |
| cccagttcct gccca | 3495 |
| <210> 22 | |
| <211> 3492 | |
| <212> DNA | |
| <213> Homo sapiens | |
| <400> 22 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggctggct cagaaaccac agcgtcctgt gtccaaggtg gagggggtgg cgtgagtcag | 3120 |
| acagtctctg ggagagtacc acttagctgg ccctctgctc tcactgcaga atccttagtg | 3180 |
| gctgttccac tggtagcaag atctaccatt tactgagtca ccccaaaatg cctgatgctg | 3240 |
| aagacttact gccgcccttt cctgctctcc tcaagctctc ctcaagctct gtctcttctg | 3300 |
| gcaggcacag gagagtggcc tgaaggctgg caggaggttg ccgcccctcc aacctggaat | 3360 |
| tcctggcagc agcagcggct aggccttcct cggaggcccg accccctcct ccttcttggt | 3420 |
| tcagctcagg actctgaggg ttgctgcgtg gaggcagtgc atgccctggg cacagtgccc | 3480 |
| agttcctgcc ca | 3492 |
| <210> 23 | |
| <211> 3510 | |
| <212> DNA | |
| <213> Homo sapiens | |
| <400> 23 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggcgaaag gagtcattgt acctggctca gaaaccacag cgtcctgtgt ccaaggtgga | 3120 |
| gggggtggcg tgagtcagac agtctctggg agagtaccac ttagctggcc ctctgctctc | 3180 |
| actgcagaat ccttagtggc tgttccactg gtagcaagat ctaccattta ctgagtcacc | 3240 |
| ccaaaatgcc tgatgctgaa gacttactgc cgccctttcc tgctctcctc aagctctcct | 3300 |
| caagctctgt ctcttctggc aggcacagga gagtggcctg aaggctggca ggaggttgcc | 3360 |
| gcccctccaa cctggaattc ctggcagcag cagcggctag gccttcctcg gaggcccgac | 3420 |
| cccctcctcc ttcttggttc agctcaggac tctgagggtt gctgcgtgga ggcagtgcat | 3480 |
| gccctgggca cagtgcccag ttcctgccca | 3510 |
| <210> 24 | |
| <211> 3303 | |
| <212> DNA | |
| <213> Homo sapiens | |
| <400> 24 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggctgctt tcctgctctc ctcaagctct cctcaagctc tgtctcttct ggcaggcaca | 3120 |
| ggagagtggc ctgaaggctg gcaggaggtt gccgcccctc caacctggaa ttcctggcag | 3180 |
| cagcagcggc taggccttcc tcggaggccc gaccccctcc tccttcttgg ttcagctcag | 3240 |
| gactctgagg gttgctgcgt ggaggcagtg catgccctgg gcacagtgcc cagttcctgc | 3300 |
| cca | 3303 |
| <210> 25 | |
| <211> 3300 | |
| <212> DNA | |
| <213> Homo sapiens | |
| <400> 25 | |
| tgcaagaaat gtagttctat ttattctctg ttctaatggg tataaacatt ttgttatcta | 60 |
| acttgaacat cataccaact caaaggcagg tcttactagc aatgactggg gctcagagag | 120 |
| gtttggcgac ttcacgaagg tcacacagct gtcaggggga aaagtcagaa cttggatcca | 180 |
| ggtcttcaga ctctcaggtc tggtgtcatc ctagggggct tggtgatggc catatcttta | 240 |
| atgtatttgt ggagagtgaa aggctgtcag tgagtaagct taagagaaca ggagacttgt | 300 |
| gtgggaaaca gtcggtatcc attgattaga ctgaatcatg tagaattgct aatttcacca | 360 |
| ttttgaacta tcgaaatcac tatttggtat gactcaacct catcctttaa gtacacattc | 420 |
| atgacagtga gtggttagac agtgattcct agattagttt gggatggggc agtgccttcc | 480 |
| ataggacaag gccattcctg gtagagacgg agggagcagg ctgtccttca gctaggggcc | 540 |
| caggggactt cctggctgct gtgtccttta gcagggcctt ggaggatgga aggactctcc | 600 |
| tggcccttac cacaggggct attcaggaac agcctcctgc cgtggcactg gagctgcggt | 660 |
| ggccctctga agactgcagg gacagcaaca ggcacaaaga agtcaggctg catgtggccc | 720 |
| cagtcgggac tcagaggagg aaagggaggg gttgcggggg tcaatgggtg atgtgcttcc | 780 |
| tctcccatag ctgaggagtc cttgcaatgg ccttcctgag ccatcagcag gcctatggcc | 840 |
| atgtgactag ggaggagaag ggatataggg taatggtctt ctggggcctg ctgggcctgg | 900 |
| tcaagtctga ggaggagata gagaaaacaa agcagagacc ctcctcttca tctggggaga | 960 |
| agggacctga ttctaaacgg agatatgtga ggctttctgg ggcagcgatg gaaggacaag | 1020 |
| gacagaatag gtgtccttgt tgccccatgg agaatgggct tcaggaagaa tctgcctcag | 1080 |
| tcattttcca gaagtgcctg agaggtgcag ggcccgagtc tggttaggtg acagcgggtc | 1140 |
| aagaggagga cattgtcctc tctcttgtgt tgctgcagat gccacaaaac tgggccacgg | 1200 |
| cacgatcagg cagttctggg gcccccagga gggcagcctt ggggtgggca cagccagccc | 1260 |
| tggcaggatg agcaactctg ggtggggggg agtgggggat gagcaggggg acatgaagat | 1320 |
| gcttggtgga gcctggggtc atggctggta tctggttcct cccctgtgat tccttcttgg | 1380 |
| ggactccaag acaggacaag gaagactgga gccctccaga aacagatggg cccaggtccg | 1440 |
| taagctgagg attcagtccc ccctggattc aagcccagca tgtgcctacc cagccagatg | 1500 |
| ctccatgaac acagttcagg gggcccgaag acagcactgt tacctggagc ccacatacag | 1560 |
| cctcagcagg caaagggaga ctcagagaaa acatgggagg gatttacagt cacatgcagg | 1620 |
| cagggaccag ctcaaccctt ctttaatgtc atccagggag ggggccaggg atggagggga | 1680 |
| ggggttgagg agcgagaggc agttattttt gggtgggatt caccactttt cccatgaaga | 1740 |
| ggggagactt ggtattttgt tcaatcatta agaagacaaa gggtttgttg aacttgacct | 1800 |
| cgggggggat agacatgggt atggcctcta aaaacatggc cccagcagct tcagtccctt | 1860 |
| tctcgtcgat ggtcagcaca gccttatgca cggccttgga gagcttcagg ggtgcctcct | 1920 |
| ctgtgacccc ggagaggtca gccccattgc tgaagacctt agtgatgccc agttgaccca | 1980 |
| ggacgctctt cagatcatag gttccagtaa tggacagttt gggtaaatgt aagctggcag | 2040 |
| accttctgtc ttcattttcc aggaacttgg tgatgatatc gtgggtgagt tcattttcca | 2100 |
| ggtgctgtag tttcccctca tcaggcagga agaagatggc ggtggcattg cccaggtatt | 2160 |
| tcatcagcag cacccagctg gacagcttct tacagtgctg gatgttaaac atgcctaaac | 2220 |
| gcttcatcat aggcaccttc acggtggtca cctggtccac gtggaagtcc tcttcctcgg | 2280 |
| tgtccttgac ttcaaagggt ctctcccatt tgcctttaaa gaagatgtaa ttcaccagag | 2340 |
| caaaaactgt gtctctgtca agctccttga ccaaatccac aattttccct tgagtaccct | 2400 |
| tctccacgta atcgttgatc tgtttcttgg cctcttcggt gtccccgaag ttgacagtga | 2460 |
| aggcttctga gtggtacaac tttttaacat cctccaaaaa cttatccact agcttcaggc | 2520 |
| cctcgctgag gaacaggcca ttgccggtgg tcagctggag ctggctgtct ggctggttga | 2580 |
| gggtacggag gagttcctgg aagccttcat ggatctgagc ctccggaatc tccgtgaggt | 2640 |
| tgaaattcag gccctccagg atttcatcgt gagtgtcagc cttggtcccc agggagagca | 2700 |
| ttgcaaaggc tgtagcgatg ctcactgggg agaagaagat attggtgctg ttggactggt | 2760 |
| gtgccagctg gcggtatagg ctgaaggcga actcagccag gttgggggtg atcttgttga | 2820 |
| aggttgggtg atcctgatca tggtgggatg tatctgtctt ctgggcagca tctccctggg | 2880 |
| gatcctcagc cagggagaca gggaccaggc agcacaggcc tgccagcagg aggatgcccc | 2940 |
| acgagacaga agacggcatt gtcgattcac tgtcccaggt cagtggtggt gcctgaagct | 3000 |
| gaggagacag ggccctgtcc tcgtccgtat ttaagcagtg gatccagagg ggcaacgggg | 3060 |
| gaggctttcc tgctctcctc aagctctcct caagctctgt ctcttctggc aggcacagga | 3120 |
| gagtggcctg aaggctggca ggaggttgcc gcccctccaa cctggaattc ctggcagcag | 3180 |
| cagcggctag gccttcctcg gaggcccgac cccctcctcc ttcttggttc agctcaggac | 3240 |
| tctgagggtt gctgcgtgga ggcagtgcat gccctgggca cagtgcccag ttcctgccca | 3300 |
| <210> 26 | |
| <211> 1321 | |
| <212> DNA | |
| <213> Macaca mulatta | |
| <400> 26 | |
| atgtcattca gggagggggc cagggatgga ggggaggggc tgaggagtga caggcagtta | 60 |
| tttctgggtg ggattcacca cttttcccat gaagagggga gacttggtat tttgttcaat | 120 |
| cattaagaag acaaagggtt tgttgaactt gacctcgggg ggaatagaca tgggtatggc | 180 |
| ctctaaaaac atggccccag cagcttcagt ccctttctca tcgatggtca gcacagcctt | 240 |
| atgcacggcc ttggagagct tcaggggtgc ctcctccgtg atccccgaga ggtcagcccc | 300 |
| attgctgaag accttagtga tacccaggtg gcccaggact gtcttcagat cataggttcc | 360 |
| agtaatggcc agtctgggta aatgtaagtt ggcagacctg ctgttttcat tttccaggaa | 420 |
| cttggtgatg atatcatggg tgagttcatt ttccaggtgc tgcagtttcc cctcatcagg | 480 |
| caggaagaag atggcggtgg cattgcccag gtatttcatc agcagcaccc agctggacag | 540 |
| cttctcacag tggtagatgt taaacatgcc taaacgcctc atcatgggca ccttcacggt | 600 |
| ggtcgcctgg tccacgtgga agtcctcttc cttggtggcc tcaacgtcaa agggtctctc | 660 |
| ccatttgcct ttaaagaaga tgtaattcac cagagcaaaa actgtgtctc tgtcaagctc | 720 |
| cttgaccaaa tccacaattt tcccttgagt ttccttctcc acgtaattgt tgatctgttt | 780 |
| cttggcctct tcggtgtcct caaagttgac agagaaggct tctgagtggt acagtttttt | 840 |
| gacatcctcc aaaaacttat ccactacctt caggctcttg ttgaggaaca ggccgttgcc | 900 |
| ggtggtcagc tggagctggc tgtctggctt gttgagggta tggaggagtt cctggaagcc | 960 |
| ttcatggacc tgagcctccg gaatctccgt gacgttgaaa ttcaggccct ccaggatttc | 1020 |
| actgtgagtg tcagccttgg tccccaggga gagcattgca aaggctgtag cgatgctcac | 1080 |
| tggggagaag aagatattgg tgctgttgga ctggtgtgcc agctggcggt ataggctgaa | 1140 |
| gccgaactca gccaggctgg gggtgatctt gttgagggtt gggtggtcct gatcatggtg | 1200 |
| ggatgtatcc gtcttctggg cagcatctcc ctggggatcc tcagccagag agccggggag | 1260 |
| caggcagcac aggcctgcca gcaggaggac gccccatgag acagaagatg gcattgtcct | 1320 |
| g | 1321 |
| <210> 27 | |
| <211> 2064 | |
| <212> DNA | |
| <213> Macaca mulatta | |
| <400> 27 | |
| gtttgcagtc acacacgcag gcagggacca gctcaaccct tctttaatgt cattcaggga | 60 |
| gggggccagg gatggagggg aggggctgag gagtgacagg cagttatttc tgggtgggat | 120 |
| tcaccacttt tcccatgaag aggggagact tggtattttg ttcaatcatt aagaagacaa | 180 |
| agggtttgtt gaacttgacc tcggggggaa tagacatggg tatggcctct aaaaacatgg | 240 |
| ccccagcagc ttcagtccct ttctcatcga tggtcagcac agccttatgc acggccttgg | 300 |
| agagcttcag gggtgcctcc tccgtgatcc ccgagaggtc agccccattg ctgaagacct | 360 |
| tagtgatacc caggtggccc aggactgtct tcagatcata ggttccagta atggccagtc | 420 |
| tgggtaaatg taagttggca gacctgctgt tttcattttc caggaacttg gtgatgatat | 480 |
| catgggtgag ttcattttcc aggtgctgca gtttcccctc atcaggcagg aagaagatgg | 540 |
| cggtggcatt gcccaggtat ttcatcagca gcacccagct ggacagcttc tcacagtggt | 600 |
| agatgttaaa catgcctaaa cgcctcatca tgggcacctt cacggtggtc gcctggtcca | 660 |
| cgtggaagtc ctcttccttg gtggcctcaa cgtcaaaggg tctctcccat ttgcctttaa | 720 |
| agaagatgta attcaccaga gcaaaaactg tgtctctgtc aagctccttg accaaatcca | 780 |
| caattttccc ttgagtttcc ttctccacgt aattgttgat ctgtttcttg gcctcttcgg | 840 |
| tgtcctcaaa gttgacagag aaggcttctg agtggtacag ttttttgaca tcctccaaaa | 900 |
| acttatccac taccttcagg ctcttgttga ggaacaggcc gttgccggtg gtcagctgga | 960 |
| gctggctgtc tggcttgttg agggtatgga ggagttcctg gaagccttca tggacctgag | 1020 |
| cctccggaat ctccgtgacg ttgaaattca ggccctccag gatttcactg tgagtgtcag | 1080 |
| ccttggtccc cagggagagc attgcaaagg ctgtagcgat gctcactggg gagaagaaga | 1140 |
| tattggtgct gttggactgg tgtgccagct ggcggtatag gctgaagccg aactcagcca | 1200 |
| ggctgggggt gatcttgttg agggttgggt ggtcctgatc atggtgggat gtatccgtct | 1260 |
| tctgggcagc atctccctgg ggatcctcag ccagagagcc ggggagcagg cagcacaggc | 1320 |
| ctgccagcag gaggacgccc catgagacag aagatggcat tgtcgattca ccgtcccagg | 1380 |
| tcagtggtgg tgcctgaggc tgaggagaca gagccctgtc cttgtccgta tttaagcagt | 1440 |
| gggtgcagag gggcaacggg ggaggctgct ggtgaatatt aaccaagatc accccagtta | 1500 |
| ccggaggagc aaacagggac taagttcaca ggctgggcgc tgagtcgccc gcccacgctg | 1560 |
| tccggacgct ctgcctgagc agcatacagc ctccactgca cgtaccaaaa ggagtcattg | 1620 |
| tacctggctc agaaaccaca gtgtcctgcg tccaaggtgg agggggtggc gtgagtcagc | 1680 |
| cagtcgctgg gagagtacca ctttgctggc cctctgctct cactgcagaa tccttagcgg | 1740 |
| ctgttccact ggtagcaaga tctaccattt actaattcac cccgaaatgc ctgatgctga | 1800 |
| agactgatgc cgccctggaa ttcctggcag cagcagtggc taggccttcc tcagagtgct | 1860 |
| gatcccctcc tcctcctttg ctcagctcag tactctgagg gttgctgcgt ggaggcagtg | 1920 |
| cacgccctgg gcacagggcc cagttcctgc ccacccagga agttggcctc gggtggcggg | 1980 |
| tggcggaggc aataggttgg ggaggggcgg ggagcttggg caggaagggg ccttgcccat | 2040 |
| tgccaggcag acacaagact gggc | 2064 |
| <210> 28 | |
| <211> 1608 | |
| <212> DNA | |
| <400> 28 | |
| <213> Macaca mulatta | |
| <400> 28 | |
| gtttgcagtc acacacgcag gcagggacca gctcaaccct tctttaatgt cattcaggga | 60 |
| gggggccagg gatggagggg aggggctgag gagtgacagg cagttatttc tgggtgggat | 120 |
| tcaccacttt tcccatgaag aggggagact tggtattttg ttcaatcatt aagaagacaa | 180 |
| agggtttgtt gaacttgacc tcggggggaa tagacatggg tatggcctct aaaaacatgg | 240 |
| ccccagcagc ttcagtccct ttctcatcga tggtcagcac agccttatgc acggccttgg | 300 |
| agagcttcag gggtgcctcc tccgtgatcc ccgagaggtc agccccattg ctgaagacct | 360 |
| tagtgatacc caggtggccc aggactgtct tcagatcata ggttccagta atggccagtc | 420 |
| tgggtaaatg taagttggca gacctgctgt tttcattttc caggaacttg gtgatgatat | 480 |
| catgggtgag ttcattttcc aggtgctgca gtttcccctc atcaggcagg aagaagatgg | 540 |
| cggtggcatt gcccaggtat ttcatcagca gcacccagct ggacagcttc tcacagtggt | 600 |
| agatgttaaa catgcctaaa cgcctcatca tgggcacctt cacggtggtc gcctggtcca | 660 |
| cgtggaagtc ctcttccttg gtggcctcaa cgtcaaaggg tctctcccat ttgcctttaa | 720 |
| agaagatgta attcaccaga gcaaaaactg tgtctctgtc aagctccttg accaaatcca | 780 |
| caattttccc ttgagtttcc ttctccacgt aattgttgat ctgtttcttg gcctcttcgg | 840 |
| tgtcctcaaa gttgacagag aaggcttctg agtggtacag ttttttgaca tcctccaaaa | 900 |
| acttatccac taccttcagg ctcttgttga ggaacaggcc gttgccggtg gtcagctgga | 960 |
| gctggctgtc tggcttgttg agggtatgga ggagttcctg gaagccttca tggacctgag | 1020 |
| cctccggaat ctccgtgacg ttgaaattca ggccctccag gatttcactg tgagtgtcag | 1080 |
| ccttggtccc cagggagagc attgcaaagg ctgtagcgat gctcactggg gagaagaaga | 1140 |
| tattggtgct gttggactgg tgtgccagct ggcggtatag gctgaagccg aactcagcca | 1200 |
| ggctgggggt gatcttgttg agggttgggt ggtcctgatc atggtgggat gtatccgtct | 1260 |
| tctgggcagc atctccctgg ggatcctcag ccagagagcc ggggagcagg cagcacaggc | 1320 |
| ctgccagcag gaggacgccc catgagacag aagatggcat tgtcctggaa ttcctggcag | 1380 |
| cagcagtggc taggccttcc tcagagtgct gatcccctcc tcctcctttg ctcagctcag | 1440 |
| tactctgagg gttgctgcgt ggaggcagtg cacgccctgg gcacagggcc cagttcctgc | 1500 |
| ccacccagga agttggcctc gggtggcggg tggcggaggc aataggttgg ggaggggcgg | 1560 |
| ggagcttggg caggaagggg ccttgcccat tgccaggcag acacaaga | 1608 |

1. A double stranded RNA (dsRNA) agent that inhibits expression of a serine peptidase inhibitor, clade A, member 1 (Serpina1) gene, comprising a sense strand and an antisense strand forming a double stranded region, said antisense strand comprising a region of complementarity to an mRNA encoding Serpina1, wherein the region of complementarity comprises at least 15 contiguous nucleotides differing by no more than 3 nucleotides from the nucleotide sequence of SEQ ID NO:15,
wherein said antisense strand comprises at least one thermally destabilizing modification within the first 9 nucleotide positions of the 5′ region or a precursor thereof,
wherein said sense strand comprises an ASGPR ligand,
wherein each of the sense strand and the antisense strand are independently 14 to 40 nucleotides in length.

2. The dsRNA agent according to claim 1, wherein the dsRNA agent comprises at least four nucleotides comprising a 2′-fluoro modification; or
wherein the dsRNA agent has the following characteristics:
a) the thermally destabilizing modification is located in position 4-8 of the 5′ region of the antisense strand;
b) each of the sense and antisense strands independently comprise at least two nucleotides comprising a 2′-fluoro modification; and
c) an ASGPR ligand attached to either end of the sense strand.

3. The dsRNA agent according to claim 1, wherein the antisense strand has at least two of the following characteristics:
a) the thermally destabilizing modification is located in position 4 to 8 of the antisense strand;
b) at least two nucleotides comprise a 2′-fluoro modification;
c) a phosphorothioate internucleotide linkage between nucleotide positions 1 and 2 (counting from the 5′ end);
d) a length of 18 to 35 nucleotides.

4. The dsRNA agent according to claim 1, having at least one of the following characteristics:
a) the ASGPR ligand attached to either end of the sense strand;
b) at least two nucleotides comprise a 2′-fluoro modification;
c) the double stranded region spans at least 19 nucleotide positions and wherein the thermally destabilizing modification is located within said double stranded region.

5. The dsRNA agent according to claim 1, wherein the thermally destabilizing modification is selected from the group consisting of 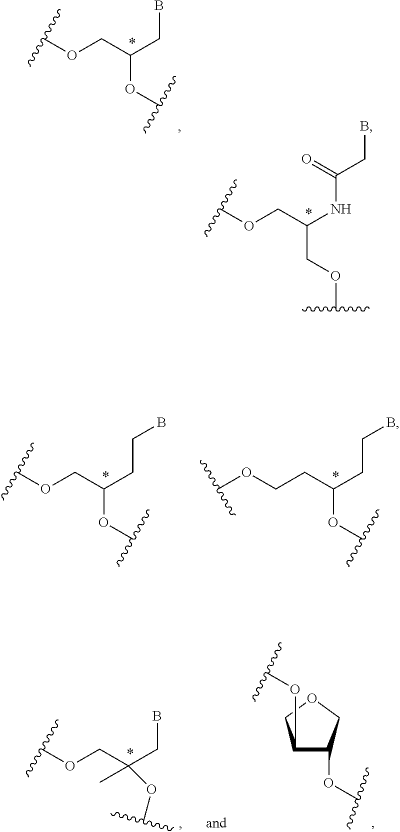
wherein B is nucleobase.

6. The dsRNA agent according to claim 1, wherein the ASGPR ligand is one or more GalNAc derivatives attached through a bivalent or trivalent branched linker.

7. A double stranded RNA (dsRNA) agent that inhibits expression of a serine peptidase inhibitor, clade A, member 1 (Serpina1) target gene sequence, comprising a sense strand and an antisense strand, said antisense strand comprising a region of complementarity to an mRNA encoding Serpina1, wherein the region of complementarity comprises at least 15 contiguous nucleotides differing by no more than 3 nucleotides from the nucleotide sequence of SEQ ID NO:15,
wherein the antisense strand comprises at least one thermally destabilizing modification within the first 9 nucleotide positions of the 5′ region,
wherein each of the sense strand and the antisense strand are independently 14 to 40 nucleotides in length, and
wherein the dsRNA agent has a melting temperature of from about 40° C. to about 80° C.

8. The dsRNA agent of claim 7, further comprising an ASGPR ligand.

9. The dsRNA agent of claim 8, wherein the ASGPR ligand is one or more GalNAc derivatives attached through a bivalent or trivalent branched linker.

10. The dsRNA agent of claim 1, wherein each strand has 15-30 nucleotides; or 19-30 nucleotides.

11. The dsRNA agent of claim 1, wherein the antisense strand comprises a region of complementarity comprising at least 15 contiguous nucleotides differing by no more than 3 nucleotides from nucleotides 1440-1480 of SEQ ID NO:1.

12. The dsRNA agent of claim 1, wherein the region of complementarity comprises at least 15 contiguous nucleotides differing by no more than 3 nucleotides from the nucleotide sequence 5′-UUUUGUUCAAUCAUUAAGAAGAC-3′ (SEQ ID NO: 419).

13. An isolated cell containing the dsRNA agent of claim 1.

14. A pharmaceutical composition comprising the dsRNA agent of claim 1.

15. A method of inhibiting Serpina1 expression in a cell, the method comprising contacting the cell with the dsRNA agent of claim 1, thereby inhibiting expression of the Serpina1 gene in the cell.

16. The method of claim 15, wherein said cell is within a subject.

17. The method of claim 16, wherein the subject is a human.

18. A method of treating a subject having a Serpina1 associated disease, comprising administering to the subject a therapeutically effective amount of the dsRNA agent of claim 1, thereby treating said subject.

19. A method of inhibiting development of hepatocellular carcinoma in a subject having a Serpina1 deficiency variant, comprising administering to the subject a therapeutically effective amount of the dsRNA agent of claim 1, thereby inhibiting development of hepatocellular carcinoma in the subject.

20. A method of reducing the accumulation of misfolded Serpina1 in the liver of a subject having a Serpina1 deficiency variant, comprising administering to the subject a therapeutically effective amount of the dsRNA agent of claim 1, thereby reducing the accumulation of misfolded Serpina1 in the liver of the subject.
